SEQUENCE LISTING (29 sequences)

SEQ ID NO: 1 - DNA, 1140 bp, Bacillus sp-62449
  CDS (1)..(1137)
  misc_feature (1)..(3): The first amino acid (position -28) in the polypeptide shown in SEQ ID NO 2 and encoded by the polynucleotide shown in SEQ ID NO1 should be Met, not Val. When the first codon is gtg a Met is inserted though gtg normally codes for V
  sig_peptide (1)..(84)
  mat_peptide (85)..(1137)
gtggttaaaattaaaattaacaatagtattagaattgtaatgctgacgctaataatgatgtccgtttcagtggtggcttatgcgtacaacccagtaacagaggacgaactatatcattcgttcgatagtcatgatgctcggaactggcagatttctgatggttggagaaatggcgatgattttttcggttgccattggagtcaaaacagggttaattttaatcgtggtgaaatggaactatctcttcgtacaaattattcatactcagctccgtataattatgagtgtgcagagtatgcgacgagtaatttctatggatatggtttgtacgaagtatctatgaaaccagccaatgtatcaggagtgatttcttctttcttcacgtatacaggtccttcatataatggagcaccttgggatgagattgatattgaatttctaggaaacgacacgacaaaagttcaattcaattattacacgaacggtgtaggaggaaatgaaataatttacgatttaggatttgatgctgcaaatagttttaatacgtatgcgtttgattggcaagagaattatattagctggtatgttaatgggaacttggtagctacagcaacagaaaatattccaagtaacccgagtaaaatcatgatgaatgtgtggaatacgtacggaattgatgaatgggcaggggcatatggaggagaagccgctaatgccacctatgaatgggtacggtatacaccgaataatggaaacacaactccttccactgctcctgactttcaattgcaagcgtgtgattactcagattcaagtgggatcacatcttggtcttgtggggtagggacctttcattctagtaattggattaaatttgatagcgttgatttatctacagggtataatgcatttgctgtcagctatacttctccgggaagtggtagttttgatattagactaggtagtcctcatggtcaaagaattggtactgtaaactatggtgcaactggtggttggtctaactacgagtggagtggtaccccgtcattagatgtgaccgtaagaggagcacatgatatatacattgtagctacgagcggagcggctaatctaagggaattttggtttaaaaatgaataa

SEQ ID NO: 2 - protein, 379 aa, Bacillus sp-62449
VVKIKINNSIRIVMLTLIMMSVSVVAYAYNPVTEDELYHSFDSHDARNWQISDGWRNGDDFFGCHWSQNRVNFNRGEMELSLRTNYSYSAPYNYECAEYATSNFYGYGLYEVSMKPANVSGVISSFFTYTGPSYNGAPWDEIDIEFLGNDTTKVQFNYYTNGVGGNEIIYDLGFDAANSFNTYAFDWQENYISWYVNGNLVATATENIPSNPSKIMMNVWNTYGIDEWAGAYGGEAANATYEWVRYTPNNGNTTPSTAPDFQLQACDYSDSSGITSWSCGVGTFHSSNWIKFDSVDLSTGYNAFAVSYTSPGSGSFDIRLGSPHGQRIGTVNYGATGGWSNYEWSGTPSLDVTVRGAHDIYIVATSGAANLREFWFKNE

SEQ ID NO: 3 - protein, 379 aa, Bacillus sp-62449
  SIGNAL (1)..(28)
  mat_peptide (29)..(379)
MVKIKINNSIRIVMLTLIMMSVSVVAYAYNPVTEDELYHSFDSHDARNWQISDGWRNGDDFFGCHWSQNRVNFNRGEMELSLRTNYSYSAPYNYECAEYATSNFYGYGLYEVSMKPANVSGVISSFFTYTGPSYNGAPWDEIDIEFLGNDTTKVQFNYYTNGVGGNEIIYDLGFDAANSFNTYAFDWQENYISWYVNGNLVATATENIPSNPSKIMMNVWNTYGIDEWAGAYGGEAANATYEWVRYTPNNGNTTPSTAPDFQLQACDYSDSSGITSWSCGVGTFHSSNWIKFDSVDLSTGYNAFAVSYTSPGSGSFDIRLGSPHGQRIGTVNYGATGGWSNYEWSGTPSLDVTVRGAHDIYIVATSGAANLREFWFKNE

SEQ ID NO: 4 - DNA, 831 bp, Bacillus akibai
  CDS (1)..(828)
  sig_peptide (1)..(93)
  mat_peptide (94)..(828)
atgaaaaagaaatttgttttattttctatgtgtttattattgtttagcggcctcataactggattagttcaaagtccacaagttgctgaagcagcagaaagaccaattgggactacatttgttgaaacatttgaatcatatgactcagaacgttggtcgaaagcgggagtttggacaaatggacaaatgtttaatgcaacatggtatccagaacaagttactttttctgatggtaagatgaagttgcaaattgataaagaagacaatgaaactgcgagcccgccatacaaagctggagaacttcgtacaaacgatttttatcactacgggttgtttgaagtgagtatgaaacctgcaaaatcaacgggaacagtctcttcatttttcacctatactggaccttgggattgggataatgatccatgggatgaaattgatatcgaatttttaggtaaggatactactaaaatacaatttaattattttacaaacggagtaggcggaaatgagcattaccatgaattaggatttgatgcagcagatgattttaatacgtatgcttttgagtggagaccagaatctattcgttggtttgtaaatggtgaactggttcatacagcaacagaaaatataccacaaacaccacaaaaaataatgatgaacttatggcctggtattggagtagacgggtggactggtagatttaatggagaagatactcctgtagttacacagtacgattgggtgaagtatacaccacttgaggaactgggctgttacaatgagaaaaataataaatacaagaaatgtaagaaaacgaaggtaaaatag

SEQ ID NO: 5 - protein, 276 aa, Bacillus akibai
MKKKFVLFSMCLLLFSGLITGLVQSPQVAEAAERPIGTTFVETFESYDSERWSKAGVWTNGQMFNATWYPEQVTFSDGKMKLQIDKEDNETASPPYKAGELRTNDFYHYGLFEVSMKPAKSTGTVSSFFTYTGPWDWDNDPWDEIDIEFLGKDTTKIQFNYFTNGVGGNEHYHELGFDAADDFNTYAFEWRPESIRWFVNGELVHTATENIPQTPQKIMMNLWPGIGVDGWTGRFNGEDTPVVTQYDWVKYTPLEELGCYNEKNNKYKKCKKTKVK

SEQ ID NO: 6 - DNA, 714 bp, Bacillus agaradhaerens
  CDS (1)..(711)
  sig_peptide (1)..(45)
  mat_peptide (46)..(711)
atgttgacgttactaatgatgtctttcgcgggtgcggcatatgcacataatccagtaacagatgaagaagtctatcattcgtttaacagtcatgattggcaaaactggaatatgtctgacggttggaaaaatgatgattactttttcgggtgtcattggagtcagaacagagttaacttttatggtgggcaaatggagttgtcactgcgtacaaactattcatacgcacctccttacaactatgagtgtgcggagtatacgaccaataatttttatggatatggattatacgaggtatctatgaaaccagcaaaggtatcaggggtcatttcttctttcttcacgtatacagggccttcctataatggagccccttgggatgaaattgacattgaatttttaggaaacgacacgactaaggttcaattcaattattacacagatggcgtaggagggaatgaaatactttatgacttaggattcgatgcagcggatagttataatacgtatgcattcgattggcaagaaaattatattaattggtatgttaatggccaacttgttgcaacagcaacggaaaacatacctagtaatcctagtaaaattatgatgaacatttggaatacgtatggtattgacgagtgggcaggaaggtattatggagaggatgccaatgcttcatataattgggttcgctatacacctaaccgttaa

SEQ ID NO: 7 - protein, 237 aa, Bacillus agaradhaerens
MLTLLMMSFAGAAYAHNPVTDEEVYHSFNSHDWQNWNMSDGWKNDDYFFGCHWSQNRVNFYGGQMELSLRTNYSYAPPYNYECAEYTTNNFYGYGLYEVSMKPAKVSGVISSFFTYTGPSYNGAPWDEIDIEFLGNDTTKVQFNYYTDGVGGNEILYDLGFDAADSYNTYAFDWQENYINWYVNGQLVATATENIPSNPSKIMMNIWNTYGIDEWAGRYYGEDANASYNWVRYTPNR

SEQ ID NO: 8 - DNA, 732 bp, Bacillus mojavensis
  CDS (1)..(729)
  sig_peptide (1)..(87)
  mat_peptide (88)..(729)
atgtcttatcgtatgaaacgagtattgttgcttcttgttactgggttgtttatgagtttgtctgcattcacttctactgcctcggctcaaacaggtggatcgttttttgacccctttaatggctacaactccggtttttggcaaaaggcaaatggctattcgaatggaaatatgtttaactgtacctggcgtgcaaataacgtatcaatgacgtcattaggggaaatgcgtttggcgctaacaagtccatcttataacaagtttgactgcggggaaaaccgctctgttcaaacatatggctatggactttatgaagtcaggatgaaaccagctaaaaacgtaggaattgtttcatcgttcttcacttacacaggtccaacagatggaactccttgggatgagattgatatcgaatttttaggaaaagacacaacaaaggttcaatttaactattatacaaatggtgtaggaaaccatgagaagctcgtggatctcggatttgatgctgccaacgcctatcatacgtatgcgttcgattggcagccaaactctattaaatggtatgtcgatgggcaattaaaacatactgcgacaagccaaattccgacaacaccaggtaagatcatgatgaacttgtggaatggtacgggtgtagatgaatggctcggttcctacaatggtgtgacaccgctatacgctcattacgactgggtgcgctatacaaaaaaataa

SEQ ID NO: 9 - protein, 243 aa, Bacillus mojavensis
MSYRMKRVLLLLVTGLFMSLSAFTSTASAQTGGSFFDPFNGYNSGFWQKANGYSNGNMFNCTWRANNVSMTSLGEMRLALTSPSYNKFDCGENRSVQTYGYGLYEVRMKPAKNVGIVSSFFTYTGPTDGTPWDEIDIEFLGKDTTKVQFNYYTNGVGNHEKLVDLGFDAANAYHTYAFDWQPNSIKWYVDGQLKHTATSQIPTTPGKIMMNLWNGTGVDEWLGSYNGVTPLYAHYDWVRYTKK

SEQ ID NO: 10 - protein, 27 aa, Bacillus clausii
  SIGNAL (1)..(27): Secretion signal
MKKPLGKIVASTALLISVAFSSSIASA

SEQ ID NO: 11 - protein, 8 aa, Artificial Sequence
  N-terminal poly histidine affinity purification tag
HHHHHHPR

SEQ ID NO: 12 - protein, 483 aa, Bacillus sp.
HHNGTNGTMMQYFEWYLPNDGNHWNRLRSDASNLKDKGISAVWIPPAWKGASQNDVGYGAYDLYDLGEFNQKGTIRTKYGTRNQLQAAVNALKSNGIQVYGDVVMNHKGGADATEMVKAVEVNPNNRNQEVSGEYTIEAWTKFDFPGRGNTHSNFKWRWYHFDGVDWDQSRKLNNRIYKFRGKGWDWEVDTEFGNYDYLMYADIDMDHPEVVNELRNWGVWYTNTLGLDGFRIDAVKHIKYSFTRDWINHVRSATGKNMFAVAEFWKNDLGAIENYLNKTNWNHSVFDVPLHYNLYNASKSGGNYDMRQIFNGTVVQKHPMHAVTFVDNHDSQPEEALESFVEEWFKPLAYALTLTREQGYPSVFYGDYYGIPTHGVPAMKSKIDPILEARQKYAYGRQNDYLDHHNIIGWTREGNTAHPNSGLATIMSDGAGGNKWMFVGRNKAGQVWTDITGNKAGTVTINADGWGNFSVNGGSVSIWVNK

SEQ ID NO: 13 - protein, 483 aa, Bacillus licheniformis [sequence omitted]

SEQ ID NO: 14 - protein, 515 aa, Bacillus stearothermophilus
  MISC_FEATURE: corresponding to SEQ ID NO 6 in WO 2002/010355
AAPFNGTMMQYFEWYLPDDGTLWTKVANEANNLSSLGITALWLPPAYKGTSRSDVGYGVYDLYDLGEFNQKGTVRTKYGTKAQYLQAIQAAHAAGMQVYADVVFDHKGGADGTEWVDAVEVNPSDRNQEISGTYQIQAWTKFDFPGRGNTYSSFKWRWYHFDGVDWDESRKLSRIYKFRGIGKAWDWEVDTENGNYDYLMYADLDMDHPEVVTELKNWGKWYVNTTNIDGFRLDAVKHIKFSFFPDWLSYVRSQTGKPLFTVGEYWSYDINKLHNYITKTDGTMSLFDAPLHNKFYTASKSGGAFDMRTLMTNTLMKDQPTLAVTFVDNHDTEPGQALQSWVDPWFKPLAYAFILTRQEGYPCVFYGDYYGIPQYNIPSLKSKIDPLLIARRDYAYGTQHDYLDHSDIIGWTREGGTEKPGSGLAALITDGPGGSKWMYVGKQHAGKVFYDLTGNRSDTVTINSDGWGEFKVNGGSVSVWVPRKTTVSTIARPITTRPWTGEFVRWTEPRLVAWP

SEQ ID NO: 15 - protein, 481 aa, Artificial Sequence
  Residues 1-33 of SEQ ID NO 6 of WO 2006/066594 and residues 36-483 of SEQ ID NO 4 of WO 2006/066594
  MISC_FEATURE: corresponding to the hybrid polypeptide comprising residues 1-33 of SEQ ID NO 6 of WO 2006/066594 and residues 36-483 of SEQ ID NO 4 of WO 2006/066594
VNGTLMQYFEWYTPNDGQHWKRLQNDAEHLSDIGITAVWIPPAYKGTSQADVGYGAYDLYDLGEFHQKGTVRTKYGTKGELQSAIKSLHSRDINVYGDVVINHKGGADATEDVTAVEVDPADRNRVISGEHLIKAWTHFHFPGRGSTYSDFKWHWYHFDGTDWDESRKLNRIYKFQGKAWDWEVSNENGNYDYLMYADIDYDHPDVAAEIKRWGTWYANELQLDGFRLDAVKHIKFSFLRDWVNHVREKTGKEMFTVAEYWQNDLGALENYLNKTNFNHSVFDVPLHYQFHAASTQGGGYDMRKLLNGTVVSKHPLKSVTFVDNHDTQPGQSLESTVQTWFKPLAYAFILTRESGYPQVFYGDMYGTKGDSQREIPALKHKIEPILKARKQYAYGAQHDYFDHHDIVGWTREGDSSVANSGLAALITDGPGGAKRMYVGRQNAGETWHDITGNRSEPVVINSEGWGEFHVNGGSVSIYVQR

SEQ ID NO: 16 - protein, 485 aa, Bacillus sp.
  MISC_FEATURE: corresponding to SEQ ID NO 6 of WO 2002/019467
HHNGTNGTMMQYFEWYLPNDGNHWNRLNSDASNLKSKGITAVWIPPAWKGASQNDVGYGAYDLYDLGEFNQKGTVRTKYGTRSQLQAAVTSLKNNGIQVYGDVVMNHKGGADATEMVRAVEVNPNNRNQEVTGEYTIEAWTRFDFPGRGNTHSSFKWRWYHFDGVDWDQSRRLNNRIYKFRGHGKAWDWEVDTENGNYDYLMYADIDMDHPEVVNELRNWGVWYTNTLGLDGFRIDAVKHIKYSFTRDWINHVRSATGKNMFAVAEFWKNDLGAIENYLQKTNWNHSVFDVPLHYNLYNASKSGGNYDMRNIFNGTVVQRHPSHAVTFVDNHDSQPEEALESFVEEWFKPLAYALTLTREQGYPSVFYGDYYGIPTHGVPAMRSKIDPILEARQKYAYGKQNDYLDHHNIIGWTREGNTAHPNSGLATIMSDGAGGSKWMFVGRNKAGQVWSDITGNRTGTVTINADGWGNFSVNGGSVSIWVNK

SEQ ID NO: 17 - protein, 485 aa, Bacillus sp. NCIB 12512
  MISC_FEATURE: corresponding to SEQ ID NO 1 of WO 1996/023873
HHNGTNGTMMQYFEWYLPNDGNHWNRLRDDAANLKSKGITAVWIPPAWKGTSQNDVGYGAYDLYDLGEFNQKGTVRTKYGTRNQLQAAVTSLKNNGIQVYGDVVMNHKGGADGTEIVNAVEVNRSNRNQETSGEYAIEAWTKFDFPGRGNNHSSFKWRWYHFDGTDWDQSRQLQNKIYKFRGTGKAWDWEVDTENGNYDYLMYADVDMDHPEVIHELRNWGVWYTNTLNLDGFRIDAVKHIKYSFTRDWLTHVRNTTGKPMFAVAEFWKNDLGAIENYLNKTSWNHSVFDVPLHYNLYNASNSGGYYDMRNILNGSVVQKHPTHAVTFVDNHDSQPGEALESFVQQWFKPLAYALVLTREQGYPSVFYGDYYGIPTHGVPAMKSKIDPLLQARQTFAYGTQHDYFDHHDIIGWTREGNSSHPNSGLATIMSDGPGGNKWMYVGKNKAGQVWRDITGNRTGTVTINADGWGNFSVNGGSVSVWVKQ

SEQ ID NO: 18 - protein, 485 aa, Bacillus sp. NCIB 12513
  MISC_FEATURE: corresponding to SEQ ID NO 2 of WO 1996/023873
HHNGTNGTMMQYFEWHLPNDGNHWNRLRDDASNLRNRGITAIWIPPAWKGTSQNDVGYGAYDLYDLGEFNQKGTVRTKYGTRSQLESAIHALKNNGVQVYGDVVMNHKGGADATENVLAVEVNPNNRNQEISGDYTIEAWTKFDFPGRGNTYSDFKWRWYHFDGVDWDQSRQFQNRIYKFRGDGKAWDWEVDSENGNYDYLMYADVDMDHPEVVNELRRWGEWYTNTLNLDGFRIDAVKHIKYSFTRDWLTHVRNATGKEMFAVAEFWKNDLGALENYLNKTNWNHSVFDVPLHYNLYNASNSGGNYDMAKLLNGTVVQKHPMHAVTFVDNHDSQPGESLESFVQEWFKPLAYALILTREQGYPSVFYGDYYGIPTHSVPAMKAKIDPILEARQNFAYGTQHDYFDHHNIIGWTREGNTTHPNSGLATIMSDGPGGEKWMYVGQNKAGQVWHDITGNKPGTVTINADGWANFSVNGGSVSIWVKR

SEQ ID NO: 19 - protein, 485 aa, Bacillus sp. #707; identical to SEQ ID NO: 16
  MISC_FEATURE: corresponding to SEQ ID NO 7 of WO 1996/023873

SEQ ID NO: 20 - protein, 485 aa, Bacillus sp. A 7-7 (DSM 12368)
  MISC_FEATURE: corresponding to SEQ ID NO 2 of WO 2008/153815
HHNGTNGTMMQYFEWYLPNDGNHWNRLRSDASNLKDKGITAVWIPPAWKGASQNDVGYGAYDLYDLGEFNQKGTVRTKYGTRNQLQAAVTALKSNGIQVYGDVVMNHKGGADATEWVRAVEVNPSNRNQEVSGDYTIEAWTKFDFPGRGNTHSNFKWRWYHFDGVDWDQSRQLQNRIYKFRGDGKGWDWEVDTENGNYDYLMYADIDMDHPEVVNELRNWGVWYTNTLGLDGFRIDAVKHIKYSFTRDWLTHVRNTTGKNMFAVAEFWKNDIGAIENYLSKTNWNHSVFDVPLHYNLYNASRSGGNYDMRQIFNGTVVQRHPTHAVTFVDNHDSQPEEALESFVEEWFKPLAYALTLTRDQGYPSVFYGDYYGIPTHGVPAMKSKIDPILEARQKYAYGKQNDYLDHHNMIGWTREGNTAHPNSGLATIMSDGPGGNKWMYVGRNKAGQVWRDITGNRSGTVTINADGWGNFSVNGGSVSIWVNN

SEQ ID NO: 21 - protein, 483 aa, Bacillus amyloliquefaciens
  MISC_FEATURE: corresponding to SEQ ID NO 10 of WO 2001/066712
VNGTLMQYFEWYTPNDGQHWKRLQNDAEHLSDIGITAVWIPPAYKGLSQSDNGYGPYDLYDLGEFQQKGTVRTKYGTKSELQDAIGSLHSRNVQVYGDVVLNHKAGADATEDVTAVEVNPANRNQETSEEYQIKAWTDFRFPGRGNTYSDFKWHWYHFDGADWDESRKISRIFKFRGEGKAWDWEVSSENGNYDYLMYADVDYDHPDVVAETKKWGIWYANELSLDGFRIDAAKHIKFSFLRDWVQAVRQATGKEMFTVAEYWQNNAGKLENYLNKTSFNQSVFDVPLHFNLQAASSQGGGYDMRRLLDGTVVSRHPEKAVTFVENHDTQPGQSLESTVQTWFKPLAYAFILTRESGYPQVFYGDMYGTKGTSPKEIPSLKDNIEPILKARKEYAYGPQHDYIDHPDVIGWTREGDSSAAKSGLAALITDGPGGSKRMYAGLKNAGETWYDITGNRSDTVKIGSDGWGEFHVNDGSVSIYVQK

SEQ ID NO: 22 - protein, 484 aa, Bacillus sp. TS-23
  MISC_FEATURE: corresponding to SEQ ID NO 2 of WO 2009/061380
NTAPINETMMQYFEWDLPNDGTLWTKVKNEAANLSSLGITALWLPPAYKGTSQSDVGYGVYDLYDLGEFNQKGTIRTKYGTKTQYIQAIQAAKAAGMQVYADVVFNHKAGADGTEFVDAVEVDPSNRNQETSGTYQIQAWTKFDFPGRGNTYSSFKWRWYHFDGTDWDESRKLNRIYKFRSTGKAWDWEVDTENGNYDYLMFADLDMDHPEVVTELKNWGTWYVNTTNIDGFRLDAVKHIKYSFFPDWLTYVRNQTGKNLFAVGEFWSYDVNKLHNYITKTNGSMSLFDAPLHNNFYTASKSSGYFDMRYLLNNTLMKDQPSLAVTLVDNHDTQPGQSLQSWVEPWFKPLAYAFILTRQEGYPCVFYGDYYGIPKYNIPGLKSKIDPLLIARRDYAYGTQRDYIDHQDIIGWTREGIDTKPNSGLAALITDGPGGSKWMYVGKKHAGKVFYDLTGNRSDTVTINADGWGEFKVNGGSVSIWVAK

SEQ ID NO: 23 - protein, 214 aa, Bacillus amyloliquefaciens
  MISC_FEATURE: corresponding to SEQ ID NO 3 in WO 2015/144824
  mat_peptide (1)..(214)
QTGGSFFEPFNSYNSGLWQKANGYSNGDMFNCTWRANNVSMTSSGEMRLALTSPSYNKFDCGENRSVQTYGYGLYEVRMKPAKNTGIVSSFFTYTGPTDGTPWDEIDIEFLGKDTTKVQFNYYTNGAGNHEKVADLGFDATNAYHTYAFDWQPNSIKWYVDGQLKHTATSQIPTNPGKIMMNLWNGIGVDDWLGSYNGVNPLYAHYDWVRYTKK

SEQ ID NO: 24 - protein, 214 aa, Bacillus subtilis
  mat_peptide (1)..(214)
  MISC_FEATURE (1)..(214): corresponding to SEQ ID NO 4 in WO 2015/144824
QTGGSFFDPFNGYNSGFWQKADGYSNGNMFNCTWRANNVSMTSLGEMRLALTSPAYNKFDCGENRSVQTYGYGLYEVRMKPAKNTGIVSSFFTYTGPTDGTPWDEIDIEFLGKDTTKVQFNYYTNGAGNHEKIVDLGFDAANAYHTYAFDWQPNSIKWYVDGQLKHTATNQIPTTPGKIMMNLWNGTGVDEWLGSYNGVNPLYAHYDWVRYTKK

SEQ ID NO: 25 - protein, 245 aa, Bacillus akibai
  mat_peptide (1)..(245)
AERPIGTTFVETFESYDSERWSKAGVWTNGQMFNATWYPEQVTFSDGKMKLQIDKEDNETASPPYKAGELRTNDFYHYGLFEVSMKPAKSTGTVSSFFTYTGPWDWDNDPWDEIDIEFLGKDTTKIQFNYFTNGVGGNEHYHELGFDAADDFNTYAFEWRPESIRWFVNGELVHTATENIPQTPQKIMMNLWPGIGVDGWTGRFNGEDTPVVTQYDWVKYTPLEELGCYNEKNNKYKKCKKTKVK

SEQ ID NO: 26 - protein, 222 aa, Bacillus agaradhaerens
  mat_peptide (1)..(222)
HNPVTDEEVYHSFNSHDWQNWNMSDGWKNDDYFFGCHWSQNRVNFYGGQMELSLRTNYSYAPPYNYECAEYTTNNFYGYGLYEVSMKPAKVSGVISSFFTYTGPSYNGAPWDEIDIEFLGNDTTKVQFNYYTDGVGGNEILYDLGFDAADSYNTYAFDWQENYINWYVNGQLVATATENIPSNPSKIMMNIWNTYGIDEWAGRYYGEDANASYNWVRYTPNR

SEQ ID NO: 27 - protein, 351 aa, Bacillus sp-62449
  mat_peptide (1)..(351)
YNPVTEDELYHSFDSHDARNWQISDGWRNGDDFFGCHWSQNRVNFNRGEMELSLRTNYSYSAPYNYECAEYATSNFYGYGLYEVSMKPANVSGVISSFFTYTGPSYNGAPWDEIDIEFLGNDTTKVQFNYYTNGVGGNEIIYDLGFDAANSFNTYAFDWQENYISWYVNGNLVATATENIPSNPSKIMMNVWNTYGIDEWAGAYGGEAANATYEWVRYTPNNGNTTPSTAPDFQLQACDYSDSSGITSWSCGVGTFHSSNWIKFDSVDLSTGYNAFAVSYTSPGSGSFDIRLGSPHGQRIGTVNYGATGGWSNYEWSGTPSLDVTVRGAHDIYIVATSGAANLREFWFKNE

SEQ ID NO: 28 - protein, 214 aa, Bacillus mojavensis
  mat_peptide (1)..(214)
QTGGSFFDPFNGYNSGFWQKANGYSNGNMFNCTWRANNVSMTSLGEMRLALTSPSYNKFDCGENRSVQTYGYGLYEVRMKPAKNVGIVSSFFTYTGPTDGTPWDEIDIEFLGKDTTKVQFNYYTNGVGNHEKLVDLGFDAANAYHTYAFDWQPNSIKWYVDGQLKHTATSQIPTTPGKIMMNLWNGTGVDEWLGSYNGVTPLYAHYDWVRYTKK

SEQ ID NO: 29 - protein, 485 aa, Cytophaga sp.
  mat_peptide (1)..(485)
AATNGTMMQYFEWYVPNDGQQWNRLRTDAPYLSSVGITAVWTPPAYKGTSQADVGYGPYDLYDLGEFNQKGTVRTKYGTKGELKSAVNTLHSNGIQVYGDVVMNHKAGADYTENVTAVEVNPSNRNQETSGEYNIQAWTGFNFPGRGTTYSNFKWQWFHFDGTDWDQSRSLSRIFKFRGTGKAWDWEVSSENGNYDYLMYADIDYDHPDVVNEMKKWGVWYANEVGLDGYRLDAVKHIKFSFLKDWVDNARAATGKEMFTVGEYWQNDLGALNNYLAKVNYNQSLFDAPLHYNFYAASTGGGYYDMRNILNNTLVASNPTKAVTLVENHDTQPGQSLESTVQPWFKPLAYAFILTRSGGYPSVFYGDMYGTKGTTTREIPALKSKIEPLLKARKDYAYGTQRDYIDNPDVIGWTREGDSTKAKSGLATVITDGPGGSKRMYVGTSNAGEIWYDLTGNRTDKITIGSDGYATFPVNGGSVSVWVQQ

(19) United States
(12) Patent Application Publication
(10) Pub. No.: US 2021/0171927 A1
(43) Pub. Date: Jun. 10, 2021

(54) BETA-GLUCANASE VARIANTS AND POLYNUCLEOTIDES ENCODING SAME

(71) Applicant: NOVOZYMES A/S, Bagsvard (DK)

(72) Inventors: Carsten Andersen, Vaerloese (DK); Iben Damager, Vaerloese (DK); Morten Gjermansen, Greve (DK)

(73) Assignee: NOVOZYMES A/S, Bagsvaerd (DK)

(21) Appl. No.: 16/065,261

(22) PCT Filed: Jan. 27, 2017
(86) PCT No.: PCT/EP2017/051791

§ 371 Date: Jun. 22, 2018

(30) Foreign Application Priority Data
Jan. 29, 2016 (EP) 16153419.3

(51) Int. Cl. C12N 9/42; C11D 3/386

(52) U.S. Cl. CPC C12N 9/2448; C11D 3/38636

(57) ABSTRACT

The present invention relates to beta-glucanase variants. The present invention also relates to polynucleotides encoding the variants; nucleic acid constructs, vectors, and host cells comprising the polynucleotides; and methods of using the variants.

### REFERENCE TO A SEQUENCE LISTING

This application contains a Sequence Listing in computer readable form, which is incorporated herein by reference.

### BACKGROUND OF THE INVENTION

### Field of the Invention

The present invention relates to beta-glucanase variants, polynucleotides encoding the variants, methods of producing the variants, and methods of using the variants.

### Description of the Related Art

Beta-glucans are polysaccharides consisting of glucose units linked by beta-glycosidic bonds. Cellulose is one type of beta-glucan, in which all of the glucose units are linked by beta-1,4-glucosidic bonds. This feature results in the formation of insoluble cellulose micro-fibrils. Enzymatic hydrolysis of cellulose to glucose requires the use of endo beta-glucanases (e.g. EC 3.2.1.4), cellobiohydrolases (e.g. EC 3.2.1.91) and beta-glucosidases (e.g. EC 3.2.1.21).

Beta-glucans can also be linked by beta-1,3-glucosidic bonds (e.g., as found in the cell walls of baker's yeast, Saccharomyces cerevisiae), beta-1,6-glucosidic bonds as well as combinations of beta-1,3-, beta-1,4- and beta-1,6-glucosidic bonds. The combination of beta-1,3- and beta-1,4-glucosidic bonds can be found, e.g. in the soluble fibre from cereals such as oats and barley. A subgroup of beta-glucanases, also known as a licheninases (or lichenases) (EC 3.2.1.73), can be used to catalyse the hydrolysis of the beta-1,4-glucosidic bonds to give beta-glucans. Licheninases (or lichenases) (e.g. EC 3.2.1.73) hydrolyse (1,4)-beta-D-glucosidic linkages in beta-D-glucans containing (1,3)- and (1,4)-bonds and can act on lichenin and cereal beta-D-glucans, but not on beta-D-glucans containing only 1,3- or 1,4-bonds. Other beta-glucanases (e.g. EC 3.2.1.4) can, for example, perform endohydrolysis of (1,4)-beta-D-glucosidic linkages in cellulose, lichenin and cereal beta-D-glucans and will also hydrolyze 1,4-linkages in beta-D-glucans containing 1,3-linkages. The removal of cereal stains as oat and barley containing stains in dish wash and laundry is a recognised problem, and there is a considerable interest in finding enzymes that can degrade the beta-glucans found therein. Various Bacillus species like, e.g. B. amyloliquefaciens, express beta-glucanases, but these enzymes are generally not very suitable for alkaline applications (e.g. at pH 7.5 or above) and/or are sensitive to bleaching agents present in powder and ADW detergents.

The present invention relates to polypeptides of glycoside hydrolyase family 16 (GH16) having beta-glucanase activity (e.g. comprising or consisting of licheninase (EC 3.2.1.73) activity) and polynucleotides encoding said polypeptides, which are highly active in degrading different types of beta-glucans (e.g. beta-D-glucans, beta-1,3-1,4 glucans, mix-linkage beta-glucans, barley beta-glucans and oatmeal beta-glucans), e.g. under alkaline conditions (e.g. at pH 7.5 or above) and/or in the presence of bleaching agents, and therefore could be used in the aforementioned applications, e.g. in cleaning or detergent applications and processes such as cleaning hard-surfaces, dish wash and laundering. The existing products comprising beta-glucanases are sensitive to bleaching agents present in powder and ADW detergents and/or have very low effect on this type of beta-glucan as their main enzymatic substrate is cellulose.

The present invention relates to variants of beta-glucanases with improved properties compared to their parents (e.g. improved stability in the presence of bleaching agents and/or improved stability under alkaline conditions) and variants of beta-glucanases without cellulase activity (e.g. not having endo-cellulase activity on β-1,4 linkages between D-glucose units) (e.g. EC 3.2.1.73). A difference between use of cellulases and lichenases on textile in laundry is that lichenases do not degrade fibers of the textile.

### SUMMARY OF THE INVENTION

In one aspect the present invention relates to a variant of a parent beta-glucanase, the variant comprising a substitution at one or more positions corresponding to positions 33 (e.g., F33) and 188 (e.g., M188) of the mature polypeptide of SEQ ID NO: 26 using the numbering of SEQ ID NO: 26, wherein the variant has beta-glucanase activity and wherein the variant has at least 60%, e.g., at least 61%, at least 62%, at least 63%, at least 64%, at least 65%, at least 66%, at least 67%, at least 68%, at least 69%, at least 70%, at least 71%, at least 72%, at least 73%, at least 74%, at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 95.5%, at least 96%, at least 96.5%, at least 97%, at least 97.5%, at least 98%, at least 98.5%, at least 99%, or at least 99.5%, but less than 100% sequence identity to the mature polypeptide of any of: SEQ ID NO: 26, SEQ ID NO: 27, SEQ ID NO: 25, and SEQ ID NO: 28.

In a further aspect the present invention relates to use of a beta-glucanase variant of the invention or a composition comprising a beta-glucanase variant of the invention in a cleaning process such as laundry or hard surface cleaning including dish wash; optionally said use is carried out under alkaline conditions having pH 7.5 (or above) and/or in the presence of a bleaching agent. In a still further aspect the present invention also relates to compositions comprising a variant of the present invention and uses of variants of the present invention for/in degrading a beta-glucan (e.g. beta-D-glucan, beta-1,3-1,4 glucan, a mix-linkage beta-glucan, barley beta-glucan, oatmeal beta-glucan), controlling the viscosity of fluids (e.g. drilling fluids), washing or cleaning a textile and/or a hard surface; methods for degrading beta-glucan comprising applying a composition comprising a variant of the present invention to a beta-glucan. In a further aspect a beta-glucanase variant of the present invention is a lichenase variant. In a further aspect a difference between use of known cellulases and lichenase variants of the present invention on textile in laundry is that lichenase variants do not degrade fibers of the textile. The present invention also relates to methods of laundering fabrics or textiles or hard surface cleaning including automated dish wash (ADW) and hand dish wash (HDW) using a variant or a composition (e.g. cleaning or detergent composition) of the invention. The present invention also relates to polynucleotides encoding variants of the invention; nucleic acid constructs; recombinant expression vectors; recombinant host cells comprising said polynucleotides; and methods of producing variants of the invention.

### BRIEF DESCRIPTION OF THE FIGURES

FIG. 1 shows multiple alignment of beta-glucanases having the following sequences: SEQ ID NO: 23, SEQ ID NO: 24, SEQ ID NO: 25, SEQ ID NO: 26, SEQ ID NO: 27 and SEQ ID NO: 28.

### OVERVIEW OF SEQUENCE LISTING

SEQ ID NO: 1 is the DNA sequence of the beta-glucanase as isolated from a strain of a Bacillus sp-62499.

SEQ ID NO: 2 is the amino acid sequence of the beta-glucanase as automatically deduced from SEQ ID NO: 1.

SEQ ID NO: 3 is the amino acid sequence of the beta-glucanase as deduced from SEQ ID NO: 1 taking into account that the first amino acid (position −28) in the polypeptide shown in

SEQ ID NO: 2 and encoded by the polynucleotide shown in SEQ ID NO:1 should be Met, not Val. When the first codon is gtg a Met is inserted though gtg normally codes for V.

SEQ ID NO: 4 is the DNA sequence of the beta-glucanase as isolated from a strain of a Bacillus akibai.

SEQ ID NO: 5 is the amino acid sequence of the beta-glucanase as deduced from SEQ ID NO: 4.

SEQ ID NO: 6 is the DNA sequence of the beta-glucanase as isolated from a strain of a Bacillus agaradhaerens.

SEQ ID NO: 7 is the amino acid sequence of the beta-glucanase as deduced from SEQ ID NO: 6.

SEQ ID NO: 8 is the DNA sequence of the beta-glucanase as isolated from a strain of a Bacillus mojavensis.

SEQ ID NO: 9 is the amino acid sequence of the beta-glucanase as deduced from SEQ ID NO: 8.

SEQ ID NO: 10 is a polypeptide secretion signal Bacillus clausii.

SEQ ID NO: 11 is an artificial N-terminal poly-histidine affinity purification tag sequence.

SEQ ID NO: 12 is alpha-amylase protein sequence from Bacillus sp. (commercially sold by Novozymes A/S under the tradename Stainzyme).

SEQ ID NO: 13 is a polypeptide corresponding to SEQ ID NO: 2 of WO 95/10603.

SEQ ID NO: 14 is a polypeptide corresponding to SEQ ID NO: 6 in WO 02/010355.

SEQ ID NO: 15 is a polypeptide corresponding to a hybrid polypeptide comprising residues 1-33 of SEQ ID NO: 6 of WO 2006/066594 and residues 36-483 of SEQ ID NO: 4 of WO 2006/066594.

SEQ ID NO: 16 is a polypeptide corresponding to SEQ ID NO: 6 of WO 02/019467.

SEQ ID NO: 17, SEQ ID NO: 18 and SEQ ID NO: 19 are polypeptides respectively corresponding to SEQ ID NO: 1, SEQ ID NO: 2 or SEQ ID NO: 7 of WO 96/023873.

SEQ ID NO: 20 is a polypeptide corresponding to SEQ ID NO: 2 of WO 08/153815.

SEQ ID NO: 21 is a polypeptide corresponding to SEQ ID NO: 10 of WO 01/66712.

SEQ ID NO: 22 is a polypeptide corresponding to SEQ ID NO: 2 of WO 09/061380.

SEQ ID NO: 23 is the mature polypeptide of the beta-glucanase from a strain of Bacillus amyloliquefaciens corresponding to SEQ ID NO: 3 in WO 2015/144824.

SEQ ID NO: 24 is the mature polypeptide of the beta-glucanase from a strain of Bacillus subtilis corresponding to SEQ ID NO: 4 in WO 2015/144824.

SEQ ID NO: 25 is the mature polypeptide of SEQ ID NO: 5 (i.e. corresponding to amino acids 1 to 245 of SEQ ID NO: 5).

SEQ ID NO: 26 is the mature polypeptide of SEQ ID NO: 7 (i.e. corresponding to amino acids 1 to 222 of SEQ ID NO: 7).

SEQ ID NO: 27 is the mature polypeptide of SEQ ID NO: 3 and SEQ ID NO: 2 (i.e. corresponding to amino acids 1 to 351 of SEQ ID NO: 3, amino acids 1 to 351 of SEQ ID NO: 2).

SEQ ID NO: 28 is the mature polypeptide of SEQ ID NO: 9 (i.e. corresponding to amino acids 1 to 214 of SEQ ID NO: 9).

SEQ ID NO: 29 is the amino acid sequence of a mature cytophaga alpha-amylase.

### Definitions

Synergistic effect: The term “synergistic effect” means a cooperative action of polypeptides such that a total combined effect of said polypeptides is greater than the sum of the individual enzymatic effects of said polypeptides. Non-limiting examples of synergistic effect include REM synergistic effect of a beta-glucanase polypeptide of the invention and one or more alpha-amylase.

REM synergistic effect: REM synergistic effect of polypeptides as used herein can be measured based on the analysis of stain removal carried out by using any suitable wash performance methodology (e.g. Wascator bottle wash method). A preferred method for determining the REM synergistic effect is disclosed in Example 7.

Beta-glucanase: The term “beta-glucanase” as used herein means an endo beta-1,4-glucanase activity (e.g. endo-1,4-β-D-glucanase) that catalyzes the hydrolyses of a beta-1,4-bonds connecting two glucosyl residues in a beta-glucan. Non-limiting examples of beta-glucanases as defined herein include cellulases (e.g. EC 3.2.1.4, e.g. having endo-cellulase activity on β-1,4 linkages between D-glucose units and licheninases (or lichenases) (e.g. EC 3.2.1.73) hydrolysing (1,4)-beta-D-glucosidic linkages in beta-D-glucans containing (1,3)- and (1,4)-bonds. Beta-glucanases (e.g. EC 3.2.1.4) can, for example, perform endohydrolysis of (1,4)-beta-D-glucosidic linkages in cellulose, lichenin and cereal beta-D-glucans and will also hydrolyze 1,4-linkages in beta-D-glucans containing 1,3-linkages. For purposes of the present invention, beta-glucanase activity is determined according to the procedure described in the Examples. In one aspect of the invention, the polypeptides of the present invention (e.g beta-glucanase variants) have at least 20%, e.g., at least 40%, at least 50%, at least 60%, at least 70%, at least 80%, at least 90%, at least 95%, or at least 100% of the beta-glucanase activity of the polypeptide having the sequence selected from the group consisting of: SEQ ID NO: 7, SEQ ID NO: 2, SEQ ID NO: 3, SEQ ID NO: 5, SEQ ID NO: 9, SEQ ID NO: 25, SEQ ID NO: 26, SEQ ID NO: 27, SEQ ID NO: 28. Beta-glucanase activity can suitably be measured using barley beta-glucan as substrate. A preferred assay for determining beta-glucanase activity is disclosed in Example 1 (AZCL-Barley beta-glucan assay). A further subgroup of beta-glucanases as defined herein, also known as a licheninases (or lichenases) (e.g. EC 3.2.1.73), can also be used to catalyse the hydrolysis of the beta-1,4-glucosidic bonds to give beta-glucans. Licheninases (or lichenases) (e.g. EC 3.2.1.73) hydrolyse (1,4)-beta-D-glucosidic linkages in beta-D-glucans containing (1,3)-and (1,4)-bonds and can act on lichenin and cereal beta-D-glucans, but not on beta-D-glucans containing only 1,3- or 1,4-bonds. As used herein the term “beta-glucanase activity” comprises licheninase (or lichenases) (e.g. EC 3.2.1.73) activity.

Beta-glucan: The term “beta-glucan” as used herein means a polysaccharide that only contains glucose as structural components, and in which the glucose units are linked by beta-glycosidic bonds. Non-limiting examples of beta-glucans include beta-D-glucans, beta-1,3-1,4 glucans, mix-linkage beta-glucans, barley beta-glucans, oatmeal beta-glucans.

Allelic variant: The term “allelic variant” means any of two or more alternative forms of a gene occupying the same chromosomal locus. Allelic variation arises naturally through mutation, and may result in polymorphism within populations. Gene mutations can be silent (no change in the encoded polypeptide) or may encode polypeptides having altered amino acid sequences. An allelic variant of a polypeptide is a polypeptide encoded by an allelic variant of a gene.

Biofilm: The term “biofilm” means any group of microorganisms in which cells stick to each other on a surface, such as a textile, dishware or hard surface. These adherent cells are frequently embedded within a self-produced matrix of extracellular polymeric substance (EPS). Biofilm EPS is a polymeric conglomeration generally composed of extracellular DNA, proteins, and polysaccharides. Biofilms may form on living or non-living surfaces. The microbial cells growing in a biofilm are physiologically distinct from planktonic cells of the same organism, which, by contrast, are single-cells that may float or swim in a liquid medium.

Bacteria living in a biofilm usually have significantly different properties from free-floating bacteria of the same species, as the dense and protected environment of the film allows them to cooperate and interact in various ways. One effect of this environment is increased resistance to detergents and antibiotics, as the dense extracellular matrix and the outer layer of cells protect the interior of the community.

On laundry biofilm producing bacteria can be found among the following species: Acinetobacter sp., Aeromicrobium sp., Brevundimonas sp., Microbacterium sp., Micrococcus luteus, Pseudomonas sp., Staphylococcus epidermidis, and Stenotrophomonas sp.

Carbohydrate binding module: The term “carbohydrate binding module” means the region within a carbohydrate-active enzyme that provides carbohydrate-binding activity (Boraston et al., 2004, Biochem. J. 383: 769-781). A majority of known carbohydrate binding modules (CBMs) are contiguous amino acid sequences with a discrete fold. The carbohydrate binding module (CBM) is typically found either at the N-terminal or at the C-terminal extremity of an enzyme. Some CBMs are known to have specificity for cellulose.

Catalytic domain: The term “catalytic domain” means the region of an enzyme containing the catalytic machinery of the enzyme.

cDNA: The term “cDNA” means a DNA molecule that can be prepared by reverse transcription from a mature, spliced, mRNA molecule obtained from a eukaryotic or prokaryotic cell. cDNA lacks intron sequences that may be present in the corresponding genomic DNA. The initial, primary RNA transcript is a precursor to mRNA that is processed through a series of steps, including splicing, before appearing as mature spliced mRNA.

Cellulolytic enzyme or cellulase: The term “cellulolytic enzyme” or “cellulase” means one or more (e.g., several) enzymes that hydrolyze a cellulosic material. Such enzymes include endoglucanase(s) (e.g. EC 3.2.1.4), cellobiohydrolase(s), beta-glucosidase(s), or combinations thereof. The two basic approaches for measuring cellulolytic enzyme activity include: (1) measuring the total cellulolytic enzyme activity, and (2) measuring the individual cellulolytic enzyme activities (endoglucanases, cellobiohydrolases, and beta-glucosidases) as reviewed in Zhang et al., 2006, Biotechnology Advances 24: 452-481. Total cellulolytic enzyme activity may be measured using insoluble substrates, including Whatman No1 filter paper, microcrystalline cellulose, bacterial cellulose, algal cellulose, cotton, pretreated lignocellulose, etc. The most common total cellulolytic activity assay is the filter paper assay using Whatman No1 filter paper as the substrate. The assay was established by the International Union of Pure and Applied Chemistry (IUPAC) (Ghose, 1987, Pure Appl. Chem. 59: 257-68).

Cellulolytic enzyme activity can be determined by measuring the increase in production/release of sugars during hydrolysis of a cellulosic material by cellulolytic enzyme(s) under the following conditions: 1-50 mg of cellulolytic enzyme protein/g of cellulose in pretreated corn stover (PCS) (or other pretreated cellulosic material) for 3-7 days at a suitable temperature such as 40° C.-80° C., e.g., 50° C., 55° C., 60° C., 65° C., or 70° C., and a suitable pH such as 4-9, e.g., 5.0, 5.5, 6.0, 6.5, or 7.0, compared to a control hydrolysis without addition of cellulolytic enzyme protein. Typical conditions are 1 ml reactions, washed or unwashed PCS, 5% insoluble solids (dry weight), 50 mM sodium acetate pH 5, 1 mM MnSO₄, 50° C., 55° C., or 60° C., 72 hours, sugar analysis by AMINEX® HPX-87H column (Bio-Rad Laboratories, Inc., Hercules, Calif., USA).

Cellulosic material: The term “cellulosic material” means any material containing cellulose. The predominant polysaccharide in the primary cell wall of biomass is cellulose, the second most abundant is hemicellulose, and the third is pectin. The secondary cell wall, produced after the cell has stopped growing, also contains polysaccharides and is strengthened by polymeric lignin covalently cross-linked to hemicellulose. Cellulose is a homopolymer of anhydrocellobiose and thus a linear beta-(1-4)-D-glucan, while hemicelluloses include a variety of compounds, such as xylans, xyloglucans, arabinoxylans, and mannans in complex branched structures with a spectrum of substituents. Although generally polymorphous, cellulose is found in plant tissue primarily as an insoluble crystalline matrix of parallel glucan chains. Hemicelluloses usually hydrogen bond to cellulose, as well as to other hemicelluloses, which help stabilize the cell wall matrix.

Cellulose is generally found, for example, in the stems, leaves, hulls, husks, and cobs of plants or leaves, branches, and wood of trees. The cellulosic material can be, but is not limited to, agricultural residue, herbaceous material (including energy crops), municipal solid waste, pulp and paper mill residue, waste paper, and wood (including forestry residue) (see, for example, Wiselogel et al., 1995, in Handbook on Bioethanol (Charles E. Wyman, editor), pp. 105-118, Taylor & Francis, Washington D.C.; Wyman, 1994, Bioresource Technology50: 3-16; Lynd, 1990, Applied Biochemistry and Biotechnology 24/25: 695-719; Mosier et al., 1999, Recent Progress in Bioconversion of Lignocellulosics, in Advances in Biochemical Engineering/Biotechnology, T. Scheper, managing editor, Volume 65, pp. 23-40, Springer-Verlag, New York). It is understood herein that the cellulose may be in the form of lignocellulose, a plant cell wall material containing lignin, cellulose, and hemicellulose in a mixed matrix. In one aspect, the cellulosic material is any biomass material. In another aspect, the cellulosic material is lignocellulose, which comprises cellulose, hemicelluloses, and lignin.

In an embodiment, the cellulosic material is agricultural residue, herbaceous material (including energy crops), municipal solid waste, pulp and paper mill residue, waste paper, or wood (including forestry residue).

In another embodiment, the cellulosic material is arundo, bagasse, bamboo, corn cob, corn fiber, corn stover, miscanthus, rice straw, switchgrass, or wheat straw.

In another embodiment, the cellulosic material is aspen, eucalyptus, fir, pine, poplar, spruce, or willow.

In another embodiment, the cellulosic material is algal cellulose, bacterial cellulose, cotton linter, filter paper, microcrystalline cellulose (e.g., AVICEL®), or phosphoric-acid treated cellulose.

In another embodiment, the cellulosic material is an aquatic biomass. As used herein the term “aquatic biomass” means biomass produced in an aquatic environment by a photosynthesis process. The aquatic biomass can be algae, emergent plants, floating-leaf plants, or submerged plants.

The cellulosic material may be used as is or may be subjected to pretreatment, using conventional methods known in the art, as described herein. In a preferred aspect, the cellulosic material is pretreated.

Coding sequence: The term “coding sequence” means a polynucleotide, which directly specifies the amino acid sequence of a polypeptide. The boundaries of the coding sequence are generally determined by an open reading frame, which begins with a start codon such as ATG, GTG, or TTG and ends with a stop codon such as TAA, TAG, or TGA. The coding sequence may be a genomic DNA, cDNA, synthetic DNA, or a combination thereof.

Control sequences: The term “control sequences” means nucleic acid sequences necessary for expression of a polynucleotide encoding a mature polypeptide of the present invention. Each control sequence may be native (i.e., from the same gene) or foreign (i.e., from a different gene) to the polynucleotide encoding the polypeptide or native or foreign to each other. Such control sequences include, but are not limited to, a leader, polyadenylation sequence, propeptide sequence, promoter, signal peptide sequence, and transcription terminator. At a minimum, the control sequences include a promoter, and transcriptional and translational stop signals. The control sequences may be provided with linkers for the purpose of introducing specific restriction sites facilitating ligation of the control sequences with the coding region of the polynucleotide encoding a polypeptide.

Detergent component: the term “detergent component” is defined herein to mean the types of chemicals which can be used in detergent compositions. Examples of detergent components are surfactants, hydrotropes, builders, co-builders, chelators or chelating agents, bleaching system or bleach components, polymers, fabric hueing agents, fabric conditioners, foam boosters, suds suppressors, dispersants, dye transfer inhibitors, fluorescent whitening agents, perfume, optical brighteners, bactericides, fungicides, soil suspending agents, soil release polymers, anti-redeposition agents, enzyme inhibitors or stabilizers, enzyme activators, antioxidants, and solubilizers. The detergent composition may comprise of one or more of any type of detergent component.

Detergent composition: the term “detergent composition” refers to compositions that find use in the removal of undesired compounds from items to be cleaned, such as textiles, dishes, and hard surfaces. The detergent composition may be used to e.g. clean textiles, dishes and hard surfaces for both household cleaning and industrial cleaning. The terms encompass any materials/compounds selected for the particular type of cleaning composition desired and the form of the product (e.g., liquid, gel, powder, granulate, paste, or spray compositions) and includes, but is not limited to, detergent compositions (e.g., liquid and/or solid laundry detergents and fine fabric detergents; hard surface cleaning formulations, such as for glass, wood, plastic, ceramic and metal counter tops and windows; carpet cleaners; oven cleaners; fabric fresheners; fabric softeners; and textile and laundry pre-spotters, as well as dish wash detergents). In addition to containing a variant of the invention (e.g. a GH16 beta-glucanase variant), the detergent formulation may contain one or more additional enzymes (such as amylases, proteases, peroxidases, cellulases, betaglucanases, xyloglucanases, hemicellulases, xanthanases, xanthan lyases, lipases, acyl transferases, phospholipases, esterases, laccases, catalases, aryl esterases, amylases, alpha-amylases, glucoamylases, cutinases, pectinases, pectate lyases, keratinases, reductases, oxidases, phenoloxidases, lipoxygenases, ligninases, carrageenases, pullulanases, tannases, arabinosidases, hyaluronidases, chondroitinases, xyloglucanases, xylanases, pectin acetyl esterases, polygalacturonases, rhamnogalacturonases, other endo-beta-mannanases, exo-beta-mannanases, pectin methylesterases, cellobiohydrolases, transglutaminases, and combinations thereof, or any mixture thereof), and/or components such as surfactants, builders, chelators or chelating agents, bleach system or bleach components, polymers, fabric conditioners, foam boosters, suds suppressors, dyes, perfume, tannish inhibitors, optical brighteners, bactericides, fungicides, soil suspending agents, anti corrosion agents, enzyme inhibitors or stabilizers, enzyme activators, transferase(s), hydrolytic enzymes, oxido reductases, bluing agents and fluorescent dyes, antioxidants, and solubilizers.

Dish wash: The term “dish wash” refers to all forms of washing dishes, e.g. by hand dish wash (HDW), automatic dish wash (ADW), professional cleaning of hard surfaces, or warewash. Washing dishes includes, but is not limited to, the cleaning of all forms of crockery such as plates, cups, glasses, bowls, all forms of cutlery such as spoons, knives, forks and serving utensils as well as ceramics, plastics, metals, china, glass and acrylics.

Dish washing composition: The term “dish washing composition” refers to all forms of compositions for cleaning hard surfaces. The present invention is not restricted to any particular type of dish wash composition or any particular detergent.

Expression: The term “expression” includes any step involved in the production of a polypeptide including, but not limited to, transcription, post-transcriptional modification, translation, post-translational modification, and secretion.

Expression vector: The term “expression vector” means a linear or circular DNA molecule that comprises a polynucleotide encoding a polypeptide and is operably linked to control sequences that provide for its expression.

Fragment: The term “fragment” means a polypeptide or a catalytic or carbohydrate binding module having one or more (e.g., several) amino acids absent from the amino and/or carboxyl terminus of a mature polypeptide or domain; wherein the fragment has beta-glucanase or carbohydrate binding activity. In one aspect, a fragment contains at least 340 amino acid residues, or at least 230 amino acid residues, or at least 210 amino acid residues or at least 200 amino acid residues, or at least 180 amino acid residues, wherein the fragment has beta-glucanase activity.

Hard surface cleaning: The term “Hard surface cleaning” is defined herein as cleaning of hard surfaces wherein hard surfaces may include floors, tables, walls, roofs etc. as well as surfaces of hard objects such as cars (car wash) and dishes (dish wash). Dish washing includes but are not limited to cleaning of plates, cups, glasses, bowls, and cutlery such as spoons, knives, forks, serving utensils, ceramics, plastics, metals, china, glass and acrylics.

Hemicellulolytic enzyme or hemicellulase: The term “hemicellulolytic enzyme” or “hemicellulase” means one or more (e.g., several) enzymes that hydrolyze a hemicellulosic material. See, for example, Shallom and Shoham, Current Opinion In Microbiology, 2003, 6(3): 219-228). Hemicellulases are key components in the degradation of plant biomass. Examples of hemicellulases include, but are not limited to, an acetylmannan esterase, an acetylxylan esterase, an arabinanase, an arabinofuranosidase, a coumaric acid esterase, a feruloyl esterase, a galactosidase, a glucuronidase, a glucuronoyl esterase, a mannanase, a mannosidase, a xylanase, and a xylosidase. The substrates for these enzymes, hemicelluloses, are a heterogeneous group of branched and linear polysaccharides that are bound via hydrogen bonds to the cellulose microfibrils in the plant cell wall, crosslinking them into a robust network. Hemicelluloses are also covalently attached to lignin, forming together with cellulose a highly complex structure. The variable structure and organization of hemicelluloses require the concerted action of many enzymes for its complete degradation. The catalytic modules of hemicellulases are either glycoside hydrolases (GHs) that hydrolyze glycosidic bonds, or carbohydrate esterases (CEs), which hydrolyze ester linkages of acetate or ferulic acid side groups. These catalytic modules, based on homology of their primary sequence, can be assigned into GH and CE families. Some families, with an overall similar fold, can be further grouped into clans, marked alphabetically (e.g., GH-A). A most informative and updated classification of these and other carbohydrate active enzymes is available in the Carbohydrate-Active Enzymes (CAZy) database. Hemicellulolytic enzyme activities can be measured according to Ghose and Bisaria, 1987, Pure & Appl. Chem. 59: 1739-1752, at a suitable temperature such as 40° C.-80° C., e.g., 50° C., 55° C., 60° C., 65° C., or 70° C., and a suitable pH such as 4-9, e.g., 5.0, 5.5, 6.0, 6.5, or 7.0.

Host cell: The term “host cell” means any cell type that is susceptible to transformation, transfection, transduction, or the like with a nucleic acid construct or expression vector comprising a polynucleotide of the present invention. The term “host cell” encompasses any progeny of a parent cell that is not identical to the parent cell due to mutations that occur during replication, as well as a recombinant host cell, an isolated host cell (e.g., an isolated recombinant host cell), an isolated host cell that is not a human embryonic stem cell.

Improved property: The term “improved property” means a characteristic associated with a variant that is improved compared to the parent. Such improved properties include, but are not limited to, catalytic efficiency, catalytic rate, chemical stability, oxidation stability, pH activity, pH stability, specific activity, stability under storage conditions, substrate binding, substrate cleavage, substrate specificity, substrate stability, surface properties, thermal activity, and thermostability. Preferably the improved property associated with a variant of the invention is an improved oxidation stability (e.g. in the presence of a bleaching agent) compared with the parent beta-glucanase.

Oxidation stability: The term “oxidation stability” means resistance or the degree of resistance to one or more of the following: i) the complete, net removal of one or more electrons from a molecular entity, ii) an increase in the oxidation number of any atom within any substrate, iii) gain of oxygen and/or loss of hydrogen of an organic substrate, e.g., polypeptide and iv) degradation in a bleach containing environment.

Isolated: The term “isolated” means a substance in a form or environment that does not occur in nature. Non-limiting examples of isolated substances include (1) any non-naturally occurring substance, (2) any substance including, but not limited to, any enzyme, variant, nucleic acid, protein, peptide or cofactor, that is at least partially removed from one or more or all of the naturally occurring constituents with which it is associated in nature; (3) any substance modified by the hand of man relative to that substance found in nature; or (4) any substance modified by increasing the amount of the substance relative to other components with which it is naturally associated (e.g., recombinant production in a host cell; multiple copies of a gene encoding the substance; and use of a stronger promoter than the promoter naturally associated with the gene encoding the substance). A fermentation broth produced by culturing a recombinant host cell expressing the polynucleotide of the invention will comprise the polypeptide of the invention in an isolated form.

Laundering: The term “laundering” relates to both household laundering and industrial laundering and means the process of treating textiles with a solution containing a cleaning or detergent composition of the present invention. The laundering process can for example be carried out using e.g. a household or an industrial washing machine or can be carried out by hand.

Lichenase activity: The term “lichenase activity” means enzymes that hydrolysis beta-1,3-1,4-glucans (e.g. EC 3.2.1.73).

Mature polypeptide: The term “mature polypeptide” means a polypeptide in its final form following translation and any post-translational modifications, such as N-terminal processing, C-terminal truncation, glycosylation, phosphorylation, etc. In one aspect, the mature polypeptide is selected from the group consisting of: amino acids 1 to 222 of SEQ ID NO: 7 (which is also designated as SEQ ID NO: 26), amino acids 1 to 351 of SEQ ID NO: 2 (which is also designated as SEQ ID NO: 27), amino acids 1 to 351 of SEQ ID NO: 3 (which is also designated as SEQ ID NO: 27), amino acids 1 to 245 of SEQ ID NO: 5 (which is also designated as SEQ ID NO: 25), amino acids 1 to 214 of SEQ ID NO: 9 (which is also designated as SEQ ID NO: 28). The amino acids −28 to −1 of SEQ ID NO: 2 predicts a signal peptide. The amino acids −28 to −1 of SEQ ID NO: 3 predicts a signal peptide. The amino acids −31 to −1 of SEQ ID NO: 5 predicts a signal peptide. The amino acids −15 to −1 of SEQ ID NO: 7 predicts a signal peptide. The amino acids −29 to −1 of SEQ ID NO: 9 predicts a signal peptide. Non-limiting examples of mature polypeptide further include: SEQ ID NO: 23, which is the mature polypeptide of the beta-glucanase from a strain of Bacillus amyloliquefaciens corresponding to SEQ ID NO: 3 in WO 2015/144824 and SEQ ID NO: 24 which is the mature polypeptide of the beta-glucanase from a strain of Bacillus subtilis corresponding to SEQ ID NO: 4 in WO 2015/144824.

It is known in the art that a host cell may produce a mixture of two of more different mature polypeptides (i.e., with a different C-terminal and/or N-terminal amino acid) expressed by the same polynucleotide. It is also known in the art that different host cells process polypeptides differently, and thus, one host cell expressing a polynucleotide may produce a different mature polypeptide (e.g., having a different C-terminal and/or N-terminal amino acid) as compared to another host cell expressing the same polynucleotide.

Mature polypeptide coding sequence: The term “mature polypeptide coding sequence” means a polynucleotide that encodes a mature polypeptide having beta-glucanase activity. In one aspect, the mature polypeptide coding sequence is selected from the group consisting of: nucleotides 85 to 1137 of SEQ ID NO: 1, nucleotides 94 to 828 of SEQ ID NO: 4, nucleotides 46 to 711 of SEQ ID NO: 6, nucleotides 88 to 729 of SEQ ID NO: 8. The nucleotides 1 to 84 of SEQ ID NO: 1 encode a signal peptide. The nucleotides 1 to 93 of SEQ ID NO: 4 encode a signal peptide. The nucleotides 1 to 45 of SEQ ID NO: 6 encode a signal peptide. The nucleotides 1 to 87 of SEQ ID NO: 8 encode a signal peptide.

Malodor: The term“malodor” is meant an odor which is not desired on clean items. The cleaned item should smell fresh and clean without malodors adhered to the item. One example of malodor is compounds with an unpleasant smell, which may be produced by microorganisms. Another example is sweat or body odor adhering to an item which has been in contact with humans or animals. Another example of malodor can be the smell from spices, for example curry or other exotic spices adhering to an item such as a piece of textile. One way of measuring the ability of an item to adhere malodor is by using the Malodor Assay.

Nucleic acid construct: The term “nucleic acid construct” means a nucleic acid molecule, either single- or double-stranded, which is isolated from a naturally occurring gene or is modified to contain segments of nucleic acids in a manner that would not otherwise exist in nature or which is synthetic, which comprises one or more control sequences.

Operably linked: The term “operably linked” means a configuration in which a control sequence is placed at an appropriate position relative to the coding sequence of a polynucleotide such that the control sequence directs expression of the coding sequence.

Parent or parent beta-glucanase: The term “parent” or “parent beta-glucanase” means a beta-glucanase to which an alteration is made to produce the enzyme variants of the present invention. In one aspect, the parent is a beta-glucanase having the identical amino acid sequence of the variant, but not having the alterations at one or more of the specified positions. It will be understood, that the expression “having identical amino acid sequence” relates to 100% sequence identity. The parent may be a naturally occurring (wild-type) polypeptide or a variant or fragment thereof. Non-limiting examples of the parent beta-glucanase include mature polypeptides selected from the group consisting of: amino acids 1 to 222 of SEQ ID NO: 7 (which is also designated as SEQ ID NO: 26), amino acids 1 to 351 of SEQ ID NO: 2 (which is also designated as SEQ ID NO: 27), amino acids 1 to 351 of SEQ ID NO: 3 (which is also designated as SEQ ID NO: 27), amino acids 1 to 245 of SEQ ID NO: 5 (which is also designated as SEQ ID NO: 25), amino acids 1 to 214 of SEQ ID NO: 9 (which is also designated as SEQ ID NO: 28).

Pretreated corn stover: The term “Pretreated Corn Stover” or “PCS” means a cellulosic material derived from corn stover by treatment with heat and dilute sulfuric acid, alkaline pretreatment, neutral pretreatment, or any pretreatment known in the art.

Sequence identity: The relatedness between two amino acid sequences or between two nucleotide sequences is described by the parameter “sequence identity”. For purposes of the present invention, the sequence identity between two amino acid sequences is determined using the Needleman-Wunsch algorithm (Needleman and Wunsch, 1970, J. Mol. Biol. 48: 443-453) as implemented in the Needle program of the EMBOSS package (EMBOSS: The European Molecular Biology Open Software Suite, Rice et al., 2000, Trends Genet. 16: 276-277), preferably version 5.0.0 or later. The parameters used are gap open penalty of 10, gap extension penalty of 0.5, and the EBLOSUM62 (EMBOSS version of BLOSUM62) substitution matrix. The output of Needle labeled “longest identity” (obtained using the—nobrief option) is used as the percent identity and is calculated as follows:

(Identical Residues×100)/(Length of Alignment−Total Number of Gaps in Alignment)

For purposes of the present invention, the sequence identity between two deoxyribonucleotide sequences is determined using the Needleman-Wunsch algorithm (Needleman and Wunsch, 1970, supra) as implemented in the Needle program of the EMBOSS package (EMBOSS: The European Molecular Biology Open Software Suite, Rice et al., 2000, supra), preferably version 5.0.0 or later. The parameters used are gap open penalty of 10, gap extension penalty of 0.5, and the EDNAFULL (EMBOSS version of NCBI NUC4.4) substitution matrix. The output of Needle labeled “longest identity” (obtained using the—nobrief option) is used as the percent identity and is calculated as follows:

(Identical Deoxyribonucleotides×100)/(Length of Alignment−Total Number of Gaps in Alignment)

Stringency conditions: The different stringency conditions are defined as follows.

The term “very low stringency conditions” means for probes of at least 100 nucleotides in length, prehybridization and hybridization at 42° C. in 5×SSPE, 0.3% SDS, 200 micrograms/ml sheared and denatured salmon sperm DNA, and 25% formamide, following standard Southern blotting procedures for 12 to 24 hours. The carrier material is finally washed three times each for 15 minutes using 1.6×SSC, 0.2% SDS at 60° C.

The term “low stringency conditions” means for probes of at least 100 nucleotides in length, prehybridization and hybridization at 42° C. in 5×SSPE, 0.3% SDS, 200 micrograms/ml sheared and denatured salmon sperm DNA, and 25% formamide, following standard Southern blotting procedures for 12 to 24 hours. The carrier material is finally washed three times each for 15 minutes using 0.8×SSC, 0.2% SDS at 60° C.

The term “medium stringency conditions” means for probes of at least 100 nucleotides in length, prehybridization and hybridization at 42° C. in 5×SSPE, 0.3% SDS, 200 micrograms/ml sheared and denatured salmon sperm DNA, and 35% formamide, following standard Southern blotting procedures for 12 to 24 hours. The carrier material is finally washed three times each for 15 minutes using 0.8×SSC, 0.2% SDS at 65° C.

The term “medium-high stringency conditions” means for probes of at least 100 nucleotides in length, prehybridization and hybridization at 42° C. in 5×SSPE, 0.3% SDS, 200 micrograms/ml sheared and denatured salmon sperm DNA, and 35% formamide, following standard Southern blotting procedures for 12 to 24 hours. The carrier material is finally washed three times each for 15 minutes using 0.4×SSC, 0.2% SDS at 65° C.

The term “high stringency conditions” means for probes of at least 100 nucleotides in length, prehybridization and hybridization at 42° C. in 5×SSPE, 0.3% SDS, 200 micrograms/ml sheared and denatured salmon sperm DNA, and 50% formamide, following standard Southern blotting procedures for 12 to 24 hours. The carrier material is finally washed three times each for 15 minutes using 0.2×SSC, 0.2% SDS at 65° C.

The term “very high stringency conditions” means for probes of at least 100 nucleotides in length, prehybridization and hybridization at 42° C. in 5×SSPE, 0.3% SDS, 200 micrograms/ml sheared and denatured salmon sperm DNA, and 50% formamide, following standard Southern blotting procedures for 12 to 24 hours. The carrier material is finally washed three times each for 15 minutes using 0.2×SSC, 0.2% SDS at 70° C.

Subsequence: The term “subsequence” means a polynucleotide having one or more (e.g., several) nucleotides absent from the 5′ and/or 3′ end of a mature polypeptide coding sequence; wherein the subsequence encodes a fragment having beta-glucanase activity. In one aspect, a subsequence contains at least 2085 nucleotides of SEQ ID NO: 1 or the cDNA sequence thereof, at least 2070 nucleotides of SEQ ID NO: 1 or the cDNA sequence thereof, or 2055 nucleotides of SEQ ID NO: 1 or the cDNA sequence thereof).

Textile: The term “textile” means any textile material including yarns, yarn intermediates, fibers, non-woven materials, natural materials, synthetic materials, and any other textile material, fabrics made of these materials and products made from fabrics (e.g., garments and other articles). The textile or fabric may be in the form of knits, wovens, denims, non-wovens, felts, yarns, and towelling. The textile may be cellulose based such as natural cellulosics, including cotton, flax/linen, jute, ramie, sisal or coir or manmade cellulosics (e.g. originating from wood pulp) including viscose/rayon, ramie, cellulose acetate fibers (tricell), lyocell or blends thereof. The textile or fabric may also be non-cellulose based such as natural polyamides including wool, camel, cashmere, mohair, rabit and silk or synthetic polymer such as nylon, aramid, polyester, acrylic, polypropylen and spandex/elastane, or blends thereof as well as blend of cellulose based and non-cellulose based fibers. Examples of blends are blends of cotton and/or rayon/viscose with one or more companion material such as wool, synthetic fibers (e.g. polyamide fibers, acrylic fibers, polyester fibers, polyvinyl alcohol fibers, polyvinyl chloride fibers, polyurethane fibers, polyurea fibers, aramid fibers), and cellulose-containing fibers (e.g. rayon/viscose, ramie, flax/linen, jute, cellulose acetate fibers, lyocell). Fabric may be conventional washable laundry, for example stained household laundry. When the term fabric or garment is used it is intended to include the broader term textiles as well.

Variant: The term “variant” means a polypeptide having beta-glucanase activity comprising an alteration, i.e., a substitution, insertion, and/or deletion of one or more (several) amino acid residues at one or more (several) positions. A substitution means a replacement of an amino acid occupying a position with a different amino acid; a deletion means removal of an amino acid occupying a position; and an insertion means adding 1-3 amino acids adjacent to an amino acid occupying a position. The variants of the present invention have at least 20%, e.g., at least 40%, at least 50%, at least 60%, at least 70%, at least 80%, at least 90%, at least 95%, or at least 100% of the beta-glucanase activity of the polypeptide of sequence selected from the group consisting of: SEQ ID NO: 7, SEQ ID NO: 2, SEQ ID NO: 3, SEQ ID NO: 5, SEQ ID NO: 9 or the mature polypeptide of sequence selected from the group consisting of: SEQ ID NO: 7, SEQ ID NO: 2, SEQ ID NO: 3, SEQ ID NO: 5, SEQ ID NO: 9, SEQ ID NO: 25, SEQ ID NO: 26, SEQ ID NO: 27, SEQ ID NO: 28.

Wild-type beta-glucanase: The term “wild-type” beta-glucanase means a beta-glucanase expressed by a naturally occurring microorganism, such as a bacterium, archaea, yeast, or filamentous fungus found in nature.

Wash performance: The term “wash performance” is defined herein as the ability of an enzyme or a blend of enzymes to remove stains present on an object to be cleaned during e.g. wash or hard surface cleaning relative to the wash performance without one or more on the enzymes present.

### Conventions for Designation of Variants

The principles described below for beta-glucanases can be used for any protein. For purposes of the present invention, the mature polypeptide disclosed in SEQ ID NO: 26 is used to determine the corresponding amino acid residue in another beta-glucanase (e.g. a beta-glucanase variant). The amino acid sequence of another beta-glucanase is aligned with the mature polypeptide disclosed in SEQ ID NO: 26, and based on the alignment, the amino acid position number corresponding to any amino acid residue in the mature polypeptide disclosed in SEQ ID NO: 26 is determined using the Needleman-Wunsch algorithm (Needleman and Wunsch, 1970, J. Mol. Biol. 48: 443-453) as implemented in the Needle program of the EMBOSS package (EMBOSS: The European Molecular Biology Open Software Suite, Rice et al., 2000, Trends Genet. 16: 276-277), preferably version 5.0.0 or later. The parameters used are gap open penalty of 10, gap extension penalty of 0.5, and the EBLOSUM62 (EMBOSS version of BLOSUM62) substitution matrix.

Identification of the corresponding amino acid residue in another beta-glucanase can be determined by an alignment of multiple polypeptide sequences using several computer programs including, but not limited to, MUSCLE (multiple sequence comparison by log-expectation; version 3.5 or later; Edgar, 2004, Nucleic Acids Research 32: 1792-1797), MAFFT (version 6.857 or later; Katoh and Kuma, 2002, Nucleic Acids Research 30: 3059-3066; Katoh et al., 2005, Nucleic Acids Research 33: 511-518; Katoh and Toh, 2007, Bioinformatics 23: 372-374; Katoh et al., 2009, Methods in Molecular Biology 537: 39-64; Katoh and Toh, 2010, Bioinformatics 26: 1899-1900), and EMBOSS EMMA employing ClustalW (1.83 or later; Thompson et al., 1994, Nucleic Acids Research 22: 4673-4680), using their respective default parameters.

When the other enzyme has diverged from the mature polypeptide of SEQ ID NO: 26 such that traditional sequence-based comparison fails to detect their relationship (Lindahl and Elofsson, 2000, J. Mol. Biol. 295: 613-615), other pairwise sequence comparison algorithms can be used. Greater sensitivity in sequence-based searching can be attained using search programs that utilize probabilistic representations of polypeptide families (profiles) to search databases. For example, the PSI-BLAST program generates profiles through an iterative database search process and is capable of detecting remote homologs (Atschul et al., 1997, Nucleic Acids Res. 25: 3389-3402). Even greater sensitivity can be achieved if the family or superfamily for the polypeptide has one or more representatives in the protein structure databases. Programs such as GenTHREADER (Jones, 1999, J. Mol. Biol. 287: 797-815; McGuffin and Jones, 2003, Bioinformatics 19: 874-881) utilize information from a variety of sources (PSI-BLAST, secondary structure prediction, structural alignment profiles, and solvation potentials) as input to a neural network that predicts the structural fold for a query sequence. Similarly, the method of Gough et al., 2000, J. Mol. Biol. 313: 903-919, can be used to align a sequence of unknown structure with the superfamily models present in the SCOP database. These alignments can in turn be used to generate homology models for the polypeptide, and such models can be assessed for accuracy using a variety of tools developed for that purpose.

For proteins of known structure, several tools and resources are available for retrieving and generating structural alignments. For example the SCOP superfamilies of proteins have been structurally aligned, and those alignments are accessible and downloadable. 2 to 6 protein structures can be aligned using a variety of algorithms such as the distance alignment matrix (Holm and Sander, 1998, Proteins 33: 88-96) or combinatorial extension (Shindyalov and Bourne, 1998, Protein Engineering 11: 739-747), and implementation of these algorithms can additionally be utilized to query structure databases with a structure of interest in order to discover possible structural homologs (e.g., Holm and Park, 2000, Bioinformatics 16: 566-567).

In describing the variants of the present invention, the nomenclature described below is adapted for ease of reference. The accepted IUPAC single letter or three letter amino acid abbreviation is employed.

Substitutions. For an amino acid substitution, the following nomenclature is used: Original amino acid, position, substituted amino acid. Accordingly, the substitution of threonine at position 226 with alanine is designated as “Thr226Ala” or “T226A”. Multiple mutations are separated by addition marks (“+”), e.g., “Gly205Arg+Ser411Phe” or “G205R+S411F”, representing substitutions at positions 205 and 411 of glycine (G) with arginine (R) and serine (S) with phenylalanine (F), respectively.

Deletions. For an amino acid deletion, the following nomenclature is used: Original amino acid, position, *. Accordingly, the deletion of glycine at position 195 is designated as “Gly195*” or “G195*”. Multiple deletions are separated by addition marks (“+”), e.g., “Gly195*+Ser411*” or “G195*+S411*”.

Insertions. For an amino acid insertion, the following nomenclature is used: Original amino acid, position, original amino acid, inserted amino acid. Accordingly the insertion of lysine after glycine at position 195 is designated “Gly195GlyLys” or “G195GK”. An insertion of multiple amino acids is designated [Original amino acid, position, original amino acid, inserted amino acid #1, inserted amino acid #2; etc.]. For example, the insertion of lysine and alanine after glycine at position 195 is indicated as “Gly195GlyLysAla” or “G195GKA”.

In such cases the inserted amino acid residue(s) are numbered by the addition of lower case letters to the position number of the amino acid residue preceding the inserted amino acid residue(s). In the above example, the sequence would thus be:

| Parent: | Variant: |
|---|---|
| 195 | 195 195a 195b |
| G | G - K - A |

Multiple alterations. Variants comprising multiple alterations are separated by addition marks (“+”), e.g., “Arg170Tyr+Gly195Glu” or “R170Y+G195E” representing a substitution of arginine and glycine at positions 170 and 195 with tyrosine and glutamic acid, respectively.

Different alterations. Where different alterations can be introduced at a position, the different alterations are separated by a comma, e.g., “Arg170Tyr,Glu” represents a substitution of arginine at position 170 with tyrosine or glutamic acid. Thus, “Tyr167Gly,Ala+Arg170Gly,Ala” designates the following variants:

“Tyr167Gly+Arg170Gly”, “Tyr167Gly+Arg170Ala”, “Tyr167Ala+Arg170Gly”, and “Tyr167Ala+Arg170Ala”.

### DETAILED DESCRIPTION OF THE INVENTION

The present invention relates to a beta-glucanase variant, selected from the group consisting of:
a) a variant comprising one or more substitutions at the positions corresponding to positions F33 and M188 of the mature polypeptide of SEQ ID NO: 26, wherein the variant has beta-glucanase activity and wherein the variant has at least 60%, e.g., at least 61%, at least 62%, at least 63%, at least 64%, at least 65%, at least 66%, at least 67%, at least 68%, at least 69%, at least 70%, at least 71%, at least 72%, at least 73%, at least 74%, at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 95.5%, at least 96%, at least 96.5%, at least 97%, at least 97.5%, at least 98%, at least 98.5%, at least 99%, or at least 99.5%, but less than 100% sequence identity to the mature polypeptide of SEQ ID NO: 26,
b) a variant comprising one or more substitutions at the positions corresponding to positions F33 and M188 of the mature polypeptide of SEQ ID NO: 27, wherein the variant has beta-glucanase activity and wherein the variant has at least 60%, e.g., at least 61%, at least 62%, at least 63%, at least 64%, at least 65%, at least 66%, at least 67%, at least 68%, at least 69%, at least 70%, at least 71%, at least 72%, at least 73%, at least 74%, at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 95.5%, at least 96%, at least 96.5%, at least 97%, at least 97.5%, at least 98%, at least 98.5%, at least 99%, or at least 99.5%, but less than 100% sequence identity to the mature polypeptide of SEQ ID NO: 27,
c) a variant comprising one or more substitutions at the positions corresponding to positions M32 and M188 of the mature polypeptide of SEQ ID NO: 25, wherein the variant has beta-glucanase activity and wherein the variant has at least 60%, e.g., at least 61%, at least 62%, at least 63%, at least 64%, at least 65%, at least 66%, at least 67%, at least 68%, at least 69%, at least 70%, at least 71%, at least 72%, at least 73%, at least 74%, at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 95.5%, at least 96%, at least 96.5%, at least 97%, at least 97.5%, at least 98%, at least 98.5%, at least 99%, or at least 99.5%, but less than 100% sequence identity to the mature polypeptide of SEQ ID NO: 25, and
d) a variant comprising one or more substitutions at the positions corresponding to positions M29 and M180 of the mature polypeptide of SEQ ID NO: 28, wherein the variant has beta-glucanase activity and wherein the variant has at least 60%, e.g., at least 61%, at least 62%, at least 63%, at least 64%, at least 65%, at least 66%, at least 67%, at least 68%, at least 69%, at least 70%, at least 71%, at least 72%, at least 73%, at least 74%, at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 95.5%, at least 96%, at least 96.5%, at least 97%, at least 97.5%, at least 98%, at least 98.5%, at least 99%, or at least 99.5%, but less than 100% sequence identity to the mature polypeptide of SEQ ID NO: 28.

In another aspect the present invention relates to a variant of a parent beta-glucanase, the variant comprising a substitution at one or more positions corresponding to positions 33 (e.g., F33) and 188 (e.g., M188) of the mature polypeptide of SEQ ID NO: 26 using the numbering of SEQ ID NO: 26, wherein the variant has beta-glucanase activity and wherein the variant has at least 60%, e.g., at least 61%, at least 62%, at least 63%, at least 64%, at least 65%, at least 66%, at least 67%, at least 68%, at least 69%, at least 70%, at least 71%, at least 72%, at least 73%, at least 74%, at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 95.5%, at least 96%, at least 96.5%, at least 97%, at least 97.5%, at least 98%, at least 98.5%, at least 99%, or at least 99.5%, but less than 100% sequence identity to the mature polypeptide of any of: SEQ ID NO: 26, SEQ ID NO: 27, SEQ ID NO: 25, and SEQ ID NO: 28.

### Variants

The present invention provides beta-glucanase variants, comprising an alteration at one or more positions corresponding to positions 33 (e.g., F33) and 188 (e.g., M188) of the mature polypeptide of SEQ ID NO: 26 using the numbering of SEQ ID NO: 26, wherein the variant has beta-glucanase activity.

In an embodiment, the alteration is a substitution.

In an embodiment, the variant has sequence identity of at least 60%, e.g., at least 61%, at least 62%, at least 63%, at least 64%, at least 65%, at least 66%, at least 67%, at least 68%, at least 69%, at least 70%, at least 71%, at least 72%, at least 73%, at least 74%, at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 95.5%, at least 96%, at least 96.5%, at least 97%, at least 97.5%, at least 98%, at least 98.5%, at least 99%, or at least 99.5%, but less than 100%, to the amino acid sequence of the parent beta-glucanase.

In another embodiment, the variant has at least 60%, e.g., at least 61%, at least 62%, at least 63%, at least 64%, at least 65%, at least 66%, at least 67%, at least 68%, at least 69%, at least 70%, at least 71%, at least 72%, at least 73%, at least 74%, at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 95.5%, at least 96%, at least 96.5%, at least 97%, at least 97.5%, at least 98%, at least 98.5%, at least 99%, or at least 99.5%, but less than 100%, sequence identity to the mature polypeptide selected from the group consisting of: amino acids 1 to 222 of SEQ ID NO: 7 (which is also designated as SEQ ID NO: 26), amino acids 1 to 351 of SEQ ID NO: 2 (which is also designated as SEQ ID NO: 27), amino acids 1 to 351 of SEQ ID NO: 3 (which is also designated as SEQ ID NO: 27), amino acids 1 to 245 of SEQ ID NO: 5 (which is also designated as SEQ ID NO: 25), amino acids 1 to 214 of SEQ ID NO: 9 (which is also designated as SEQ ID NO: 28).

In one aspect, the number of alterations in the variants of the present invention is 1-20, e.g., 1-10 and 1-5, such as 1, 2, 3, 4, 5, 6, 7, 8, 9 or 10 alterations.

In another aspect, a variant comprises an alteration at one or more positions corresponding to positions 32 (e.g., M32), 33 (e.g., F33), 188 (e.g., M188), 29 (e.g., M29) and 180 (e.g., M180).

In another aspect, a variant comprises an alteration at two positions corresponding to any of positions 32 (e.g., M32), 33 (e.g., F33), 188 (e.g., M188), 29 (e.g., M29) and 180 (e.g., M180).

In another aspect, the variant comprises or consists of a substitution at a position corresponding to position 32 (e.g., M32). In another aspect, the amino acid at a position corresponding to position 32 (e.g., M32) is substituted with Ala, Arg, Asn, Asp, Cys, Gln, Glu, Gly, His, Ile, Leu, Lys, Phe, Pro, Ser, Thr, Trp, Tyr, or Val, preferably with Ala, Asn, Cys, Gln, Glu, Gly, Leu, Ser, Trp, Tyr, or Val, more preferred with Val, Gly, Asn, Ser or Cys. In another aspect, the variant comprises or consists of the substitution M32Y or N of the mature polypeptide of SEQ ID NO: 25.

In another aspect, the variant comprises or consists of a substitution at a position corresponding to position 188 (e.g., M188). In another aspect, the amino acid at a position corresponding to position 188 (e.g., M188) is substituted with Ala, Arg, Asn, Asp, Cys, Gln, Glu, Gly, His, Ile, Leu, Lys, Phe, Pro, Ser, Thr, Trp, Tyr, or Val, preferably with Ala, Arg, Cys, Gln, Glu, His, Leu, Phe, Pro, Ser, Thr or Tyr more preferred with Leu, His or Arg. In another aspect, the variant comprises or consists of the substitution M188H of the mature polypeptide of SEQ ID NO: 25.

In another aspect, the variant comprises or consists of a substitution at a position corresponding to position 33 (e.g., F33). In another aspect, the amino acid at a position corresponding to position 33 (e.g., F33) is substituted with Ala, Arg, Asn, Asp, Cys, Gin, Glu, Gly, His, Ile, Leu, Lys, Phe, Pro, Ser, Thr, Trp, Tyr, or Val, preferably with Ala, Asn, Cys, Gln, Glu, Gly, Leu, Ser, Trp, Tyr, or Val, more preferred with Val, Gly, Asn, Ser or Cys. In another aspect, the variant comprises or consists of the substitution F33Y or N of the mature polypeptide of SEQ ID NO: 26.

In another aspect, the variant comprises or consists of a substitution at a position corresponding to position 188 (e.g., M188). In another aspect, the amino acid at a position corresponding to position 188 (e.g., M188) is substituted with Ala, Arg, Asn, Asp, Cys, Gin, Glu, Gly, His, Ile, Leu, Lys, Phe, Pro, Ser, Thr, Trp, Tyr, or Val, preferably with Ala, Arg, Cys, Gin, Glu, His, Leu, Phe, Pro, Ser, Thr or Tyr more preferred with Leu, His or Arg. In another aspect, the variant comprises or consists of the substitution M188H of the mature polypeptide of SEQ ID NO: 26.

In another aspect, the variant comprises or consists of a substitution at a position corresponding to position 33 (e.g., F33). In another aspect, the amino acid at a position corresponding to position 33 (e.g., F33) is substituted with Ala, Arg, Asn, Asp, Cys, Gin, Glu, Gly, His, Ile, Leu, Lys, Phe, Pro, Ser, Thr, Trp, Tyr, or Val, preferably with Ala, Asn, Cys, Gin, Glu, Gly, Leu, Ser, Trp, Tyr, or Val, more preferred with Val, Gly, Asn, Ser or Cys. In another aspect, the variant comprises or consists of the substitution F33Y or N of the mature polypeptide of SEQ ID NO: 27.

In another aspect, the variant comprises or consists of a substitution at a position corresponding to position 188 (e.g., M188). In another aspect, the amino acid at a position corresponding to position 188 (e.g., M188) is substituted with Ala, Arg, Asn, Asp, Cys, Gin, Glu, Gly, His, Ile, Leu, Lys, Phe, Pro, Ser, Thr, Trp, Tyr, or Val, preferably with Ala, Arg, Cys, Gin, Glu, His, Leu, Phe, Pro, Ser, Thr or Tyr more preferred with Leu, His or Arg. In another aspect, the variant comprises or consists of the substitution M188H of the mature polypeptide of SEQ ID NO: 27.

In another aspect, the variant comprises or consists of a substitution at a position corresponding to position 29 (e.g., M29). In another aspect, the amino acid at a position corresponding to position 29 (e.g., M29) is substituted with Ala, Arg, Asn, Asp, Cys, Gln, Glu, Gly, His, Ile, Leu, Lys, Phe, Pro, Ser, Thr, Trp, Tyr, or Val, preferably with Ala, Asn, Cys, Gln, Glu, Gly, Leu, Ser, Trp, Tyr, or Val, more preferred with Val, Gly, Asn, Ser or Cys. In another aspect, the variant comprises or consists of the substitution M29Y or N of the mature polypeptide of SEQ ID NO: 28.

In another aspect, the variant comprises or consists of a substitution at a position corresponding to position 180 (e.g., M180). In another aspect, the amino acid at a position corresponding to position 180 (e.g., M180) is substituted with Ala, Arg, Asn, Asp, Cys, Gln, Glu, Gly, His, Ile, Leu, Lys, Phe, Pro, Ser, Thr, Trp, Tyr, or Val, preferably with Ala, Arg, Cys, Gln, Glu, His, Leu, Phe, Pro, Ser, Thr or Tyr more preferred with Leu, His or Arg. In another aspect, the variant comprises or consists of the substitution M180H of the mature polypeptide of SEQ ID NO: 28.

In another aspect, the variant comprises or consists of a substitution at positions corresponding to positions 32 (e.g., M32), 33 (e.g., F33), 188 (e.g., M188), 29 (e.g., M29) and 180 (e.g., M180).

In another aspect, the variant comprises or consists of an alteration at positions corresponding to positions M32V+M188L, M32V+M188H, or M32V+M188T. In another aspect, the variant comprises or consists of an alteration at positions corresponding to positions M32A+M188F such as those described above. In another aspect, the variant comprises or consists of an alteration at positions corresponding to positions M32G+M188L, M32G+M188R, M32G+M188H, or M32G+M188C. In another aspect, the variant comprises or consists of an alteration at positions corresponding to positions M32S+M188Y, M32S+M188A, or M32S+M188L. In another aspect, the variant comprises or consists of an alteration at positions corresponding to positions M32E+M188L. In another aspect, the variant comprises or consists of an alteration at positions corresponding to positions M32W+M188S. In another aspect, the variant comprises or consists of an alteration at positions corresponding to positions M32N+M188F, or M32N+M188Q. In another aspect, the variant comprises or consists of an alteration at positions corresponding to positions M32C+M188E, or M32C+M188P. In another aspect, the variant comprises or consists of an alteration at positions corresponding to positions M32Q+M188R. In another aspect, the variant comprises or consists of an alteration at positions corresponding to positions M32L+M188T. The variants may further comprise one or more additional alterations at one or more (e.g., several) other positions.

In another aspect, the variant comprises or consists of an alteration at positions corresponding to positions F33V+M188L, F33V+M188H, or F33V+M188T. In another aspect, the variant comprises or consists of an alteration at positions corresponding to positions F33A+M188F such as those described above. In another aspect, the variant comprises or consists of an alteration at positions corresponding to positions F33G+M188L, F33G+M188R, F33G+M188H, or F33G+M188C. In another aspect, the variant comprises or consists of an alteration at positions corresponding to positions F33S+M188Y, F33S+M188A, or F33S+M188L. In another aspect, the variant comprises or consists of an alteration at positions corresponding to positions F33E+M188L. In another aspect, the variant comprises or consists of an alteration at positions corresponding to positions F33W+M188S. In another aspect, the variant comprises or consists of an alteration at positions corresponding to positions F33N+M188F, or F33N+M188Q. In another aspect, the variant comprises or consists of an alteration at positions corresponding to positions F33C+M188E, or F33C+M188P. In another aspect, the variant comprises or consists of an alteration at positions corresponding to positions F33Q+M188R. In another aspect, the variant comprises or consists of an alteration at positions corresponding to positions F33L+M188T. The variants may further comprise one or more additional alterations at one or more (e.g., several) other positions.

In another aspect, the variant comprises or consists of an alteration at positions corresponding to positions M29V+M180L, M29V+M180H, or M29V+M180T, such as those described above. In another aspect, the variant comprises or consists of an alteration at positions corresponding to positions M29A+M180F such as those described above. In another aspect, the variant comprises or consists of an alteration at positions corresponding to positions M29G+M180L, M29G+M180R, M29G+M180H, or M29G+M180C. In another aspect, the variant comprises or consists of an alteration at positions corresponding to positions M29S+M180Y, M29S+M180A, or M29S+M180L. In another aspect, the variant comprises or consists of an alteration at positions corresponding to positions M29E+M180L. In another aspect, the variant comprises or consists of an alteration at positions corresponding to positions M29W+M180S. In another aspect, the variant comprises or consists of an alteration at positions corresponding to positions M29N+M180F, or M29N+M180Q. In another aspect, the variant comprises or consists of an alteration at positions corresponding to positions M29C+M180E, or M29C+M180P. In another aspect, the variant comprises or consists of an alteration at positions corresponding to positions M29Q+M180R. In another aspect, the variant comprises or consists of an alteration at positions corresponding to positions M29L+M180T. The variants may further comprise one or more additional alterations at one or more (e.g., several) other positions.

In another aspect, the variant comprises or consists of one or more (e.g., several) substitutions selected from the group consisting of: M32V+M188L; M32A+M188F; M32Y; M32V+M188H; M32G+M188L; M32N; M32G+M188R; M32S+M188Y; M32G+M188H; M32E+M188L; M188H; M32W+M188S; M32N+M188F; M32S+M188A; M32C+M188L; M32V+M188T; M32Q+M188R; M32L+M188T; M32G+M188C; M32N+M188Q; M32L+M188A; F33V+M188L; F33A+M188F; F33Y; F33V+M188H; F33G+M188L; F33N; F33G+M188R; F33S+M188Y; F33G+M188H; F33E+M188L; M188H; F33W+M188S; F33N+M188F; F33S+M188A; F33C+M188L; F33V+M188T; F33Q+M188R; F33L+M188T; F33G+M188C; F33N+M188Q; F33L+M188A; M29V+M180L; M29A+M180F; M29Y; M29V+M180H; M29G+M180L; M29N; M29G+M180R; M29S+M180Y; M29G+M180H; M29E+M180L; M180H; M29W+M180S; M29N+M180F; M29S+M180A; M29C+M180L; M29V+M180T; M29Q+M180R; M29L+M180T; M29G+M180; M29N+M180Q; and M29L+M180A.

In another aspect, the variant comprises or consists of the substitutions M32V+M188L, M32V+M188H, or M32V+M188T of the mature polypeptide selected from the group consisting of: SEQ ID NO: 25 and a polypeptide having at least 65%, at least 70%, at least 75%, at least 80%, at least 85%, at least 90%, at least 91%, at least 92%, at least 93%, at least 94%, at least 95%, at least 96%, at least 97%, at least 98%, at least 99% identity to the mature polypeptide of SEQ ID NO: 25 which has beta-glucanase activity. In another aspect, the variant comprises or consists of the substitutions M32A+M188F of the mature polypeptide selected from the group consisting of: SEQ ID NO: 25 and polypeptide having at least 65%, at least 70%, at least 75%, at least 80%, at least 85%, at least 90%, at least 91%, at least 92%, at least 93%, at least 94%, at least 95%, at least 96%, at least 97%, at least 98%, at least 99% identity to the mature polypeptide of SEQ ID NO: 25 which has beta-glucanase activity. In another aspect, the variant comprises or consists of the substitutions M32G+M188L, M32G+M188R, M32G+M188H, or M32G +M188C of the mature polypeptide selected from the group consisting of: SEQ ID NO: 25 and polypeptide having at least 65%, at least 70%, at least 75%, at least 80%, at least 85%, at least 90%, at least 91%, at least 92%, at least 93%, at least 94%, at least 95%, at least 96%, at least 97%, at least 98%, at least 99% identity to the mature polypeptide of SEQ ID NO: 25 which has beta-glucanase activity. In another aspect, the variant comprises or consists of the substitutions M32S+M188Y, M32S+M188A, or M32S+M188L of the mature polypeptide selected from the group consisting of: SEQ ID NO: 25 and a polypeptide having at least 65%, at least 70%, at least 75%, at least 80%, at least 85%, at least 90%, at least 91%, at least 92%, at least 93%, at least 94%, at least 95%, at least 96%, at least 97%, at least 98%, at least 99% identity to the mature polypeptide of SEQ ID NO: 25 which has beta-glucanase activity. In another aspect, the variant comprises or consists of the substitutions M32E+M188L of the mature polypeptide selected from the group consisting of: SEQ ID NO: 25 and a polypeptide having at least 65%, at least 70%, at least 75%, at least 80%, at least 85%, at least 90%, at least 91%, at least 92%, at least 93%, at least 94%, at least 95%, at least 96%, at least 97%, at least 98%, at least 99% identity to the mature polypeptide of SEQ ID NO: 25 which has beta-glucanase activity. In another aspect, the variant comprises or consists of the substitutions M32W+M188S of the mature polypeptide selected from the group consisting of: SEQ ID NO: 25 and a polypeptide having at least 65%, at least 70%, at least 75%, at least 80%, at least 85%, at least 90%, at least 91%, at least 92%, at least 93%, at least 94%, at least 95%, at least 96%, at least 97%, at least 98%, at least 99% identity to the mature polypeptide of SEQ ID NO: 25 which has beta-glucanase activity. In another aspect, the variant comprises or consists of the substitutions M32N+M188F, or M32N+M188Q of the mature polypeptide selected from the group consisting of: SEQ ID NO: 25 and polypeptide having at least 65%, at least 70%, at least 75%, at least 80%, at least 85%, at least 90%, at least 91%, at least 92%, at least 93%, at least 94%, at least 95%, at least 96%, at least 97%, at least 98%, at least 99% identity to the mature polypeptide of SEQ ID NO: 25 which has beta-glucanase activity. In another aspect, the variant comprises or consists of the substitutions M32C+M188E, or M32C+M188P of the mature polypeptide selected from the group consisting of: SEQ ID NO: 25 and polypeptide having at least 65%, at least 70%, at least 75%, at least 80%, at least 85%, at least 90%, at least 91%, at least 92%, at least 93%, at least 94%, at least 95%, at least 96%, at least 97%, at least 98%, at least 99% identity to the mature polypeptide of SEQ ID NO: 25 which has beta-glucanase activity. In another aspect, the variant comprises or consists of the substitutions M32Q+M188R of the mature polypeptide selected from the group consisting of: SEQ ID NO: 25 and polypeptide having at least 65%, at least 70%, at least 75%, at least 80%, at least 85%, at least 90%, at least 91%, at least 92%, at least 93%, at least 94%, at least 95%, at least 96%, at least 97%, at least 98%, at least 99% identity to the mature polypeptide of SEQ ID NO: 25 which has beta-glucanase activity. In another aspect, the variant comprises or consists of the substitutions M32L+M188T of the mature polypeptide selected from the group consisting of: SEQ ID NO: 25 and polypeptide having at least 65%, at least 70%, at least 75%, at least 80%, at least 85%, at least 90%, at least 91%, at least 92%, at least 93%, at least 94%, at least 95%, at least 96%, at least 97%, at least 98%, at least 99% identity to the mature polypeptide of SEQ ID NO: 25 which has beta-glucanase activity. The variants may further comprise one or more additional alterations at one or more (e.g., several) other positions.

In another aspect, the variant comprises or consists of the substitutions F33V+M188L, F33V+M188H, or F33V+M188T of the mature polypeptide selected from the group consisting of: SEQ ID NO: 26 and SEQ ID NO: 27 and polypeptide having at least 65%, at least 70%, at least 75%, at least 80%, at least 85%, at least 90%, at least 91%, at least 92%, at least 93%, at least 94%, at least 95%, at least 96%, at least 97%, at least 98%, at least 99% identity to the mature polypeptide of SEQ ID NO: 26 or SEQ ID NO: 27 which has beta-glucanase activity. In another aspect, the variant comprises or consists of the substitutions F33A+M188F of the mature polypeptide selected from the group consisting of: SEQ ID NO: 26 and SEQ ID NO: 27 and polypeptide having at least 65%, at least 70%, at least 75%, at least 80%, at least 85%, at least 90%, at least 91%, at least 92%, at least 93%, at least 94%, at least 95%, at least 96%, at least 97%, at least 98%, at least 99% identity to the mature polypeptide of SEQ ID NO: 26 or SEQ ID NO: 27 which has beta-glucanase activity. In another aspect, the variant comprises or consists of the substitutions F33G+M188L, F33G+M188R, F33G+M188H, or F33G+M188C of the mature polypeptide selected from the group consisting of: SEQ ID NO: 26 and SEQ ID NO: 27 and polypeptide having at least 65%, at least 70%, at least 75%, at least 80%, at least 85%, at least 90%, at least 91%, at least 92%, at least 93%, at least 94%, at least 95%, at least 96%, at least 97%, at least 98%, at least 99% identity to the mature polypeptide of SEQ ID NO: 26 or SEQ ID NO: 27 which has beta-glucanase activity. In another aspect, the variant comprises or consists of the substitutions F33S+M188Y, F33S+M188A, or F33S+M188L of the mature polypeptide selected from the group consisting of: SEQ ID NO: 26 and SEQ ID NO: 27 and polypeptide having at least 65%, at least 70%, at least 75%, at least 80%, at least 85%, at least 90%, at least 91%, at least 92%, at least 93%, at least 94%, at least 95%, at least 96%, at least 97%, at least 98%, at least 99% identity to the mature polypeptide of SEQ ID NO: 26 or SEQ ID NO: 27 which has beta-glucanase activity. In another aspect, the variant comprises or consists of the substitutions F33E+M188L of the mature polypeptide selected from the group consisting of: SEQ ID NO: 26 and SEQ ID NO: 27 and polypeptide having at least 65%, at least 70%, at least 75%, at least 80%, at least 85%, at least 90%, at least 91%, at least 92%, at least 93%, at least 94%, at least 95%, at least 96%, at least 97%, at least 98%, at least 99% identity to the mature polypeptide of SEQ ID NO: 26 or SEQ ID NO: 27 which has beta-glucanase activity. In another aspect, the variant comprises or consists of the substitutions F33W+M188S of the mature polypeptide selected from the group consisting of: SEQ ID NO: 26 and SEQ ID NO: 27 and polypeptide having at least 65%, at least 70%, at least 75%, at least 80%, at least 85%, at least 90%, at least 91%, at least 92%, at least 93%, at least 94%, at least 95%, at least 96%, at least 97%, at least 98%, at least 99% identity to the mature polypeptide of SEQ ID NO: 26 or SEQ ID NO: 27 which has beta-glucanase activity. In another aspect, the variant comprises or consists of the substitutions F33N+M188F, or F33N+M188Q of the mature polypeptide selected from the group consisting of: SEQ ID NO: 26 and SEQ ID NO: 27 and polypeptide having at least 65%, at least 70%, at least 75%, at least 80%, at least 85%, at least 90%, at least 91%, at least 92%, at least 93%, at least 94%, at least 95%, at least 96%, at least 97%, at least 98%, at least 99% identity to the mature polypeptide of SEQ ID NO: 26 or SEQ ID NO: 27 which has beta-glucanase activity. In another aspect, the variant comprises or consists of the substitutions F33C+M188E, or F33C+M188P of the mature polypeptide selected from the group consisting of: SEQ ID NO: 26 and SEQ ID NO: 27 and polypeptide having at least 65%, at least 70%, at least 75%, at least 80%, at least 85%, at least 90%, at least 91%, at least 92%, at least 93%, at least 94%, at least 95%, at least 96%, at least 97%, at least 98%, at least 99% identity to the mature polypeptide of SEQ ID NO: 26 or SEQ ID NO: 27 which has beta-glucanase activity. In another aspect, the variant comprises or consists of the substitutions F33Q+M188R of the mature polypeptide selected from the group consisting of: SEQ ID NO: 26 and SEQ ID NO: 27 and polypeptide having at least 65%, at least 70%, at least 75%, at least 80%, at least 85%, at least 90%, at least 91%, at least 92%, at least 93%, at least 94%, at least 95%, at least 96%, at least 97%, at least 98%, at least 99% identity to the mature polypeptide of SEQ ID NO: 26 or SEQ ID NO: 27 which has beta-glucanase activity. In another aspect, the variant comprises or consists of the substitutions F33L+M188T of the mature polypeptide selected from the group consisting of: SEQ ID NO: 26 and SEQ ID NO: 27 and polypeptide having at least 65%, at least 70%, at least 75%, at least 80%, at least 85%, at least 90%, at least 91%, at least 92%, at least 93%, at least 94%, at least 95%, at least 96%, at least 97%, at least 98%, at least 99% identity to the mature polypeptide of SEQ ID NO: 26 or SEQ ID NO: 27 which has beta-glucanase activity. The variants may further comprise one or more additional alterations at one or more (e.g., several) other positions.

In another aspect, the variant comprises or consists of the substitutions M29V+M180L, M29V+M180H, or M29V+M180T of the mature polypeptide selected from the group consisting of: SEQ ID NO: 28 and polypeptide having at least 65%, at least 70%, at least 75%, at least 80%, at least 85%, at least 90%, at least 91%, at least 92%, at least 93%, at least 94%, at least 95%, at least 96%, at least 97%, at least 98%, at least 99% identity to the mature polypeptide of SEQ ID NO: 28 which has beta-glucanase activity. In another aspect, the variant comprises or consists of the substitutions M29A+M180F of the mature polypeptide selected from the group consisting of: SEQ ID NO: 28 and polypeptide having at least 65%, at least 70%, at least 75%, at least 80%, at least 85%, at least 90%, at least 91%, at least 92%, at least 93%, at least 94%, at least 95%, at least 96%, at least 97%, at least 98%, at least 99% identity to the mature polypeptide of SEQ ID NO: 28 which has beta-glucanase activity. In another aspect, the variant comprises or consists of the substitutions M29G+M180L, M29G+M180R, M29G+M180H, or M29G+M180C of the mature polypeptide selected from the group consisting of: SEQ ID NO: 28 and polypeptide having at least 65%, at least 70%, at least 75%, at least 80%, at least 85%, at least 90%, at least 91%, at least 92%, at least 93%, at least 94%, at least 95%, at least 96%, at least 97%, at least 98%, at least 99% identity to the mature polypeptide of SEQ ID NO: 28 which has beta-glucanase activity. In another aspect, the variant comprises or consists of the substitutions M29S+M180Y, M29S+M180A, or M29S+M180L of the mature polypeptide selected from the group consisting of: SEQ ID NO: 28 and polypeptide having at least 65%, at least 70%, at least 75%, at least 80%, at least 85%, at least 90%, at least 91%, at least 92%, at least 93%, at least 94%, at least 95%, at least 96%, at least 97%, at least 98%, at least 99% identity to the mature polypeptide of SEQ ID NO: 28 which has beta-glucanase activity. In another aspect, the variant comprises or consists of the substitutions M29E+M180L of the mature polypeptide selected from the group consisting of: SEQ ID NO: 28 and polypeptide having at least 65%, at least 70%, at least 75%, at least 80%, at least 85%, at least 90%, at least 91%, at least 92%, at least 93%, at least 94%, at least 95%, at least 96%, at least 97%, at least 98%, at least 99% identity to the mature polypeptide of SEQ ID NO: 28 which has beta-glucanase activity. In another aspect, the variant comprises or consists of the substitutions M29W+M180S of the mature polypeptide selected from the group consisting of: SEQ ID NO: 28 and polypeptide having at least 65%, at least 70%, at least 75%, at least 80%, at least 85%, at least 90%, at least 91%, at least 92%, at least 93%, at least 94%, at least 95%, at least 96%, at least 97%, at least 98%, at least 99% identity to the mature polypeptide of SEQ ID NO: 28 which has beta-glucanase activity. In another aspect, the variant comprises or consists of the substitutions M29N+M180F, or M29N+M180Q of the mature polypeptide selected from the group consisting of: SEQ ID NO: 28 and polypeptide having at least 65%, at least 70%, at least 75%, at least 80%, at least 85%, at least 90%, at least 91%, at least 92%, at least 93%, at least 94%, at least 95%, at least 96%, at least 97%, at least 98%, at least 99% identity to the mature polypeptide of SEQ ID NO: 28 which has beta-glucanase activity. In another aspect, the variant comprises or consists of the substitutions M29C+M180E, P of the mature polypeptide selected from the group consisting of: SEQ ID NO: 28 and polypeptide having at least 65%, at least 70%, at least 75%, at least 80%, at least 85%, at least 90%, at least 91%, at least 92%, at least 93%, at least 94%, at least 95%, at least 96%, at least 97%, at least 98%, at least 99% identity to the mature polypeptide of SEQ ID NO: 28 which has beta-glucanase activity. In another aspect, the variant comprises or consists of the substitutions M29Q+M180R of the mature polypeptide selected from the group consisting of: SEQ ID NO: 28 and polypeptide having at least 65%, at least 70%, at least 75%, at least 80%, at least 85%, at least 90%, at least 91%, at least 92%, at least 93%, at least 94%, at least 95%, at least 96%, at least 97%, at least 98%, at least 99% identity to the mature polypeptide of SEQ ID NO: 28 which has beta-glucanase activity. In another aspect, the variant comprises or consists of the substitutions M29L+M180T of the mature polypeptide selected from the group consisting of: SEQ ID NO: 28 and polypeptide having at least 65%, at least 70%, at least 75%, at least 80%, at least 85%, at least 90%, at least 91%, at least 92%, at least 93%, at least 94%, at least 95%, at least 96%, at least 97%, at least 98%, at least 99% identity to the mature polypeptide of SEQ ID NO: 28 which has beta-glucanase activity. The variants of the invention may further comprise one or more additional alterations at one or more (e.g., several) other positions.

The amino acid changes may be of a minor nature, that is conservative amino acid substitutions or insertions that do not significantly affect the folding and/or activity of the protein; small deletions, typically of 1-30 amino acids; small amino- or carboxyl-terminal extensions, such as an amino-terminal methionine residue; a small linker peptide of up to 20-25 residues; or a small extension that facilitates purification by changing net charge or another function, such as a poly-histidine tract, an antigenic epitope or a binding domain.

Examples of conservative substitutions are within the groups of basic amino acids (arginine, lysine and histidine), acidic amino acids (glutamic acid and aspartic acid), polar amino acids (glutamine and asparagine), hydrophobic amino acids (leucine, isoleucine and valine), aromatic amino acids (phenylalanine, tryptophan and tyrosine), and small amino acids (glycine, alanine, serine, threonine and methionine). Amino acid substitutions that do not generally alter specific activity are known in the art and are described, for example, by H. Neurath and R. L. Hill, 1979, In, The Proteins, Academic Press, New York. Common substitutions are Ala/Ser, Val/Ile, Asp/Glu, Thr/Ser, Ala/Gly, Ala/Thr, Ser/Asn, Ala/Val, Ser/Gly, Tyr/Phe, Ala/Pro, Lys/Arg, Asp/Asn, Leu/Ile, Leu/Val, Ala/Glu, and Asp/Gly.

Alternatively, the amino acid changes are of such a nature that the physico-chemical properties of the polypeptides are altered. For example, amino acid changes may improve the thermal stability of the polypeptide, alter the substrate specificity, change the pH optimum, and the like.

Essential amino acids in a polypeptide can be identified according to procedures known in the art, such as site-directed mutagenesis or alanine-scanning mutagenesis (Cunningham and Wells, 1989, Science 244: 1081-1085). In the latter technique, single alanine mutations are introduced at every residue in the molecule, and the resultant mutant molecules are tested for beta-glucanase activity to identify amino acid residues that are critical to the activity of the molecule. See also, Hilton et al., 1996, J. Biol. Chem. 271: 4699-4708. The active site of the enzyme or other biological interaction can also be determined by physical analysis of structure, as determined by such techniques as nuclear magnetic resonance, crystallography, electron diffraction, or photoaffinity labeling, in conjunction with mutation of putative contact site amino acids. See, for example, de Vos et al., 1992, Science 255: 306-312; Smith et al., 1992, J. Mol. Biol. 224: 899-904; Wlodaver et al., 1992, FEBS Lett. 309: 59-64. The identity of essential amino acids can also be inferred from an alignment with a related polypeptide.

In an embodiment, the variant has improved oxidation stability compared to the parent enzyme.

In an embodiment, the variant has improved thermostability compared to the parent enzyme.

In one embodiment beta-glucanase activity of the variant of the present invention is not an endo-cellulase activity on β-1,4 linkages between D-glucose units of cellulose. In another embodiment beta-glucanase activity of the variant of the present invention comprises licheninase EC 3.2.1.73 activity. In a further embodiment beta-glucanase activity of the variant of the present invention is licheninase EC 3.2.1.73 activity.

In one embodiment the variant of the present invention is capable of having beta-glucanase activity in an aqueous solution with a pH selected in the range from about 7.5 to about 13.5, wherein said aqueous solution optionally comprises a bleaching agent, preferably said pH is selected in the range from about 7.5 to about 12.5, further preferably said pH is selected in the range from about 8.5 to about 11.5, most preferably said pH is selected in the range from about 9.5 to about 10.5.

In another embodiment the variant is capable of having beta-glucanase activity in an aqueous solution at a temperature selected in the range from about 20° C. to about 75° C., wherein said aqueous solution optionally comprises a bleaching agent, preferably said temperature is selected in the range from about 40° C. to about 60° C.

In another embodiment the variant is capable of having beta-glucanase activity for at least 15 minutes, preferably for at least 30 minutes, further preferably for at least 60 minutes, further most preferably for at least 90 minutes, further most preferably for at least 120 minutes.

### Parent Beta-Glucanases

The parent beta-glucanase may be a Bacillus beta-glucanase having a methionine or phenylalanine residue in at least one position corresponding to positions: 32 (e.g., M32) and 188 (e.g., M188), e.g., as in the polypeptide of SEQ ID NO: 25, 33 (e.g., F33) and 188 (e.g., M188), e.g., as in the polypeptide of SEQ ID NO: 26, 33 (e.g., F33) and M188 (e.g., M188), e.g., as in the polypeptide of SEQ ID NO: 27 and 29 (e.g., M29) and 180 (e.g., M180), e.g., as in the polypeptide of SEQ ID NO: 28,

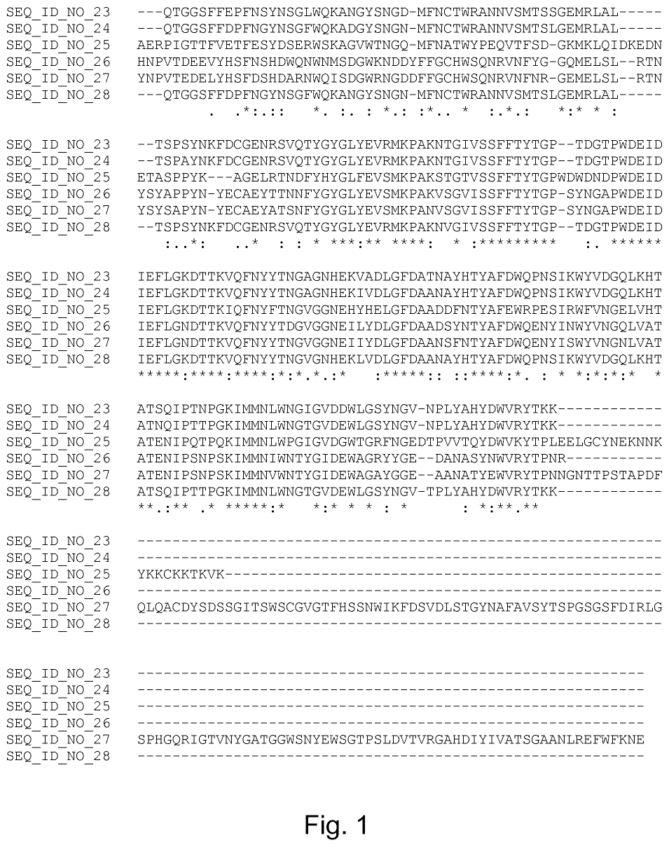

The two methionines or a combination of phenylalanine and methionine are conserved among Bacillus beta-glucanases and also to some extent among alkaline Bacillus beta-glucanases having low sequence identity to the mature polypeptide selected from the group consisting of: SEQ ID NO: 25, SEQ ID NO: 26, SEQ ID NO: 27 and SEQ ID NO: 28, e.g., down to around 50% identical or even lower sequence identity (e.g., FIG. 1).

Preferred parent beta-glucanases according to the invention include: SEQ ID NO: 25, SEQ ID NO: 26, SEQ ID NO: 27, SEQ ID NO: 28, SEQ ID NO: 2, SEQ ID NO: 3, SEQ ID NO: 5, SEQ ID NO: 7 and SEQ ID NO: 9. Other suitable parent beta-glucanases according to the invention include beta-glucanases with SEQ ID NO: 23 and SEQ ID NO: 24.

FIG. 1 shows a multiple sequence alignment of mature beta-glucanases with SEQ ID NO: 23, SEQ ID NO: 24, SEQ ID NO: 25, SEQ ID NO: 26, SEQ ID NO: 27 and SEQ ID NO: 28 and clearly demonstrates that the aligned sequences are homologous and contain methionine or phenylalanine residues in positions corresponding to positions selected from a group consisting of: M32 and M188 of the polypeptide of SEQ ID NO: 25, F33 and M188 of the polypeptide of SEQ ID NO: 26, F33 and M188 of the polypeptide of SEQ ID NO: 27 and M29 and M180 of the polypeptide of SEQ ID NO: 28.

The parent beta-glucanase may be (a) a polypeptide having at least 60%, e.g., at least 61%, at least 62%, at least 63%, at least 64%, at least 65%, at least 66%, at least 67%, at least 68%, at least 69%, at least 70%, at least 71%, at least 72%, at least 73%, at least 74%, at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 95.5%, at least 96%, at least 96.5%, at least 97%, at least 97.5%, at least 98%, at least 98.5%, at least 99%, or at least 99.5%, sequence identity to the polypeptide selected from the group consisting of: SEQ ID NO: 2, SEQ ID NO: 3, SEQ ID NO: 5, SEQ ID NO: 7, SEQ ID NO: 9, SEQ ID NO: 25, SEQ ID NO: 26, SEQ ID NO: 27 and SEQ ID NO: 28; or (b) a polypeptide encoded by a polynucleotide that hybridizes under low stringency conditions, preferably medium stringency conditions, further preferably medium-high stringency conditions, further most preferably high stringency conditions, further most preferably very high stringency conditions, with (i) the mature polypeptide coding sequence of the sequence selected from the group consisting of: SEQ ID NO: 6, SEQ ID NO: 1, SEQ ID NO: 4, SEQ ID NO: 8, or (ii) the full-length complement of (i) or (ii).

In another aspect, the parent has a sequence identity to the mature polypeptide selected from the group consisting of: SEQ ID NO: 25, SEQ ID NO: 26, SEQ ID NO: 27 and SEQ ID NO: 28, of at least 60%, e.g., e.g., at least 61%, at least 62%, at least 63%, at least 64%, at least 65%, at least 66%, at least 67%, at least 68%, at least 69%, at least 70%, at least 71%, at least 72%, at least 73%, at least 74%, at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98% or at least 99%, or 100%, which have beta-glucanase activity. In one aspect, the amino acid sequence of the parent differs by up to 10 amino acids, e.g., 1, 2, 3, 4, 5, 6, 7, 8, 9, or 10, from the mature polypeptide selected from the group consisting of: SEQ ID NO: 25, SEQ ID NO: 26, SEQ ID NO: 27 and SEQ ID NO: 28.

In another aspect, the parent comprises or consists of the amino acid sequence selected from the group consisting of: SEQ ID NO: 25, SEQ ID NO: 26, SEQ ID NO: 27 and SEQ ID NO: 28. In another aspect, the parent comprises or consists of the polypeptide selected from the group consisting of: SEQ ID NO: 2, SEQ ID NO: 3, SEQ ID NO: 5, SEQ ID NO: 7 and SEQ ID NO: 9, SEQ ID NO: 23, SEQ ID NO: 24, SEQ ID NO: 25, SEQ ID NO: 26, SEQ ID NO: 27 and SEQ ID NO: 28.

In another aspect, the parent is a fragment of the polypeptide selected from the group consisting of: SEQ ID NO: 2, SEQ ID NO: 3, SEQ ID NO: 5, SEQ ID NO: 7 and SEQ ID NO: 9, SEQ ID NO: 25, SEQ ID NO: 26, SEQ ID NO: 27 and SEQ ID NO: 28 containing at least 180 amino acid residues, e.g., at least 200 and at least 210 amino acid residues.

In another embodiment, the parent is an allelic variant of the polypeptide selected from the group consisting of: SEQ ID NO: 2, SEQ ID NO: 3, SEQ ID NO: 5, SEQ ID NO: 7 and SEQ ID NO: 9, SEQ ID NO: 25, SEQ ID NO: 26, SEQ ID NO: 27 and SEQ ID NO: 28.

The polynucleotide selected from the group consisting of: SEQ ID NO: 1, SEQ ID NO: 4, SEQ ID NO: 6, SEQ ID NO: 8 or a subsequence thereof, as well as the polypeptide selected from the group consisting of: SEQ ID NO: 2, SEQ ID NO: 3, SEQ ID NO: 5, SEQ ID NO: 7 and SEQ ID NO: 9, SEQ ID NO: 25, SEQ ID NO: 26, SEQ ID NO: 27 and SEQ ID NO: 28 or a fragment thereof, may be used to design nucleic acid probes to identify and clone DNA encoding a parent from strains of different genera or species according to methods well known in the art. In particular, such probes can be used for hybridization with the genomic DNA or cDNA of a cell of interest, following standard Southern blotting procedures, in order to identify and isolate the corresponding gene therein. Such probes can be considerably shorter than the entire sequence, but should be at least 15, e.g., at least 25, at least 35, or at least 70 nucleotides in length. Preferably, the nucleic acid probe is at least 100 nucleotides in length, e.g., at least 200 nucleotides, at least 300 nucleotides, at least 400 nucleotides, at least 500 nucleotides, at least 600 nucleotides, at least 700 nucleotides, at least 800 nucleotides, or at least 900 nucleotides in length. Both DNA and RNA probes can be used. The probes are typically labeled for detecting the corresponding gene (for example, with ³²P, ³H, ³⁵S, biotin, or avidin). Such probes are encompassed by the present invention.

A genomic DNA or cDNA library prepared from such other strains may be screened for DNA that hybridizes with the probes described above and encodes a parent. Genomic or other DNA from such other strains may be separated by agarose or polyacrylamide gel electrophoresis, or other separation techniques. DNA from the libraries or the separated DNA may be transferred to and immobilized on nitrocellulose or other suitable carrier material. In order to identify a clone or DNA that hybridizes with the nucleotide sequence encoding a polynucleotide selected from the group consisting of: SEQ ID NO: 2, SEQ ID NO: 3, SEQ ID NO: 5, SEQ ID NO: 7 and SEQ ID NO: 9, SEQ ID NO: 25, SEQ ID NO: 26, SEQ ID NO: 27 and SEQ ID NO: 28 or a subsequence thereof, the carrier material is used in a Southern blot.

For purposes of the present invention, hybridization indicates that the polynucleotide hybridizes to a labeled nucleic acid probe corresponding to (i) the polypeptide coding sequence selected from the group consisting of: SEQ ID NO: 2, SEQ ID NO: 3, SEQ ID NO: 5, SEQ ID NO: 7 and SEQ ID NO: 9, SEQ ID NO: 25, SEQ ID NO: 26, SEQ ID NO: 27 and SEQ ID NO: 28; (ii) the full-length complement thereof; or (iii) a subsequence thereof; under very low to very high stringency conditions. Molecules to which the nucleic acid probe hybridizes under these conditions can be detected using, for example, X-ray film or any other detection means known in the art.

In one aspect, the nucleic acid probe is a polynucleotide that encodes the polypeptide selected from the group consisting of: SEQ ID NO: 2, SEQ ID NO: 3, SEQ ID NO: 5, SEQ ID NO: 7 and SEQ ID NO: 9, SEQ ID NO: 25, SEQ ID NO: 26, SEQ ID NO: 27 and SEQ ID NO: 28; or a fragment thereof.

The polypeptide may be a hybrid polypeptide in which a region of one polypeptide is fused at the N-terminus or the C-terminus of a region of another polypeptide.

The parent may be a fusion polypeptide or cleavable fusion polypeptide in which another polypeptide is fused at the N-terminus or the C-terminus of the polypeptide of the present invention. A fusion polypeptide is produced by fusing a polynucleotide encoding another polypeptide to a polynucleotide of the present invention. Techniques for producing fusion polypeptides are known in the art, and include ligating the coding sequences encoding the polypeptides so that they are in frame and that expression of the fusion polypeptide is under control of the same promoter(s) and terminator. Fusion polypeptides may also be constructed using intein technology in which fusion polypeptides are created post-translationally (Cooper et al., 1993, EMBO J. 12: 2575-2583; Dawson et al., 1994, Science 266: 776-779).

A fusion polypeptide can further comprise a cleavage site between the two polypeptides. Upon secretion of the fusion protein, the site is cleaved releasing the two polypeptides. Examples of cleavage sites include, but are not limited to, the sites disclosed in Martin et al., 2003, J. Ind. Microbiol. Biotechnol. 3: 568-576; Svetina et al., 2000, J. Biotechnol. 76: 245-251; Rasmussen-Wilson et al., 1997, Appl. Environ. Microbiol. 63: 3488-3493; Ward et al., 1995, Biotechnology 13: 498-503; and Contreras et al., 1991, Biotechnology 9: 378-381; Eaton et al., 1986, Biochemistry 25: 505-512; Collins-Racie et al., 1995, Biotechnology 13: 982-987; Carter et al., 1989, Proteins: Structure, Function, and Genetics 6: 240-248; and Stevens, 2003, Drug Discovery World 4: 35-48.

The parent may be obtained from microorganisms of any genus. For purposes of the present invention, the term “obtained from” as used herein in connection with a given source shall mean that the parent encoded by a polynucleotide is produced by the source or by a strain in which the polynucleotide from the source has been inserted. In one aspect, the parent is secreted extracellularly.

The parent may be a bacterial beta-glucanase. For example, the parent may be a Gram-positive bacterial polypeptide such as a Bacillus, Clostridium, Enterococcus, Geobacillus, Lactobacillus, Lactococcus, Oceanobacillus, Staphylococcus, Streptococcus, or Streptomyces beta-glucanase, or a Gram-negative bacterial polypeptide such as a Campylobacter, E. coli, Flavobacterium, Fusobacterium, Helicobacter, Ilyobacter, Neisseria, Pseudomonas, Salmonella, or Ureaplasma beta-glucanase.

In one aspect, the parent is a Bacillus alkalophilus, Bacillus amyloliquefaciens, Bacillus brevis, Bacillus circulans, Bacillus clausii, Bacillus coagulans, Bacillus firmus, Bacillus lautus, Bacillus lentus, Bacillus licheniformis, Bacillus megaterium, Bacillus pumilus, Bacillus stearothermophilus, Bacillus subtilis, or Bacillus thuringiensis beta-glucanase.

In another aspect, the parent is a Streptococcus equisimilis, Streptococcus pyogenes, Streptococcus uberis, or Streptococcus equi subsp. Zooepidemicus beta-glucanase.

In another aspect, the parent is a Streptomyces achromogenes, Streptomyces avermitilis, Streptomyces coelicolor, Streptomyces griseus, or Streptomyces lividans beta-glucanase.

It will be understood that for the aforementioned species, the invention encompasses both the perfect and imperfect states, and other taxonomic equivalents, e.g., anamorphs, regardless of the species name by which they are known. Those skilled in the art will readily recognize the identity of appropriate equivalents.

Strains of these species are readily accessible to the public in a number of culture collections, such as the American Type Culture Collection (ATCC), Deutsche Sammlung von Mikroorganismen and Zellkulturen GmbH (DSMZ), Centraalbureau Voor Schimmelcultures (CBS), and Agricultural Research Service Patent Culture Collection, Northern Regional Research Center (NRRL).

The parent may be identified and obtained from other sources including microorganisms isolated from nature (e.g., soil, composts, water, etc.) or DNA samples obtained directly from natural materials (e.g., soil, composts, water, etc.) using the above-mentioned probes. Techniques for isolating microorganisms and DNA directly from natural habitats are well known in the art. A polynucleotide encoding a parent may then be obtained by similarly screening a genomic DNA or cDNA library of another microorganism or mixed DNA sample. Once a polynucleotide encoding a parent has been detected with the probe(s), the polynucleotide can be isolated or cloned by utilizing techniques that are known to those of ordinary skill in the art (see, e.g., Sambrook et al., 1989, supra).

### Preparation of Variants

The present invention also relates to methods for obtaining a variant having beta-glucanase activity, comprising: (a) introducing into a parent beta-glucanase a substitution at one or more positions corresponding to positions 33 (e.g., F33) and 188 (e.g., M188) of the mature polypeptide of SEQ ID NO: 26 using the numbering of SEQ ID NO: 26, wherein the variant has beta-glucanase activity; and recovering the variant.

The present invention also relates to methods for obtaining a variant having beta-glucanase activity, comprising: (a) introducing into a parent beta-glucanase a substitution at one or more positions corresponding to positions 33 (e.g., F33) and 188 (e.g., M188) of the mature polypeptide of SEQ ID NO: 27 using the numbering of SEQ ID NO: 27, wherein the variant has beta-glucanase activity; and recovering the variant.

The present invention also relates to methods for obtaining a variant having beta-glucanase activity, comprising: (a) introducing into a parent beta-glucanase a substitution at one or more positions corresponding to positions 32 (e.g., M32) and 188 (e.g., M188) of the mature polypeptide of SEQ ID NO: 25 using the numbering of SEQ ID NO: 25, wherein the variant has beta-glucanase activity; and recovering the variant.

The present invention also relates to methods for obtaining a variant having beta-glucanase activity, comprising: (a) introducing into a parent beta-glucanase a substitution at one or more positions corresponding to positions 29 (e.g., M29) and 180 (e.g., M180) of the mature polypeptide of SEQ ID NO: 28 using the numbering of SEQ ID NO: 28, wherein the variant has beta-glucanase activity; and recovering the variant.

The variants can be prepared using any mutagenesis procedure known in the art, such as site-directed mutagenesis, synthetic gene construction, semi-synthetic gene construction, random mutagenesis, shuffling, etc.

Site-directed mutagenesis is a technique in which one or more (e.g., several) mutations are introduced at one or more defined sites in a polynucleotide encoding the parent.

Site-directed mutagenesis can be accomplished in vitro by PCR involving the use of oligonucleotide primers containing the desired mutation. Site-directed mutagenesis can also be performed in vitro by cassette mutagenesis involving the cleavage by a restriction enzyme at a site in the plasmid comprising a polynucleotide encoding the parent and subsequent ligation of an oligonucleotide containing the mutation in the polynucleotide. Usually the restriction enzyme that digests the plasmid and the oligonucleotide is the same, permitting sticky ends of the plasmid and the insert to ligate to one another. See, e.g., Scherer and Davis, 1979, Proc. Natl. Acad. Sci. USA 76: 4949-4955; and Barton et al., 1990, Nucleic Acids Res. 18: 7349-4966.

Site-directed mutagenesis can also be accomplished in vivo by methods known in the art. See, e.g., U.S. Patent Application Publication No. 2004/0171154; Storici et al., 2001, Nature Biotechnol. 19: 773-776; Kren et al., 1998, Nat. Med. 4: 285-290; and Calissano and Macino, 1996, Fungal Genet. Newslett. 43: 15-16.

Any site-directed mutagenesis procedure can be used in the present invention. There are many commercial kits available that can be used to prepare variants.

Synthetic gene construction entails in vitro synthesis of a designed polynucleotide molecule to encode a polypeptide of interest. Gene synthesis can be performed utilizing a number of techniques, such as the multiplex microchip-based technology described by Tian et al. (2004, Nature 432: 1050-1054) and similar technologies wherein oligonucleotides are synthesized and assembled upon photo-programmable microfluidic chips.

Single or multiple amino acid substitutions, deletions, and/or insertions can be made and tested using known methods of mutagenesis, recombination, and/or shuffling, followed by a relevant screening procedure, such as those disclosed by Reidhaar-Olson and Sauer, 1988, Science 241: 53-57; Bowie and Sauer, 1989, Proc. Natl. Acad. Sci. USA 86: 2152-2156; WO 95/17413; or WO 95/22625. Other methods that can be used include error-prone PCR, phage display (e.g., Lowman et al., 1991, Biochemistry 30: 10832-10837; U.S. Pat. No. 5,223,409; WO 92/06204) and region-directed mutagenesis (Derbyshire et al., 1986, Gene 46: 145; Ner et al., 1988, DNA 7: 127).

Mutagenesis/shuffling methods can be combined with high-throughput, automated screening methods to detect activity of cloned, mutagenized polypeptides expressed by host cells (Ness et al., 1999, Nature Biotechnology 17: 893-896). Mutagenized DNA molecules that encode active polypeptides can be recovered from the host cells and rapidly sequenced using standard methods in the art. These methods allow the rapid determination of the importance of individual amino acid residues in a polypeptide.

Semi-synthetic gene construction is accomplished by combining aspects of synthetic gene construction, and/or site-directed mutagenesis, and/or random mutagenesis, and/or shuffling. Semi-synthetic construction is typified by a process utilizing polynucleotide fragments that are synthesized, in combination with PCR techniques. Defined regions of genes may thus be synthesized de novo, while other regions may be amplified using site-specific mutagenic primers, while yet other regions may be subjected to error-prone PCR or non-error prone PCR amplification. Polynucleotide subsequences may then be shuffled.

### Polynucleotides

The present invention also relates to isolated polynucleotides encoding a variant of the present invention. Accordingly, the present invention relates to isolated polynucleotides encoding a variant comprising a substitution at one or more positions corresponding to positions 33 and 188 of the mature polypeptide of SEQ ID NO: 26 using the numbering of SEQ ID NO: 26, wherein the variant has beta-glucanase activity and wherein the variant has at least 60%, e.g., at least 65%, at least 70%, at least 75%, at least 80%, at least 85%, at least 90%, at least 95%, at least 95.5%, at least 96%, at least 96.5%, at least 97%, at least 97.5%, at least 98%, at least 98.5%, at least 99%, or at least 99.5%, but less than 100% sequence identity to the mature polypeptide of any of: SEQ ID NO: 26, SEQ ID NO: 27, SEQ ID NO: 25, and SEQ ID NO: 28.

### Nucleic Acid Constructs

The present invention also relates to nucleic acid constructs comprising a polynucleotide of the present invention operably linked to one or more control sequences that direct the expression of the coding sequence in a suitable host cell under conditions compatible with the control sequences. Accordingly, the present invention relates to nucleic acid constructs comprising a polynucleotide encoding a variant comprising a substitution at one or more positions corresponding to positions 33 and 188 of the mature polypeptide of SEQ ID NO: 26 using the numbering of SEQ ID NO: 26, wherein the variant has beta-glucanase activity and wherein the variant has at least 60%, e.g., at least 65%, at least 70%, at least 75%, at least 80%, at least 85%, at least 90%, at least 95%, at least 95.5%, at least 96%, at least 96.5%, at least 97%, at least 97.5%, at least 98%, at least 98.5%, at least 99%, or at least 99.5%, but less than 100% sequence identity to the mature polypeptide of any of: SEQ ID NO: 26, SEQ ID NO: 27, SEQ ID NO: 25, and SEQ ID NO: 28, wherein the polynucleotide is operately linked to one or more control sequences that direct the expression of the coding sequence in a suitable host cell under conditions compatible with the control sequences.

The polynucleotide may be manipulated in a variety of ways to provide for expression of the polypeptide. Manipulation of the polynucleotide prior to its insertion into a vector may be desirable or necessary depending on the expression vector. The techniques for modifying polynucleotides utilizing recombinant DNA methods are well known in the art.

The control sequence may be a promoter, a polynucleotide that is recognized by a host cell for expression of a polynucleotide encoding a polypeptide of the present invention. The promoter contains transcriptional control sequences that mediate the expression of the polypeptide. The promoter may be any polynucleotide that shows transcriptional activity in the host cell including variant, truncated, and hybrid promoters, and may be obtained from genes encoding extracellular or intracellular polypeptides either homologous or heterologous to the host cell.

Examples of suitable promoters for directing transcription of the nucleic acid constructs of the present invention in a bacterial host cell are the promoters obtained from the Bacillus amyloliquefaciens alpha-amylase gene (amyQ), Bacillus licheniformis alpha-amylase gene (amyL), Bacillus licheniformis penicillinase gene (penP), Bacillus stearothermophilus maltogenic amylase gene (amyM), Bacillus subtilis levansucrase gene (sacB), Bacillus subtilis xylA and xylB genes, Bacillus thuringiensis ctyIIIA gene (Agaisse and Lereclus, 1994, Molecular Microbiology 13: 97-107), E. coli lac operon, E. coli trc promoter (Egon et al., 1988, Gene 69: 301-315), Streptomyces coelicolor agarase gene (dagA), and prokaryotic beta-lactamase gene (Villa-Kamaroff et al., 1978, Proc. Natl. Acad. Sci. USA 75: 3727-3731), as well as the tac promoter (DeBoer et al., 1983, Proc. Natl. Acad. Sci. USA 80: 21-25). Further promoters are described in “Useful proteins from recombinant bacteria” in Gilbert et al., 1980, Scientific American 242: 74-94; and in Sambrook et al., 1989, supra. Examples of tandem promoters are disclosed in WO 99/43835.

Examples of suitable promoters for directing transcription of the nucleic acid constructs of the present invention in a filamentous fungal host cell are promoters obtained from the genes for Aspergillus nidulans acetamidase, Aspergillus niger neutral alpha-amylase, Aspergillus niger acid stable alpha-amylase, Aspergillus niger or Aspergillus awamori glucoamylase (glaA), Aspergillus oryzae TAKA amylase, Aspergillus oryzae alkaline protease, Aspergillus oryzae triose phosphate isomerase, Fusarium oxysporum trypsin-like protease (WO 96/00787), Fusarium venenatum amyloglucosidase (WO 00/56900), Fusarium venenatum Daria (WO 00/56900), Fusarium venenatum Quinn (WO 00/56900), Rhizomucor miehei lipase, Rhizomucor miehei aspartic proteinase, Trichoderma reesei beta-glucosidase, Trichoderma reesei cellobiohydrolase I, Trichoderma reesei cellobiohydrolase II, Trichoderma reesei endoglucanase I, Trichoderma reesei endoglucanase II, Trichoderma reesei endoglucanase III, Trichoderma reesei endoglucanase V, Trichoderma reesei xylanase I, Trichoderma reesei xylanase II, Trichoderma reesei xylanase III, Trichoderma reesei beta-xylosidase, and Trichoderma reesei translation elongation factor, as well as the NA2-tpi promoter (a modified promoter from an Aspergillus neutral alpha-amylase gene in which the untranslated leader has been replaced by an untranslated leader from an Aspergillus triose phosphate isomerase gene; non-limiting examples include modified promoters from an Aspergillus niger neutral alpha-amylase gene in which the untranslated leader has been replaced by an untranslated leader from an Aspergillus nidulans or Aspergillus oryzae triose phosphate isomerase gene); and variant, truncated, and hybrid promoters thereof. Other promoters are described in U.S. Pat. No. 6,011,147.

In a yeast host, useful promoters are obtained from the genes for Saccharomyces cerevisiae enolase (ENO-1), Saccharomyces cerevisiae galactokinase (GAL1), Saccharomyces cerevisiae alcohol dehydrogenase/glyceraldehyde-3-phosphate dehydrogenase (ADH1, ADH2/GAP), Saccharomyces cerevisiae triose phosphate isomerase (TPI), Saccharomyces cerevisiae metallothionein (CUP1), and Saccharomyces cerevisiae 3-phosphoglycerate kinase. Other useful promoters for yeast host cells are described by Romanos et al., 1992, Yeast 8: 423-488.

The control sequence may also be a transcription terminator, which is recognized by a host cell to terminate transcription. The terminator is operably linked to the 3′-terminus of the polynucleotide encoding the polypeptide. Any terminator that is functional in the host cell may be used in the present invention.

Preferred terminators for bacterial host cells are obtained from the genes for Bacillus clausii alkaline protease (aprH), Bacillus licheniformis alpha-amylase (amyL), and Escherichia coli ribosomal RNA (rrnB).

Preferred terminators for filamentous fungal host cells are obtained from the genes for Aspergillus nidulans acetamidase, Aspergillus nidulans anthranilate synthase, Aspergillus niger glucoamylase, Aspergillus niger alpha-glucosidase, Aspergillus oryzae TAKA amylase, Fusarium oxysporum trypsin-like protease, Trichoderma reesei beta-glucosidase, Trichoderma reesei cellobiohydrolase I, Trichoderma reesei cellobiohydrolase II, Trichoderma reesei endoglucanase I, Trichoderma reesei endoglucanase II, Trichoderma reesei endoglucanase III, Trichoderma reesei endoglucanase V, Trichoderma reesei xylanase I, Trichoderma reesei xylanase II, Trichoderma reesei xylanase III, Trichoderma reesei beta-xylosidase, and Trichoderma reesei translation elongation factor.

Preferred terminators for yeast host cells are obtained from the genes for Saccharomyces cerevisiae enolase, Saccharomyces cerevisiae cytochrome C (CYC1), and Saccharomyces cerevisiae glyceraldehyde-3-phosphate dehydrogenase. Other useful terminators for yeast host cells are described by Romanos et al., 1992, supra.

The control sequence may also be an mRNA stabilizer region downstream of a promoter and upstream of the coding sequence of a gene which increases expression of the gene.

Examples of suitable mRNA stabilizer regions are obtained from a Bacillus thuringiensis ctyIIIA gene (WO 94/25612) and a Bacillus subtilis SP82 gene (Hue et al., 1995, Journal of Bacteriology 177: 3465-3471).

The control sequence may also be a leader, a nontranslated region of an mRNA that is important for translation by the host cell. The leader is operably linked to the 5′-terminus of the polynucleotide encoding the polypeptide. Any leader that is functional in the host cell may be used.

Preferred leaders for filamentous fungal host cells are obtained from the genes for Aspergillus oryzae TAKA amylase and Aspergillus nidulans triose phosphate isomerase.

Suitable leaders for yeast host cells are obtained from the genes for Saccharomyces cerevisiae enolase (ENO-1), Saccharomyces cerevisiae 3-phosphoglycerate kinase, Saccharomyces cerevisiae alpha-factor, and Saccharomyces cerevisiae alcohol dehydrogenase/glyceraldehyde-3-phosphate dehydrogenase (ADH2/GAP).

The control sequence may also be a polyadenylation sequence, a sequence operably linked to the 3′-terminus of the polynucleotide and, when transcribed, is recognized by the host cell as a signal to add polyadenosine residues to transcribed mRNA. Any polyadenylation sequence that is functional in the host cell may be used.

Preferred polyadenylation sequences for filamentous fungal host cells are obtained from the genes for Aspergillus nidulans anthranilate synthase, Aspergillus niger glucoamylase, Aspergillus niger alpha-glucosidase Aspergillus oryzae TAKA amylase, and Fusarium oxysporum trypsin-like protease.

Useful polyadenylation sequences for yeast host cells are described by Guo and Sherman, 1995, Mol. Cellular Biol. 15: 5983-5990.

The control sequence may also be a signal peptide coding region that encodes a signal peptide linked to the N-terminus of a polypeptide and directs the polypeptide into the cell's secretory pathway. The 5′-end of the coding sequence of the polynucleotide may inherently contain a signal peptide coding sequence naturally linked in translation reading frame with the segment of the coding sequence that encodes the polypeptide. Alternatively, the 5′-end of the coding sequence may contain a signal peptide coding sequence that is foreign to the coding sequence. A foreign signal peptide coding sequence may be required where the coding sequence does not naturally contain a signal peptide coding sequence. Alternatively, a foreign signal peptide coding sequence may simply replace the natural signal peptide coding sequence in order to enhance secretion of the polypeptide. However, any signal peptide coding sequence that directs the expressed polypeptide into the secretory pathway of a host cell may be used.

Effective signal peptide coding sequences for bacterial host cells are the signal peptide coding sequences obtained from the genes for Bacillus NCIB 11837 maltogenic amylase, Bacillus licheniformis subtilisin, Bacillus licheniformis beta-lactamase, Bacillus stearothermophilus alpha-amylase, Bacillus stearothermophilus neutral proteases (nprT, nprS, nprM), and Bacillus subtilis prsA. Further signal peptides are described by Simonen and Palva, 1993, Microbiological Reviews 57: 109-137.

Effective signal peptide coding sequences for filamentous fungal host cells are the signal peptide coding sequences obtained from the genes for Aspergillus niger neutral amylase, Aspergillus niger glucoamylase, Aspergillus oryzae TAKA amylase, Humicola insolens cellulase, Humicola insolens endoglucanase V, Humicola lanuginosa lipase, and Rhizomucor miehei aspartic proteinase.

Useful signal peptides for yeast host cells are obtained from the genes for Saccharomyces cerevisiae alpha-factor and Saccharomyces cerevisiae invertase. Other useful signal peptide coding sequences are described by Romanos et al., 1992, supra.

The control sequence may also be a propeptide coding sequence that encodes a propeptide positioned at the N-terminus of a polypeptide. The resultant polypeptide is known as a proenzyme or propolypeptide (or a zymogen in some cases). A propolypeptide is generally inactive and can be converted to an active polypeptide by catalytic or autocatalytic cleavage of the propeptide from the propolypeptide. The propeptide coding sequence may be obtained from the genes for Bacillus subtilis alkaline protease (aprE), Bacillus subtilis neutral protease (nprT), Myceliophthora thermophila laccase (WO 95/33836), Rhizomucor miehei aspartic proteinase, and Saccharomyces cerevisiae alpha-factor.

Where both signal peptide and propeptide sequences are present, the propeptide sequence is positioned next to the N-terminus of a polypeptide and the signal peptide sequence is positioned next to the N-terminus of the propeptide sequence.

It may also be desirable to add regulatory sequences that regulate expression of the polypeptide relative to the growth of the host cell. Examples of regulatory sequences are those that cause expression of the gene to be turned on or off in response to a chemical or physical stimulus, including the presence of a regulatory compound. Regulatory sequences in prokaryotic systems include the lac, tac, and trp operator systems. In yeast, the ADH2 system or GAL1 system may be used. In filamentous fungi, the Aspergillus niger glucoamylase promoter, Aspergillus oryzae TAKA alpha-amylase promoter, and Aspergillus oryzae glucoamylase promoter, Trichoderma reesei cellobiohydrolase I promoter, and Trichoderma reesei cellobiohydrolase II promoter may be used. Other examples of regulatory sequences are those that allow for gene amplification. In eukaryotic systems, these regulatory sequences include the dihydrofolate reductase gene that is amplified in the presence of methotrexate, and the metallothionein genes that are amplified with heavy metals. In these cases, the polynucleotide encoding the polypeptide would be operably linked to the regulatory sequence.

### Expression Vectors

The present invention also relates to recombinant expression vectors comprising a polynucleotide of the present invention, a promoter, and transcriptional and translational stop signals. Accordingly, the present invention relates to recombinant expression vectors comprising a polynucleotide encoding a variant comprising a substitution at one or more positions corresponding to positions 33 and 188 of the mature polypeptide of SEQ ID NO: 26 using the numbering of SEQ ID NO: 26, wherein the variant has beta-glucanase activity and wherein the variant has at least 60%, e.g., at least 65%, at least 70%, at least 75%, at least 80%, at least 85%, at least 90%, at least 95%, at least 95.5%, at least 96%, at least 96.5%, at least 97%, at least 97.5%, at least 98%, at least 98.5%, at least 99%, or at least 99.5%, but less than 100% sequence identity to the mature polypeptide of any of: SEQ ID NO: 26, SEQ ID NO: 27, SEQ ID NO: 25, and SEQ ID NO: 28, a promoter, and transcriptional and translational stop signals.

The various nucleotide and control sequences may be joined together to produce a recombinant expression vector that may include one or more convenient restriction sites to allow for insertion or substitution of the polynucleotide encoding the polypeptide at such sites. Alternatively, the polynucleotide may be expressed by inserting the polynucleotide or a nucleic acid construct comprising the polynucleotide into an appropriate vector for expression. In creating the expression vector, the coding sequence is located in the vector so that the coding sequence is operably linked with the appropriate control sequences for expression.

The recombinant expression vector may be any vector (e.g., a plasmid or virus) that can be conveniently subjected to recombinant DNA procedures and can bring about expression of the polynucleotide. The choice of the vector will typically depend on the compatibility of the vector with the host cell into which the vector is to be introduced. The vector may be a linear or closed circular plasmid.

The vector may be an autonomously replicating vector, i.e., a vector that exists as an extrachromosomal entity, the replication of which is independent of chromosomal replication, e.g., a plasmid, an extrachromosomal element, a minichromosome, or an artificial chromosome. The vector may contain any means for assuring self-replication. Alternatively, the vector may be one that, when introduced into the host cell, is integrated into the genome and replicated together with the chromosome(s) into which it has been integrated. Furthermore, a single vector or plasmid or two or more vectors or plasmids that together contain the total DNA to be introduced into the genome of the host cell, or a transposon, may be used.

The vector preferably contains one or more selectable markers that permit easy selection of transformed, transfected, transduced, or the like cells. A selectable marker is a gene the product of which provides for biocide or viral resistance, resistance to heavy metals, prototrophy to auxotrophs, and the like.

Examples of bacterial selectable markers are Bacillus licheniformis or Bacillus subtilis dal genes, or markers that confer antibiotic resistance such as ampicillin, chloramphenicol, kanamycin, neomycin, spectinomycin, or tetracycline resistance. Suitable markers for yeast host cells include, but are not limited to, ADE2, HIS3, LEU2, LYS2, MET3, TRP1, and URA3. Selectable markers for use in a filamentous fungal host cell include, but are not limited to, adeA (phosphoribosylaminoimidazole-succinocarboxamide synthase), adeB (phosphoribosyl-aminoimidazole synthase), amdS (acetamidase), argB (ornithine carbamoyltransferase), bar (phosphinothricin acetyltransferase), hph (hygromycin phosphotransferase), niaD (nitrate reductase), pyrG (orotidine-5′-phosphate decarboxylase), sC (sulfate adenyltransferase), and trpC (anthranilate synthase), as well as equivalents thereof. Preferred for use in an Aspergillus cell are Aspergillus nidulans or Aspergillus oryzae amdS and pyrG genes and a Streptomyces hygroscopicus bar gene. Preferred for use in a Trichoderma cell are adeA, adeB, amdS, hph, and pyrG genes.

The selectable marker may be a dual selectable marker system as described in WO 2010/039889. In one aspect, the dual selectable marker is an hph-tk dual selectable marker system.

The vector preferably contains an element(s) that permits integration of the vector into the host cell's genome or autonomous replication of the vector in the cell independent of the genome.

For integration into the host cell genome, the vector may rely on the polynucleotide's sequence encoding the polypeptide or any other element of the vector for integration into the genome by homologous or non-homologous recombination. Alternatively, the vector may contain additional polynucleotides for directing integration by homologous recombination into the genome of the host cell at a precise location(s) in the chromosome(s). To increase the likelihood of integration at a precise location, the integrational elements should contain a sufficient number of nucleic acids, such as 100 to 10,000 base pairs, 400 to 10,000 base pairs, and 800 to 10,000 base pairs, which have a high degree of sequence identity to the corresponding target sequence to enhance the probability of homologous recombination. The integrational elements may be any sequence that is homologous with the target sequence in the genome of the host cell. Furthermore, the integrational elements may be non-encoding or encoding polynucleotides. On the other hand, the vector may be integrated into the genome of the host cell by non-homologous recombination.

For autonomous replication, the vector may further comprise an origin of replication enabling the vector to replicate autonomously in the host cell in question. The origin of replication may be any plasmid replicator mediating autonomous replication that functions in a cell. The term “origin of replication” or “plasmid replicator” means a polynucleotide that enables a plasmid or vector to replicate in vivo.

Examples of bacterial origins of replication are the origins of replication of plasmids pBR322, pUC19, pACYC177, and pACYC184 permitting replication in E. coli, and pUB110, pE194, pTA1060, and 4ß1 permitting replication in Bacillus.

Examples of origins of replication for use in a yeast host cell are the 2 micron origin of replication, ARS1, ARS4, the combination of ARS1 and CEN3, and the combination of ARS4 and CEN6.

Examples of origins of replication useful in a filamentous fungal cell are AMA1 and ANS1 (Gems et al., 1991, Gene 98: 61-67; Cullen et al., 1987, Nucleic Acids Res. 15: 9163-9175; WO 00/24883). Isolation of the AMA1 gene and construction of plasmids or vectors comprising the gene can be accomplished according to the methods disclosed in WO 00/24883.

More than one copy of a polynucleotide of the present invention may be inserted into a host cell to increase production of a polypeptide. An increase in the copy number of the polynucleotide can be obtained by integrating at least one additional copy of the sequence into the host cell genome or by including an amplifiable selectable marker gene with the polynucleotide where cells containing amplified copies of the selectable marker gene, and thereby additional copies of the polynucleotide, can be selected for by cultivating the cells in the presence of the appropriate selectable agent.

The procedures used to ligate the elements described above to construct the recombinant expression vectors of the present invention are well known to one skilled in the art (see, e.g., Sambrook et al., 1989, supra).

### Host Cells

The present invention also relates to recombinant host cells, comprising a polynucleotide of the present invention operably linked to one or more control sequences that direct the production of a polypeptide of the present invention. Accordingly, the present invention relates to recombinant host cells, comprising a polynucleotide encoding a variant comprising a substitution at one or more positions corresponding to positions 33 and 188 of the mature polypeptide of SEQ ID NO: 26 using the numbering of SEQ ID NO: 26, wherein the variant has beta-glucanase activity and wherein the variant has at least 60%, e.g., at least 65%, at least 70%, at least 75%, at least 80%, at least 85%, at least 90%, at least 95%, at least 95.5%, at least 96%, at least 96.5%, at least 97%, at least 97.5%, at least 98%, at least 98.5%, at least 99%, or at least 99.5%, but less than 100% sequence identity to the mature polypeptide of any of: SEQ ID NO: 26, SEQ ID NO: 27, SEQ ID NO: 25, and SEQ ID NO: 28, wherein the polynucleotide is operably linked to one or more control sequences that direct the production of a polypeptide encoding the variant.

A construct or vector comprising a polynucleotide is introduced into a host cell so that the construct or vector is maintained as a chromosomal integrant or as a self-replicating extra-chromosomal vector as described earlier. The term “host cell” encompasses any progeny of a parent cell that is not identical to the parent cell due to mutations that occur during replication. The choice of a host cell will to a large extent depend upon the gene encoding the polypeptide and its source.

The host cell may be any cell useful in the recombinant production of a polypeptide of the present invention, e.g., a prokaryote or a eukaryote.

The prokaryotic host cell may be any Gram-positive or Gram-negative bacterium. Gram-positive bacteria include, but are not limited to, Bacillus, Clostridium, Enterococcus, Geobacillus, Lactobacillus, Lactococcus, Oceanobacillus, Staphylococcus, Streptococcus, and Streptomyces. Gram-negative bacteria include, but are not limited to, Campylobacter, E. coli, Flavobacterium, Fusobacterium, Helicobacter, Ilyobacter, Neisseria, Pseudomonas, Salmonella, and Ureaplasma.

The bacterial host cell may be any Bacillus cell including, but not limited to, Bacillus alkalophilus, Bacillus amyloliquefaciens, Bacillus brevis, Bacillus circulans, Bacillus clausii, Bacillus coagulans, Bacillus firmus, Bacillus lautus, Bacillus lentus, Bacillus licheniformis, Bacillus megaterium, Bacillus pumilus, Bacillus stearothermophilus, Bacillus subtilis, Bacillus sp-62449, Bacillus akibai, Bacillus agaradhaerens, Bacillus mojavensis and Bacillus thuringiensis cells.

The bacterial host cell may also be any Streptococcus cell including, but not limited to, Streptococcus equisimilis, Streptococcus pyogenes, Streptococcus uberis, and Streptococcusequi subsp. Zooepidemicus cells.

The bacterial host cell may also be any Streptomyces cell including, but not limited to, Streptomyces achromogenes, Streptomyces avermitilis, Streptomyces coelicolor, Streptomyces griseus, and Streptomyces lividans cells.

The introduction of DNA into a Bacillus cell may be effected by protoplast transformation (see, e.g., Chang and Cohen, 1979, Mol. Gen. Genet. 168: 111-115), competent cell transformation (see, e.g., Young and Spizizen, 1961, J. Bacteriol. 81: 823-829, or Dubnau and Davidoff-Abelson, 1971, J. Mol. Biol. 56: 209-221), electroporation (see, e.g., Shigekawa and Dower, 1988, Biotechniques 6: 742-751), or conjugation (see, e.g., Koehler and Thorne, 1987, J. Bacteriol. 169: 5271-5278). The introduction of DNA into an E. coli cell may be effected by protoplast transformation (see, e.g., Hanahan, 1983, J. Mol. Biol. 166: 557-580) or electroporation (see, e.g., Dower et al., 1988, Nucleic Acids Res. 16: 6127-6145). The introduction of DNA into a Streptomyces cell may be effected by protoplast transformation, electroporation (see, e.g., Gong et al., 2004, Folia Microbiol. (Praha) 49: 399-405), conjugation (see, e.g., Mazodier et al., 1989, J. Bacteriol. 171: 3583-3585), or transduction (see, e.g., Burke et al., 2001, Proc. Natl. Acad. Sci. USA 98: 6289-6294). The introduction of DNA into a Pseudomonas cell may be effected by electroporation (see, e.g., Choi et al., 2006, J. Microbiol. Methods 64: 391-397) or conjugation (see, e.g., Pinedo and Smets, 2005, Appl. Environ. Microbiol. 71: 51-57). The introduction of DNA into a Streptococcus cell may be effected by natural competence (see, e.g., Perry and Kuramitsu, 1981, Infect. Immun. 32: 1295-1297), protoplast transformation (see, e.g., Catt and Jollick, 1991, Microbios 68: 189-207), electroporation (see, e.g., Buckley et al., 1999, Appl. Environ. Microbiol. 65: 3800-3804), or conjugation (see, e.g., Clewell, 1981, Microbiol. Rev. 45: 409-436). However, any method known in the art for introducing DNA into a host cell can be used.

The host cell may also be a eukaryote, such as a mammalian, insect, plant, or fungal cell.

The host cell may be a fungal cell. “Fungi” as used herein includes the phyla Ascomycota, Basidiomycota, Chytridiomycota, and Zygomycota as well as the Oomycota and all mitosporic fungi (as defined by Hawksworth et al., In, Ainsworth and Bisby's Dictionary of The Fungi, 8th edition, 1995, CAB International, University Press, Cambridge, UK).

The fungal host cell may be a yeast cell. “Yeast” as used herein includes ascosporogenous yeast (Endomycetales), basidiosporogenous yeast, and yeast belonging to the Fungi Imperfecti (Blastomycetes). Since the classification of yeast may change in the future, for the purposes of this invention, yeast shall be defined as described in Biology and Activities of Yeast (Skinner, Passmore, and Davenport, editors, Soc. App. Bacteriol. Symposium Series No. 9, 1980).

The yeast host cell may be a Candida, Hansenula, Kluyveromyces, Pichia, Saccharomyces, Schizosaccharomyces, or Yarrowia cell, such as a Kluyveromyces lactis, Saccharomyces carlsbergensis, Saccharomyces cerevisiae, Saccharomyces diastaticus, Saccharomyces douglasii, Saccharomyces kluyveri, Saccharomyces norbensis, Saccharomyces oviformis, or Yarrowia lipolytica cell.

The fungal host cell may be a filamentous fungal cell. “Filamentous fungi” include all filamentous forms of the subdivision Eumycota and Oomycota (as defined by Hawksworth et al., 1995, supra). The filamentous fungi are generally characterized by a mycelial wall composed of chitin, cellulose, glucan, chitosan, mannan, and other complex polysaccharides. Vegetative growth is by hyphal elongation and carbon catabolism is obligately aerobic. In contrast, vegetative growth by yeasts such as Saccharomyces cerevisiae is by budding of a unicellular thallus and carbon catabolism may be fermentative.

The filamentous fungal host cell may be an Acremonium, Aspergillus, Aureobasidium, Bjerkandera, Ceriporiopsis, Chrysosporium, Coprinus, Coriolus, Cryptococcus, Filibasidium, Fusarium, Humicola, Magnaporthe, Mucor, Myceliophthora, Neocallimastix, Neurospora, Paecilomyces, Penicillium, Phanerochaete, Phlebia, Piromyces, Pleurotus, Schizophyllum, Talaromyces, Thermoascus, Thielavia, Tolypocladium, Trametes, or Trichoderma cell.

For example, the filamentous fungal host cell may be an Aspergillus awamori, Aspergillus foetidus, Aspergillus fumigatus, Aspergillus japonicus, Aspergillus nidulans, Aspergillus niger, Aspergillus oryzae, Bjerkandera adusta, Ceriporiopsis aneirina, Ceriporiopsis caregiea, Ceriporiopsis gilvescens, Ceriporiopsis pannocinta, Ceriporiopsis rivulosa, Ceriporiopsis subrufa, Ceriporiopsis subvermispora, Chrysosporium inops, Chrysosporium keratinophilum, Chrysosporium lucknowense, Chrysosporium merdarium, Chrysosporium pannicola, Chrysosporium queenslandicum, Chrysosporium tropicum, Chrysosporium zonatum, Coprinus cinereus, Coriolus hirsutus, Fusarium bactridioides, Fusarium cerealis, Fusarium crookwellense, Fusarium culmorum, Fusarium graminearum, Fusarium graminum, Fusarium heterosporum, Fusarium negundi, Fusarium oxysporum, Fusarium reticulatum, Fusarium roseum, Fusarium sambucinum, Fusarium sarcochroum, Fusarium sporotrichioides, Fusarium sulphureum, Fusarium torulosum, Fusarium trichothecioides, Fusarium venenatum, Humicola insolens, Humicola lanuginosa, Mucor miehei, Myceliophthora thermophila, Neurospora crassa, Penicillium purpurogenum, Phanerochaete chrysosporium, Phlebia radiata, Pleurotus eryngii, Thielavia terrestris, Trametes villosa, Trametes versicolor, Trichoderma harzianum, Trichoderma koningii, Trichoderma longibrachiatum, Trichoderma reesei, or Trichoderma viride cell.

Fungal cells may be transformed by a process involving protoplast formation, transformation of the protoplasts, and regeneration of the cell wall in a manner known per se. Suitable procedures for transformation of Aspergillus and Trichoderma host cells are described in EP 238023, Yelton et al., 1984, Proc. Natl. Acad. Sci. USA 81: 1470-1474, and Christensen et al., 1988, Bio/Technology 6: 1419-1422. Suitable methods for transforming Fusarium species are described by Malardier et al., 1989, Gene 78: 147-156, and WO 96/00787. Yeast may be transformed using the procedures described by Becker and Guarente, In Abelson, J. N. and Simon, M. I., editors, Guide to Yeast Genetics and Molecular Biology, Methods in Enzymology, Volume 194, pp 182-187, Academic Press, Inc., New York; Ito et al., 1983, J. Bacteriol. 153: 163; and Hinnen et al., 1978, Proc. Natl. Acad. Sci. USA 75: 1920.

### Methods of Production

The present invention also relates to methods of producing a variant (e.g., in vitro or ex vivo methods of production), comprising: (a) cultivating a host cell of the present invention under conditions suitable for expression of the variant; and (b) recovering the variant. Accordingly, the present invention relates to methods of producing a variant comprising a substitution at one or more positions corresponding to positions 33 and 188 of the mature polypeptide of SEQ ID NO: 26 using the numbering of SEQ ID NO: 26, wherein the variant has beta-glucanase activity and wherein the variant has at least 60%, e.g., at least 65%, at least 70%, at least 75%, at least 80%, at least 85%, at least 90%, at least 95%, at least 95.5%, at least 96%, at least 96.5%, at least 97%, at least 97.5%, at least 98%, at least 98.5%, at least 99%, or at least 99.5%, but less than 100% sequence identity to the mature polypeptide of any of: SEQ ID NO: 26, SEQ ID NO: 27, SEQ ID NO: 25, and SEQ ID NO: 28, wherein the method comprises (a) cultivating a host cell expressing the variant under conditions suitable for expression of the variant, and (b) recovering the variant.

In one aspect, the cell is a Bacillus cell. In another aspect, the cell is a B. subtilis, B. licheniformis, Bacillus sp-62449, or Bacillus akibai, or Bacillus agaradhaerens, or Bacillus mojavensis cell.

The host cells are cultivated in a nutrient medium suitable for production of the variant using methods known in the art. For example, the cell may be cultivated by shake flask cultivation, or small-scale or large-scale fermentation (including continuous, batch, fed-batch, or solid state fermentations) in laboratory or industrial fermentors performed in a suitable medium and under conditions allowing the variant to be expressed and/or isolated. The cultivation takes place in a suitable nutrient medium comprising carbon and nitrogen sources and inorganic salts, using procedures known in the art. Suitable media are available from commercial suppliers or may be prepared according to published compositions (e.g., in catalogues of the American Type Culture Collection). If the variant is secreted into the nutrient medium, the variant can be recovered directly from the medium. If the variant is not secreted, it can be recovered from cell lysates.

The variant may be detected using methods known in the art that are specific for the variants These detection methods include, but are not limited to, use of specific antibodies, formation of an enzyme product, or disappearance of an enzyme substrate. For example, an enzyme assay may be used to determine the activity of the variant.

The variant may be recovered using methods known in the art. For example, the variant may be recovered from the nutrient medium by conventional procedures including, but not limited to, collection, centrifugation, filtration, extraction, spray-drying, evaporation, or precipitation.

The variant may be purified by a variety of procedures known in the art including, but not limited to, chromatography (e.g., ion exchange, affinity, hydrophobic, chromatofocusing, and size exclusion), electrophoretic procedures (e.g., preparative isoelectric focusing), differential solubility (e.g., ammonium sulfate precipitation), SDS-PAGE, or extraction (see, e.g., Protein Purification, Janson and Ryden, editors, VCH Publishers, New York, 1989) to obtain substantially pure variants.

In an alternative aspect, the variant is not recovered, but rather a host cell of the present invention expressing the variant is used as a source of the variant.

### Production in Plants

The present invention also relates to plants, e.g., a transgenic plant, plant part, or plant cell, comprising a polynucleotide of the present invention so as to express and produce the variant in recoverable quantities. Accordingly, the present invention relates to plants, comprising a polynucleotide encoding a variant comprising a substitution at one or more positions corresponding to positions 33 and 188 of the mature polypeptide of SEQ ID NO: 26 using the numbering of SEQ ID NO: 26, wherein the variant has beta-glucanase activity and wherein the variant has at least 60%, e.g., at least 65%, at least 70%, at least 75%, at least 80%, at least 85%, at least 90%, at least 95%, at least 95.5%, at least 96%, at least 96.5%, at least 97%, at least 97.5%, at least 98%, at least 98.5%, at least 99%, or at least 99.5%, but less than 100% sequence identity to the mature polypeptide of any of: SEQ ID NO: 26, SEQ ID NO: 27, SEQ ID NO: 25, and SEQ ID NO: 28, wherein the plant express and produce the variant in recoverable quantities.

The variant may be recovered from the plant or plant part. Alternatively, the plant or plant part containing the variant may be used as such for improving the quality of a food or feed, e.g., improving nutritional value, palatability, and rheological properties, or to destroy an antinutritive factor.

The transgenic plant can be dicotyledonous (a dicot) or monocotyledonous (a monocot). Examples of monocot plants are grasses, such as meadow grass (blue grass, Poa), forage grass such as Festuca, Lolium, temperate grass, such as Agrostis, and cereals, e.g., wheat, oats, rye, barley, rice, sorghum, and maize (corn).

Examples of dicot plants are tobacco, legumes, such as lupins, potato, sugar beet, pea, bean and soybean, and cruciferous plants (family Brassicaceae), such as cauliflower, rape seed, and the closely related model organism Arabidopsis thaliana.

Examples of plant parts are stem, callus, leaves, root, fruits, seeds, and tubers as well as the individual tissues comprising these parts, e.g., epidermis, mesophyll, parenchyme, vascular tissues, meristems. Specific plant cell compartments, such as chloroplasts, apoplasts, mitochondria, vacuoles, peroxisomes and cytoplasm are also considered to be a plant part. Furthermore, any plant cell, whatever the tissue origin, is considered to be a plant part. Likewise, plant parts such as specific tissues and cells isolated to facilitate the utilization of the invention are also considered plant parts, e.g., embryos, endosperms, aleurone and seed coats.

Also included within the scope of the present invention are the progeny of such plants, plant parts, and plant cells.

The transgenic plant or plant cell expressing a variant may be constructed in accordance with methods known in the art. In short, the plant or plant cell is constructed by incorporating one or more expression constructs encoding a variant into the plant host genome or chloroplast genome and propagating the resulting modified plant or plant cell into a transgenic plant or plant cell.

The expression construct is conveniently a nucleic acid construct that comprises a polynucleotide encoding a variant operably linked with appropriate regulatory sequences required for expression of the polynucleotide in the plant or plant part of choice. Furthermore, the expression construct may comprise a selectable marker useful for identifying plant cells into which the expression construct has been integrated and DNA sequences necessary for introduction of the construct into the plant in question (the latter depends on the DNA introduction method to be used).

The choice of regulatory sequences, such as promoter and terminator sequences and optionally signal or transit sequences, is determined, for example, on the basis of when, where, and how the variant is desired to be expressed. For instance, the expression of the gene encoding a variant may be constitutive or inducible, or may be developmental, stage or tissue specific, and the gene product may be targeted to a specific tissue or plant part such as seeds or leaves. Regulatory sequences are, for example, described by Tague et al., 1988, Plant Physiology 86: 506.

For constitutive expression, the 35S-CaMV, the maize ubiquitin 1, or the rice actin 1 promoter may be used (Franck et al., 1980, Cell 21: 285-294; Christensen et al., 1992, Plant Mol. Biol. 18: 675-689; Zhang et al., 1991, Plant Cell 3: 1155-1165). Organ-specific promoters may be, for example, a promoter from storage sink tissues such as seeds, potato tubers, and fruits (Edwards and Coruzzi, 1990, Ann. Rev. Genet. 24: 275-303), or from metabolic sink tissues such as meristems (Ito et al., 1994, Plant Mol. Biol. 24: 863-878), a seed specific promoter such as the glutelin, prolamin, globulin, or albumin promoter from rice (Wu et al., 1998, Plant Cell Physiol. 39: 885-889), a Vicia faba promoter from the legumin B4 and the unknown seed protein gene from Vicia faba (Conrad et al., 1998, J. Plant Physiol. 152: 708-711), a promoter from a seed oil body protein (Chen et al., 1998, Plant Cell Physiol. 39: 935-941), the storage protein napA promoter from Brassica napus, or any other seed specific promoter known in the art, e.g., as described in WO 91/14772. Furthermore, the promoter may be a leaf specific promoter such as the rbcs promoter from rice or tomato (Kyozuka et al., 1993, Plant Physiol. 102: 991-1000), the chlorella virus adenine methyltransferase gene promoter (Mitra and Higgins, 1994, Plant Mol. Biol. 26: 85-93), the aldP gene promoter from rice (Kagaya et al., 1995, Mol. Gen. Genet. 248: 668-674), or a wound inducible promoter such as the potato pin2 promoter (Xu et al., 1993, Plant Mol. Biol. 22: 573-588). Likewise, the promoter may be induced by abiotic treatments such as temperature, drought, or alterations in salinity or induced by exogenously applied substances that activate the promoter, e.g., ethanol, oestrogens, plant hormones such as ethylene, abscisic acid, and gibberellic acid, and heavy metals.

A promoter enhancer element may also be used to achieve higher expression of a variant in the plant. For instance, the promoter enhancer element may be an intron that is placed between the promoter and the polynucleotide encoding a variant. For instance, Xu et al., 1993, supra, disclose the use of the first intron of the rice actin 1 gene to enhance expression.

The selectable marker gene and any other parts of the expression construct may be chosen from those available in the art.

The nucleic acid construct is incorporated into the plant genome according to conventional techniques known in the art, including Agrobacterium-mediated transformation, virus-mediated transformation, microinjection, particle bombardment, biolistic transformation, and electroporation (Gasser et al., 1990, Science 244: 1293; Potrykus, 1990, Bio/Technology 8: 535; Shimamoto et al., 1989, Nature 338: 274).

Agrobacterium tumefaciens-mediated gene transfer is a method for generating transgenic dicots (for a review, see Hooykas and Schilperoort, 1992, Plant Mol. Biol. 19: 15-38) and for transforming monocots, although other transformation methods may be used for these plants. A method for generating transgenic monocots is particle bombardment (microscopic gold or tungsten particles coated with the transforming DNA) of embryonic calli or developing embryos (Christou, 1992, Plant J. 2: 275-281; Shimamoto, 1994, Curr. Opin. Biotechnol. 5: 158-162; Vasil et al., 1992, Bio/Technology 10: 667-674). An alternative method for transformation of monocots is based on protoplast transformation as described by Omirulleh et al., 1993, Plant Mol. Biol. 21: 415-428. Additional transformation methods include those described in U.S. Pat. Nos. 6,395,966 and 7,151,204 (both of which are herein incorporated by reference in their entirety).

Following transformation, the transformants having incorporated the expression construct are selected and regenerated into whole plants according to methods well known in the art. Often the transformation procedure is designed for the selective elimination of selection genes either during regeneration or in the following generations by using, for example, co-transformation with two separate T-DNA constructs or site specific excision of the selection gene by a specific recombinase.

In addition to direct transformation of a particular plant genotype with a construct of the present invention, transgenic plants may be made by crossing a plant having the construct to a second plant lacking the construct. For example, a construct encoding a variant can be introduced into a particular plant variety by crossing, without the need for ever directly transforming a plant of that given variety. Therefore, the present invention encompasses not only a plant directly regenerated from cells which have been transformed in accordance with the present invention, but also the progeny of such plants. As used herein, progeny may refer to the offspring of any generation of a parent plant prepared in accordance with the present invention. Such progeny may include a DNA construct prepared in accordance with the present invention. Crossing results in the introduction of a transgene into a plant line by cross pollinating a starting line with a donor plant line. Non-limiting examples of such steps are described in U.S. Pat. No. 7,151,204.

Plants may be generated through a process of backcross conversion. For example, plants include plants referred to as a backcross converted genotype, line, inbred, or hybrid.

Genetic markers may be used to assist in the introgression of one or more transgenes of the invention from one genetic background into another. Marker assisted selection offers advantages relative to conventional breeding in that it can be used to avoid errors caused by phenotypic variations. Further, genetic markers may provide data regarding the relative degree of elite germplasm in the individual progeny of a particular cross. For example, when a plant with a desired trait which otherwise has a non-agronomically desirable genetic background is crossed to an elite parent, genetic markers may be used to select progeny which not only possess the trait of interest, but also have a relatively large proportion of the desired germplasm. In this way, the number of generations required to introgress one or more traits into a particular genetic background is minimized.

The present invention also relates to methods of producing a variant of the present invention comprising: (a) cultivating a transgenic plant or a plant cell comprising a polynucleotide encoding the variant under conditions conducive for production of the variant; and (b) recovering the variant.

### Fermentation Broth Formulations

The present invention also relates to a fermentation broth formulation comprising a polypeptide of the present invention (e.g. a variant of the present invention). Accordingly, the present invention relates to a fermentation broth formulation comprising a polypeptide encoding a variant comprising a substitution at one or more positions corresponding to positions 33 and 188 of the mature polypeptide of SEQ ID NO: 26 using the numbering of SEQ ID NO: 26, wherein the variant has beta-glucanase activity and wherein the variant has at least 60%, e.g., at least 65%, at least 70%, at least 75%, at least 80%, at least 85%, at least 90%, at least 95%, at least 95.5%, at least 96%, at least 96.5%, at least 97%, at least 97.5%, at least 98%, at least 98.5%, at least 99%, or at least 99.5%, but less than 100% sequence identity to the mature polypeptide of any of: SEQ ID NO: 26, SEQ ID NO: 27, SEQ ID NO: 25, and SEQ ID NO: 28.

The fermentation broth product further comprises additional ingredients used in the fermentation process, such as, for example, cells (including, the host cells containing the gene encoding the polypeptide of the present invention (e.g. a variant of the present invention) which are used to produce the polypeptide of interest), cell debris, biomass, fermentation media and/or fermentation products. In some embodiments, the composition is a cell-killed fermentation broth containing organic acid(s), killed cells and/or cell debris, and culture medium.

The term “fermentation broth” as used herein refers to a preparation produced by cellular fermentation that undergoes no or minimal recovery and/or purification. For example, fermentation broths are produced when microbial cultures are grown to saturation, incubated under carbon-limiting conditions to allow protein synthesis (e.g., expression of enzymes by host cells) and secretion into cell culture medium. The fermentation broth can contain unfractionated or fractionated contents of the fermentation materials derived at the end of the fermentation. Typically, the fermentation broth is unfractionated and comprises the spent culture medium and cell debris present after the microbial cells (e.g., filamentous fungal cells) are removed, e.g., by centrifugation. In some embodiments, the fermentation broth contains spent cell culture medium, extracellular enzymes, and viable and/or nonviable microbial cells.

In an embodiment, the fermentation broth formulation and cell compositions comprise a first organic acid component comprising at least one 1-5 carbon organic acid and/or a salt thereof and a second organic acid component comprising at least one 6 or more carbon organic acid and/or a salt thereof. In a specific embodiment, the first organic acid component is acetic acid, formic acid, propionic acid, a salt thereof, or a mixture of two or more of the foregoing and the second organic acid component is benzoic acid, cyclohexanecarboxylic acid, 4-methylvaleric acid, phenylacetic acid, a salt thereof, or a mixture of two or more of the foregoing.

In one aspect, the composition comprises an organic acid(s), and optionally further contains killed cells and/or cell debris. In one embodiment, the killed cells and/or cell debris are removed from a cell-killed fermentation broth to provide a composition that is free of these components.

The fermentation broth formulations or cell compositions may further comprise a preservative and/or anti-microbial (e.g., bacteriostatic) agent, including, but not limited to, sorbitol, sodium chloride, potassium sorbate, and others known in the art.

The cell-killed fermentation broth or composition may comprise the unfractionated contents of the fermentation materials derived at the end of the fermentation. Typically, the cell-killed fermentation broth or composition comprises the spent culture medium and cell debris present after the microbial cells (e.g., filamentous fungal cells) are grown to saturation, incubated under carbon-limiting conditions to allow protein synthesis. In some embodiments, the cell-killed fermentation broth or composition contains the spent cell culture medium, extracellular enzymes, and killed filamentous fungal cells. In some embodiments, the microbial cells present in the cell-killed fermentation broth or composition can be permeabilized and/or lysed using methods known in the art.

A fermentation broth as described herein is typically a liquid, but may comprise insoluble components, such as killed cells, cell debris, culture media components, and/or insoluble enzyme(s). In some embodiments, insoluble components may be removed to provide a clarified liquid composition.

The fermentation broth formulations and cell compositions of the present invention may be produced by a method described in WO 90/15861 or WO 2010/096673.

### Enzyme Compositions

The present invention also relates to compositions comprising a polypeptide of the present invention (e.g. a variant of the present invention). Accordingly, the present invention relates to compositions comprising a variant comprising a substitution at one or more positions corresponding to positions 33 and 188 of the mature polypeptide of SEQ ID NO: 26 using the numbering of SEQ ID NO: 26, wherein the variant has beta-glucanase activity and wherein the variant has at least 60%, e.g., at least 65%, at least 70%, at least 75%, at least 80%, at least 85%, at least 90%, at least 95%, at least 95.5%, at least 96%, at least 96.5%, at least 97%, at least 97.5%, at least 98%, at least 98.5%, at least 99%, or at least 99.5%, but less than 100% sequence identity to the mature polypeptide of any of: SEQ ID NO: 26, SEQ ID NO: 27, SEQ ID NO: 25, and SEQ ID NO: 28.

An embodiment is a cleaning or detergent composition comprising a beta-glucanase polypeptide of the invention (e.g. a variant of the present invention) and one or more amylases. Preferably, the compositions are enriched in such a polypeptide. The term “enriched” indicates that the beta-glucanase activity of the composition has been increased, e.g., with an enrichment factor of at least 1.1.

The compositions may comprise a polypeptide of the present invention (e.g. a variant of the present invention) as the major enzymatic component, e.g., a mono-component composition. Alternatively, the compositions may comprise multiple enzymatic activities, such as one or more (e.g., several) enzymes selected from the group consisting of hydrolase, isomerase, ligase, lyase, oxidoreductase, or transferase, e.g., an alpha-galactosidase, alpha-glucosidase, aminopeptidase, amylase, beta-galactosidase, beta-glucosidase, beta-xylosidase, carbohydrase, carboxypeptidase, catalase, cellobiohydrolase, cellulase, chitinase, cutinase, cyclodextrin glycosyltransferase, deoxyribonuclease, endoglucanase, esterase, glucoamylase, invertase, laccase, lipase, mannosidase, mutanase, oxidase, pectinolytic enzyme, peroxidase, phytase, polyphenoloxidase, proteolytic enzyme, ribonuclease, transglutaminase, or xylanase. An embodiment is a cleaning or detergent composition comprising a beta-glucanase polypeptide of the invention (e.g. a variant of the present invention) and one or more amylases.

The compositions may be prepared in accordance with methods known in the art and may be in the form of a liquid or a dry composition. The compositions may be stabilized in accordance with methods known in the art. An embodiment is a cleaning or detergent composition comprising a beta-glucanase polypeptide of the invention (e.g. a variant of the present invention) and one or more amylases.

Examples are given below of preferred uses of the compositions of the present invention. The dosage of the composition and other conditions under which the composition is used may be determined on the basis of methods known in the art.

### Uses

The beta-glucanases of the invention (e.g. a variant of the present invention) or the compositions of the invention may be used in applications where beta-glucan (e.g. beta-D-glucan, beta-1,3-1,4 glucan, mix-linkage beta-glucan, barley beta-glucan, oatmeal beta-glucan) needs to be degraded (e.g. under alkaline conditions and/or in the presence of an oxidizing agent (e.g. a bleaching agent)). Accordingly, the present invention relates to uses of a variant comprising a substitution at one or more positions corresponding to positions 33 and 188 of the mature polypeptide of SEQ ID NO: 26 using the numbering of SEQ ID NO: 26, wherein the variant has beta-glucanase activity and wherein the variant has at least 60%, e.g., at least 65%, at least 70%, at least 75%, at least 80%, at least 85%, at least 90%, at least 95%, at least 95.5%, at least 96%, at least 96.5%, at least 97%, at least 97.5%, at least 98%, at least 98.5%, at least 99%, or at least 99.5%, but less than 100% sequence identity to the mature polypeptide of any of: SEQ ID NO: 26, SEQ ID NO: 27, SEQ ID NO: 25, and SEQ ID NO: 28, or a composition comprising a variant comprising a substitution at one or more positions corresponding to positions 33 and 188 of the mature polypeptide of SEQ ID NO: 26 using the numbering of SEQ ID NO: 26, wherein the variant has beta-glucanase activity and wherein the variant has at least 60%, e.g., at least 65%, at least 70%, at least 75%, at least 80%, at least 85%, at least 90%, at least 95%, at least 95.5%, at least 96%, at least 96.5%, at least 97%, at least 97.5%, at least 98%, at least 98.5%, at least 99%, or at least 99.5%, but less than 100% sequence identity to the mature polypeptide of any of: SEQ ID NO: 26, SEQ ID NO: 27, SEQ ID NO: 25, and SEQ ID NO: 28, wherein the use or composition to be used, is for degrading a beta-glucan, preferably a beta-D-glucan, such as a beta-1,3-1,4 glucan, optionally, is the use carried out under alkaline conditions having a pH of 7.5 (or above) and/or in the presence of an oxidizing agent (e.g. a bleaching agent).

In one embodiment a variant of the invention or a composition of the invention may be used for degrading a beta-glucan, preferably said beta-glucan is a beta-D-glucan, further preferably said beta-glucan is a beta-1,3-1,4 glucan, most preferably said beta-glucan is a mix-linkage beta-glucan, further most preferably said beta-glucan is a barley beta-glucan or oatmeal beta-glucan; optionally said use is carried out under alkaline conditions having pH 7.5 (or above) and/or in the presence of an oxidizing agent (e.g. a bleaching agent).

In one embodiment a variant of the invention or a composition of the invention may be used for washing or cleaning a textile and/or a hard surface such as dish wash including Automatic Dish Wash (ADW); optionally said use is carried out under alkaline conditions having pH 7.5 (or above) and/or in the presence of an oxidizing agent (e.g. a bleaching agent). An embodiment is a cleaning or detergent composition comprising a beta-glucanase polypeptide of the invention (e.g. a variant of the present invention) and one or more amylases. Examples of where beta-glucanases could be used include detergent applications, paper and pulp productions. In one aspect, beta-glucanases of the invention (e.g. a variant of the present invention) may be used for washing or cleaning a textile and/or a hard surface such as dish wash including Automatic Dish Wash (ADW), Hand Dish Wash (HDW), and/or in a cleaning process such as laundry or hard surface cleaning including dish wash including Automatic Dish Wash (ADW) and industrial cleaning, and/or for laundering and/or hard surface cleaning including dish wash including Automatic Dish Wash (ADW), and/or for at least one of the following: preventing, reducing or removing a biofilm and/or malodor from an item, and/or for anti-redeposition. An embodiment is a cleaning or detergent composition comprising a beta-glucanase polypeptide of the invention (e.g. a variant of the present invention) and one or more amylases.

Biofilm can develop on textile when microorganisms are present on an item and stick together on the item. Some microorganisms tend to adhere to the surface of items such as textiles. Some microorganisms adhere to such surfaces and form a biofilm on the surface. The biofilm may be sticky and the adhered microorganisms and/or the biofilm may be difficult to remove. Furthermore the biofilm adhere soil due to the sticky nature of the biofilm. The commercial laundry detergent compositions available on the marked do not remove such adhered microorganisms or biofilm.

The present invention concerns the use of a polypeptide having beta-glucanase activity (e.g. a variant of the present invention) for preventing, reducing or removing a biofilm from an item, wherein the polypeptide is obtained from a bacterial source and wherein the item is a textile. An embodiment is a cleaning or detergent composition comprising a beta-glucanase polypeptide of the invention (e.g. a variant of the present invention) and one or more amylases. In one embodiment of the invention the polypeptide having beta-glucanase activity (e.g. a variant of the present invention) is used for preventing, reducing or removing the stickiness of an item. An embodiment is a cleaning or detergent composition comprising a beta-glucanase polypeptide of the invention (e.g. a variant of the present invention) and one or more amylases.

### Compositions

The present invention also relates to compositions comprising a beta-glucanase of the invention (e.g., a polypeptide of the present invention, i.e. a variant of the present invention). Accordingly, the present invention relates to compositions comprising a variant comprising a substitution at one or more positions corresponding to positions 33 and 188 of the mature polypeptide of SEQ ID NO: 26 using the numbering of SEQ ID NO: 26, wherein the variant has beta-glucanase activity and wherein the variant has at least 60%, e.g., at least 65%, at least 70%, at least 75%, at least 80%, at least 85%, at least 90%, at least 95%, at least 95.5%, at least 96%, at least 96.5%, at least 97%, at least 97.5%, at least 98%, at least 98.5%, at least 99%, or at least 99.5%, but less than 100% sequence identity to the mature polypeptide of any of: SEQ ID NO: 26, SEQ ID NO: 27, SEQ ID NO: 25, and SEQ ID NO: 28.

The present invention also relates to compositions comprising a beta-glucanase of the invention and one or more additional enzymes. The present invention also relates to compositions comprising a beta-glucanase of the invention and one or more amylases, preferably said one or more amylases is one or more alpha-amylases. An embodiment is a cleaning or detergent composition comprising a beta-glucanase polypeptide of the invention (e.g. a variant of the present invention) and one or more amylases.

In one embodiment, the present invention relates to compositions in particular to cleaning compositions and/or detergent compositions comprising a beta-glucanase of the invention (e.g. a variant of the present invention) and a suitable surfactant. An embodiment is a cleaning or detergent composition comprising a beta-glucanase polypeptide of the invention (e.g. a variant of the present invention) and one or more amylases.

In one embodiment, the present invention relates to compositions in particular to cleaning compositions and/or detergent compositions comprising a beta-glucanase of the invention (e.g. a variant of the present invention) and a bleaching agent. An embodiment is a cleaning or detergent composition comprising a beta-glucanase polypeptide of the invention (e.g. a variant of the present invention) and one or more amylases.

In one embodiment, the detergent composition may be adapted for specific uses such as laundry, in particular household laundry, dish washing or hard surface cleaning.

In another embodiment a composition of the present invention is a cleaning or a detergent composition.

In one embodiment a cleaning or detergent composition of the invention comprises a variant of the invention and one or more amylases such that said variant and said one or more amylases have a synergistic effect; preferably said synergistic effect is a REM synergistic effect, further preferably said REM synergistic effect is of more than 6.5 at about 40° C. for about 30 minutes at pH of about 7.5, further preferably said REM synergistic effect is of more than 6.1 at about 40° C. for about 30 minutes at pH of about 10, further preferably said REM synergistic effect is of more than 6.2 at about 40° C. for about 30 minutes at pH of about 10.

In one embodiment a cleaning or detergent composition of the invention comprises a variant of the invention and one or more amylases such that said variant is capable of having beta-glucanase activity in an aqueous solution with a pH in the range from about 7.5 to about 13.5, wherein said aqueous solution optionally comprises a bleaching agent, preferably said pH is in the range from about 7.5 to about 12.5, further preferably said pH is in the range from about 8.5 to about 11.5, most preferably said pH is in the range from about 9.5 to about 10.5.

In one embodiment a cleaning or detergent composition of the invention comprises a variant of the invention and one or more amylases such that said variant is capable of showing beta-glucanase activity in an aqueous solution at a temperature selected in the range from about 20° C. to about 75° C., and/or in the range from about 40° C. to about 60° C., wherein said aqueous solution optionally comprises a bleaching agent.

In one embodiment a cleaning or detergent composition of the invention comprises a variant of the invention and one or more amylases such that said variant is capable of having beta-glucanase activity for at least 15 minutes, preferably for at least 30 minutes, further preferably for at least 60 minutes, further most preferably for at least 90 minutes, further most preferably for at least 120 minutes.

In one embodiment a cleaning or detergent composition of the invention comprises a variant of the invention and one or more amylases such that the beta-glucanase activity of the variant comprises alkaline beta-glucanase activity, wherein said alkaline beta-glucanase activity is beta-glucanase activity at pH 7.5 or above.

In one embodiment a cleaning or detergent composition of the invention comprises a variant of the invention and one or more amylases such that the beta-glucanase activity of the variant comprises licheninase EC 3.2.1.73 activity, preferably said beta-glucanase activity is licheninase EC 3.2.1.73 activity.

In one embodiment a cleaning or detergent composition of the invention comprises a variant of the invention and one or more amylases such that said amylase is an alpha-amylase.

In one embodiment a cleaning or detergent composition of the invention comprises a variant of the invention and one or more amylases and further comprises one or more detergent components.

In one embodiment the detergent component is selected from the group consisting of: surfactants, hydrotropes, builders, co-builders, chelators, bleach components, polymers, fabric hueing agents, fabric conditioners, foam boosters, suds suppressors, dispersants, dye transfer inhibitors, fluorescent whitening agents, perfume, optical brighteners, bactericides, fungicides, soil suspending agents, soil release polymers, anti-redeposition agents, enzyme inhibitors, enzyme stabilizers, enzyme activators, antioxidants, and solubilizers.

In one embodiment a cleaning or detergent composition of the invention comprises a variant of the invention and one or more amylases and further comprises one or more additional enzymes.

In one embodiment a cleaning or detergent composition of the invention comprises a variant of the invention and one or more amylases and further comprises an enzyme selected from the group consisting of: DNases, perhydrolases, amylases, proteases, peroxidases, cellulases, betaglucanases, xyloglucanases, hemicellulases, xanthanases, xanthan lyases, lipases, acyl transferases, phospholipases, esterases, laccases, catalases, aryl esterases, amylases, alpha-amylases, glucoamylases, cutinases, pectinases, pectate lyases, keratinases, reductases, oxidases, phenoloxidases, lipoxygenases, ligninases, carrageenases, pullulanases, tannases, arabinosidases, hyaluronidases, chondroitinases, xyloglucanases, xylanases, pectin acetyl esterases, polygalacturonases, rhamnogalacturonases, other endo-beta-mannanases, exo-beta-mannanases, pectin methylesterases, cellobiohydrolases, transglutaminases, and combinations thereof.

In one embodiment a cleaning or detergent composition of the invention comprises a variant of the invention and one or more amylases such that said composition has pH of 7.5 or above and optionally, comprises a bleaching agent; preferably said pH is selected in the range from about 7.5 to about 13.5, further preferably said pH is selected in the range from about 7.5 to about 12.5, most preferably said pH is selected in the range from about 8.5 to about 11.5, further most preferably said pH is selected in the range from about 9.5 to about 10.5.

In one embodiment a cleaning or detergent composition of the invention comprises a variant of the invention and one or more amylases such that said alpha-amylase is selected from the group consisting of:

(a) a polypeptide having at least 90% sequence identity to SEQ ID NO: 13 (corresponding to SEQ ID NO: 2 of WO 95/10603);

(b) a polypeptide having at least 90% sequence identity to SEQ ID NO: 13 (corresponding to SEQ ID NO: 2 in WO 95/10603), wherein the polypeptide comprises a substitution in one or more of positions: 15, 23, 105, 106, 124, 128, 133, 154, 156, 178, 179, 181, 188, 190, 197, 201, 202, 207, 208, 209, 211, 243, 264, 304, 305, 391, 408, and/or 444;

(c) a polypeptide having at least 90% sequence identity to SEQ ID NO: 14 (corresponding to SEQ ID NO: 6 in WO 02/010355);

(d) a polypeptide having at least 90% sequence identity to the hybrid polypeptide of SEQ ID NO: 15 (comprising residues 1-33 of SEQ ID NO: 6 of WO 2006/066594 and residues 36-483 of SEQ ID NO: 4 of WO 2006/066594);

(e) a polypeptide having at least 90% sequence identity to the hybrid polypeptide of SEQ ID NO: 15 (comprising residues 1-33 of SEQ ID NO: 6 of WO 2006/066594 and residues 36-483 of SEQ ID NO: 4 of WO 2006/066594), wherein the hybrid polypeptide comprises a substitution, a deletion or an insertion in one of more of positions: 48, 49, 107, 156, 181, 190, 197, 201, 209 and/or 264;

(f) a polypeptide having at least 90% sequence identity to SEQ ID NO: 16 (corresponding to SEQ ID NO: 6 of WO 02/019467);

(g) a polypeptide having at least 90% sequence identity to SEQ ID NO: 16 (corresponding to SEQ ID NO: 6 of WO 02/019467), wherein the polypeptide comprises a substitution, a deletion or an insertion in one of more of positions: 181, 182, 183, 184, 195, 206, 212, 216 and/or 269;

(h) a polypeptide having at least 90% sequence identity to SEQ ID NO: 17, SEQ ID NO: 18 or SEQ ID NO: 19 (corresponding to SEQ ID NO: 1, SEQ ID NO: 2 or SEQ ID NO: 7 of WO 96/023873)

(i) a polypeptide having at least 90% sequence identity to SEQ ID NO: 17, SEQ ID NO: 18 or SEQ ID NO: 19 (corresponding to SEQ ID NO: 1, SEQ ID NO: 2 or SEQ ID NO: 7 of WO 96/023873), wherein the polypeptide comprises a substitution, a deletion or an insertion in one of more of positions: 140, 183, 184 195, 206, 243, 260, 304 and/or 476;

(j) a polypeptide having at least 90% sequence identity to SEQ ID NO: 20 (corresponding to SEQ ID NO: 2 of WO 08/153815);

(k) a polypeptide having at least 90% sequence identity to SEQ ID NO: 21 (corresponding to SEQ ID NO: 10 of WO 01/66712);

(l) a polypeptide having at least 90% sequence identity to SEQ ID NO: 21 (corresponding to SEQ ID NO: 10 of WO 01/66712), wherein the polypeptide comprises a substitution, a deletion or an insertion in one of more of positions: 176, 177, 178, 179, 190, 201, 207, 211 and/or 264;

(m) a polypeptide having at least 90% sequence identity to SEQ ID NO: 22 (corresponding to SEQ ID NO: 2 of WO 09/061380);

(n) a polypeptide having at least 90% sequence identity to SEQ ID NO: 22 (corresponding to SEQ ID NO: 2 of WO 09/061380), wherein the polypeptide comprises a substitution, a deletion or an insertion in one of more of positions: 87, 98, 125, 128, 131, 165, 178, 180, 181, 182, 183, 201, 202, 225, 243, 272, 282, 305, 309, 319, 320, 359, 444 and/or 475;

(o) a polypeptide having at least 90% sequence identity to SEQ ID NO: 21, wherein the polypeptide comprises a substitution, a deletion or an insertion in one of more of positions: 28, 118, 174; 181, 182, 183, 184, 186, 189, 195, 202, 298, 299, 302, 303, 306, 310, 314; 320, 324, 345, 396, 400, 439, 444, 445, 446, 449, 458, 471 and/or 484;

(p) a polypeptide having at least 90% sequence identity to SEQ ID NO: 12;

(r) a polypeptide having at least 90% sequence identity to SEQ ID NO: 12 (corresponding to SEQ ID NO: 2 in WO 95/10603), wherein the polypeptide comprises a substitution in one or more of positions: 15, 23, 105, 106, 124, 128, 133, 154, 156, 178, 179, 181, 188, 190, 197, 201, 202, 207, 208, 209, 211, 243, 264, 304, 305, 391, 408, and/or 444;

(s) a polypeptide having at least 90% sequence identity to SEQ ID NO: 29; and

(t) a polypeptide having at least 90% sequence identity to SEQ ID NO: 29, wherein the polypeptide comprises a substitution in one or more of positions: 187, 203, 476, 458, 459, 460, 178, 179, 180, 181, 7, 200, 126, 132, 303, 477, 15, 23, 105, 106, 124, 128, 133, 154, 156, 178, 179, 181, 188, 190, 197, 201, 202, 207, 208, 209, 211, 243, 264, 304, 305, 391, 408, and/or 444.

In one embodiment a cleaning or detergent composition of the invention comprises a variant of the invention and one or more amylases such that said composition has improved stability and/or performance under alkaline conditions, preferably said alkaline conditions have pH 7.5 or above.

In one embodiment a cleaning or detergent composition of the invention comprises a variant of the invention and one or more amylases such that said composition is in form selected from a group consisting of: a bar, a homogenous tablet, a tablet having two or more layers, a pouch having one or more compartments, a regular or compact powder, a granule, a paste, a gel, or a regular, compact or concentrated liquid.

In one embodiment a cleaning or detergent composition of the invention comprises a variant of the invention and one or more amylases such that said cleaning or detergent composition has an enzyme detergency benefit in cleaning or detergent applications.

In one embodiment a cleaning or detergent composition of the invention comprises a variant of the invention and one or more amylases such that said cleaning or detergent composition has improved stability and/or performance, preferably said improved stability and/or performance is under alkaline conditions having pH 7.5 (or above) and/or in the presence of an oxidizing agent (e.g. a bleaching agent).

In one embodiment the present invention relates to a method for removing a stain from a surface which comprises contacting the surface with a composition of the invention.

Alkaline liquid detergents having high pH are widely used in cleaning, such as laundry and dish wash cleaning. Liquid detergents with elevated pH are especially commonly used by consumers in North America. The high pH cleaning compositions are also used in industrial cleaning processes. Alkaline detergents include liquids having detergent properties. The pH of such detergents usually ranges in pH from 9 to 12.5. The high pH detergents typically comprise components such as surfactants, builders and bleach components and additionally they may also contain a significant amount of water and alkalis such as NaOH, TSP (Trisodium phosphate), ammonia, Sodium carbonate, Potassium hydroxide (KOH) these alkalis are usually added in amount corresponding to 0.1 to 30 percent weight (wt). Adding enzymes to detergents are highly advantageous as the specific activities of these enzymes effectively removes specific stains from surfaces such as textile and cutlery. However, the difficulty of maintaining acceptable enzyme stability in the high pH liquid detergents has for many years prohibited inclusion of enzymes into these detergents. In another embodiment the present invention relates high pH liquid cleaning compositions comprising an alkaline stable beta-glucanase of the present invention (e.g. a variant of the present invention) suitable for use in such compositions.

In another embodiment a composition of the present invention preferably contains alkaline buffer system to provide a pH of at least about 7.5, at least about 8, at least about 9, preferably pH 10 or above. Preferably the pH is from about 9 to about 13. In order to achieve the high pH it is necessary to have present an alkali metal hydroxide especially sodium or potassium hydroxide, normally in an amount of 0.1 to about 30% by weight (percentage by weight, abbreviated wt %) of the composition, and preferably 1.0 to 2.5%, or higher amounts of a suitable alkali metal silicate such as metal silicate, according to the desired pH for the product.

In another embodiment a composition of the present invention has pH of 7.5 or above and optionally comprises a bleaching agent; preferably said pH is selected in the range from about 7.5 to about 13.5, further preferably said pH is selected in the range from about 7.5 to about 12.5, most preferably said pH is selected in the range from about 8.5 to about 11.5, further most preferably said pH is selected in the range from about 9.5 to about 10.5.

The detergent compositions of the invention may be formulated, for example, as a hand or machine laundry detergent composition including a laundry additive composition suitable for pre-treatment of stained fabrics and a rinse added fabric softener composition, or be formulated as a detergent composition for use in general household hard surface cleaning operations, or be formulated for hand or machine dishwashing operations. The detergent compositions of the invention may find use in hard surface cleaning, automatic dishwashing applications, as well as cosmetic applications such as dentures, teeth, hair and skin. An embodiment is a cleaning or detergent composition comprising a beta-glucanase polypeptide of the invention (e.g. a variant of the present invention) and one or more amylases.

The detergent composition of the invention may be in any convenient form, e.g., a bar, a tablet, a powder, a granule, a paste or a liquid. A liquid detergent may be aqueous, typically containing up to 70% water and 0-30% organic solvent, or non-aqueous. An embodiment is a cleaning or detergent composition comprising a beta-glucanase polypeptide of the invention (e.g. a variant of the present invention) and one or more amylases.

Unless otherwise noted, all component or composition levels provided herein are made in reference to the active level of that component or composition, and are exclusive of impurities, for example, residual solvents or by-products, which may be present in commercially available sources. An embodiment is a cleaning or detergent composition comprising a beta-glucanase polypeptide of the invention (e.g. a variant of the present invention) and one or more amylases.

The beta-glucanase of the invention is normally incorporated in the detergent composition at a level of from 0.000001% to 2% of enzyme protein by weight of the composition, preferably at a level of from 0.00001% to 1% of enzyme protein by weight of the composition, more preferably at a level of from 0.0001% to 0.75% of enzyme protein by weight of the composition, even more preferably at a level of from 0.001% to 0.5% of enzyme protein by weight of the composition. An embodiment is a cleaning or detergent composition comprising a beta-glucanase polypeptide of the invention (e.g. a variant of the present invention) and one or more amylases.

Furthermore, the beta-glucanase of the invention is normally incorporated in the detergent composition in such amounts that their concentration in the wash water is at a level of from 0.0000001% to 1% enzyme protein, preferably at a level of from 0.000005% to 0.01% of enzyme protein, more preferably at a level of from 0.000001% to 0.005% of enzyme protein, even more preferably at a level of from 0.00001% to 0.001% of enzyme protein in wash water. An embodiment is a cleaning or detergent composition comprising a beta-glucanase polypeptide of the invention (e.g. a variant of the present invention) and one or more amylases.

As is well known, the amount of enzyme will also vary according to the particular application and/or as a result of the other components included in the compositions.

A composition for use in automatic dishwash (ADW), for example, may include 0.001%-50%, such as 0.01%-25%, such as 0.02%-20%, such as 0.1-15% of enzyme protein by weight of the composition. An embodiment is a cleaning or detergent composition comprising a beta-glucanase polypeptide of the invention and one or more amylases.

A composition for use in laundry granulation, for example, may include 0.0001%-50%, such as 0.001%-20%, such as 0.01%-15%, such as 0.05%-10% of enzyme protein by weight of the composition. An embodiment is a cleaning or detergent composition comprising a beta-glucanase polypeptide of the invention (e.g. a variant of the present invention) and one or more amylases.

A composition for use in laundry liquid, for example, may include 0.0001%-10%, such as 0.001-7%, such as 0.1%-5% of enzyme protein by weight of the composition. An embodiment is a cleaning or detergent composition comprising a beta-glucanase polypeptide of the invention (e.g. a variant of the present invention) and one or more amylases.

In some preferred embodiments, the detergent compositions provided herein are typically formulated such that, during use in aqueous cleaning operations, the wash water has a pH of from about 5.0 to about 13.5, or in alternative embodiments, even from about 6.0 to about 10.5, such as from about 5 to about 11, from about 5 to about 10, from about 5 to about 9, from about 5 to about 8, from about 5 to about 7, from about 6 to about 11, from about 6 to about 10, from about 6 to about 9, from about 6 to about 8, from about 6 to about 7, from about 7 to about 11, from about 7 to about 10, from about 7 to about 9, or from about 7 to about 8. Preferably, the detergent compositions provided herein are typically formulated such that, during use in aqueous cleaning operations, the wash water has a pH selected in the range from about 7.5 to about 13.5, further preferably said pH is selected in the range from about 8.5 to about 11.5, most preferably said pH is selected in the range from about 9.5 to about 10.5; further most preferably pH 7.5 or above. An embodiment is a cleaning or detergent composition comprising a beta-glucanase polypeptide of the invention (e.g. a variant of the present invention) and one or more amylases.

In one embodiment, the beta-glucanase of the invention (e.g. a variant of the present invention) has improved stability, in particular improved storage stability in a high pH liquid cleaning composition, compared to known beta-glucanases. In a preferred embodiment, the beta-glucanase of the invention has improved stability, in particular improved storage stability, and on par or improved wash performance compared to the known beta-glucanases. An embodiment is a cleaning or detergent composition comprising a beta-glucanase polypeptide of the invention (e.g. a variant of the present invention) and one or more amylases.

In one embodiment, the beta-glucanase of the invention (e.g. a variant of the present invention) has an improved property relative to the parent, wherein the improved property is increased oxidation stability. An embodiment is a cleaning or detergent composition comprising a beta-glucanase polypeptide of the invention (e.g. a variant of the present invention) and one or more amylases.

In some preferred embodiments, granular or liquid laundry products are formulated such that the wash water has a pH from about 5.5 to about 8. In other preferred embodiments, granular or liquid laundry products are formulated such that the wash water has a pH selected in the range from about 7.5 to about 13.5, further preferably said pH is selected in the range from about 8.5 to about 11.5, most preferably said pH is selected in the range from about 9.5 to about 10.5; further most preferably pH 7.5 or above. Techniques for controlling pH at recommended usage levels include the use of buffers, alkalis, acids, etc., and are well known to those skilled in the art. An embodiment is a cleaning or detergent composition comprising a beta-glucanase polypeptide of the invention (e.g. a variant of the present invention) and one or more amylases.

Enzyme components weights are based on total protein. All percentages and ratios are calculated by weight unless otherwise indicated. All percentages and ratios are calculated based on the total composition unless otherwise indicated. In the exemplified detergent composition, the enzymes levels are expressed by pure enzyme by weight of the total composition and unless otherwise specified, the detergent ingredients are expressed by weight of the total composition.

The enzymes of the present invention also find use in detergent additive products. A detergent additive product comprising a beta-glucanase of the invention (e.g. a variant of the present invention) is suited for inclusion in a wash process when, e.g., temperature is low, such as at temperatures about 40° C. or below, the pH is between 6 and 8 and the washing time short, e.g., below 30 min. A detergent additive product comprising a beta-glucanase of the invention (e.g. a variant of the present invention) is further ideally suited for inclusion in a alkaline wash process when, e.g., a pH selected in the range from about 7.5 to about 13.5, a temperature selected in the range from about 20° C. to about 75° C., and the washing time short, e.g., below 30 min, e.g. at least 15 minutes. An embodiment is a cleaning or detergent composition comprising a beta-glucanase polypeptide of the invention (e.g. a variant of the present invention) and one or more amylases.

The detergent additive product may be a beta-glucanase of the invention (e.g. a variant of the present invention) and preferably an additional enzyme. In one embodiment, the additive is packaged in dosage form for addition to a cleaning process. The single dosage may comprise a pill, tablet, gelcap or other single dosage unit including powders and/or liquids. In some embodiments, filler and/or carrier material(s) are included, suitable filler or carrier materials include, but are not limited to, various salts of sulfate, carbonate and silicate as well as talc, clay and the like. In some embodiments filler and/or carrier materials for liquid compositions include water and/or low molecular weight primary and secondary alcohols including polyols and diols. Examples of such alcohols include, but are not limited to, methanol, ethanol, propanol and isopropanol.

In one particularly preferred embodiment the beta-glucanase according to the invention (e.g. a variant of the present invention) is employed in a granular composition or liquid, the beta-glucanase may be in form of an encapsulated particle. In one embodiment, the encapsulating material is selected from the group consisting of carbohydrates, natural or synthetic gums, chitin and chitosan, cellulose and cellulose derivatives, silicates, phosphates, borates, polyvinyl alcohol, polyethylene glycol, paraffin waxes and combinations thereof.

The compositions according to the invention typically comprise one or more detergent ingredients. The term detergent compositions include articles and cleaning and treatment compositions. The term cleaning composition includes, unless otherwise indicated, tablet, granular or powder- form all-purpose or “heavy-duty” washing agents, especially laundry detergents; liquid, gel or paste-form all-purpose washing agents, especially the so-called heavy-duty liquid types; liquid fine-fabric detergents; hand dishwashing agents or light duty dishwashing agents, especially those of the high-foaming type; machine dishwashing agents, including the various tablet, granular, liquid and rinse-aid types for household and institutional use. The composition can also be in unit dose packages, including those known in the art and those that are water soluble, water insoluble and/or water permeable.

In embodiments in which cleaning and/or detergent components may not be compatible with the beta-glucanase of the present invention (e.g. a variant of the present invention), suitable methods may be used for keeping the cleaning and/or detergent components and the beta-glucanase separated (i.e., not in contact with each other) until combination of the two components is appropriate. Such separation methods include any suitable method known in the art (e.g., gelcaps, encapsulation, tablets, and physical separation e.g., by use of a water dissolvable pouch having one or more compartments).

As mentioned when the beta-glucanase of the invention (e.g. a variant of the present invention) is employed as a component of a detergent composition (e.g., a laundry washing detergent composition, or a dishwashing detergent composition), it may, for example, be included in the detergent composition in the form of a non-dusting granulate, a stabilized liquid, or a protected enzyme. Non-dusting granulates may be produced, e.g., as disclosed in U.S. Pat. Nos. 4,106,991 and 4,661,452 (both to Novo Industri A/S) and may optionally be coated by methods known in the art. Examples of waxy coating materials are polyethyleneglycol (PEG) products with mean molecular weights of 1000 to 20000; ethoxylated nonylphenols having from 16 to 50 ethylene oxide units; ethoxylated fatty alcohols in which the alcohol contains from 12 to 20 carbon atoms and in which there are 15 to 80 ethylene oxide units; fatty alcohols; fatty acids; and mono- and di- and triglycerides of fatty acids. Examples of film-forming coating materials suitable for application by fluid bed techniques are given in GB 1483591.

In some embodiments, the enzymes employed herein are stabilized by the presence of water-soluble sources of zinc (II), calcium (II) and/or magnesium (II) ions in the finished compositions that provide such ions to the enzymes, as well as other metal ions (e.g., barium (II), scandium (II), iron (II), manganese (II), aluminum (III), tin (II), cobalt (II), copper (II), nickel (II), and oxovanadium (IV)). The enzymes of the detergent compositions of the invention may also be stabilized using conventional stabilizing agents such as polyol, e.g., propylene glycol or glycerol, a sugar or sugar alcohol, lactic acid, and the composition may be formulated as described in, e.g., WO 92/19709 and WO 92/19708. The enzymes of the invention may also be stabilized by adding reversible enzyme inhibitors, e.g., of the protein type (as described in EP 544 777) or the boronic acid type. Other enzyme stabilizers are well known in the art, such as peptide aldehydes and protein hydrolysate, e.g. the beta-glucanase according to the invention may be stabilized using peptide aldehydes or ketones such as described in WO2005/105826 and WO2009/118375.

Protected enzymes for inclusion in a detergent composition of the invention may be prepared, as mentioned above, according to the method disclosed in EP 238 216.

The composition may be augmented with one or more agents for preventing or removing the formation of the biofilm. These agents may include, but are not limited to, dispersants, surfactants, detergents, other enzymes, anti-microbials, and biocides.

The compositions of the invention may be applied in dosing elements to be used in an auto-dosing device. The dosing elements comprising the composition of the present invention can be placed into a delivery cartridge as that described in WO 2007/052004 and WO 2007/0833141. The dosing elements can have an elongated shape and set into an array forming a delivery cartridge which is the refill for an auto-dosing dispensing device as described in case WO 2007/051989. The delivery cartridge is to be placed in an auto-dosing delivery device, such as that described in WO 2008/053191.

Suitable disclosure of auto-dosing devices can be found in WO 2007/083139, WO 2007/051989, WO 2007/083141, WO 2007/083142 and EP2361964,

### Other Enzymes

In one embodiment, a beta-glucanase of the invention (e.g. a variant of the present invention) is combined with one or more enzymes, such as at least two enzymes, more preferred at least three, four or five enzymes. Preferably, the enzymes have different substrate specificity, e.g., proteolytic activity, amylolytic activity, lipolytic activity, hemicellulytic activity or pectolytic activity. An embodiment is a cleaning or detergent composition comprising a beta-glucanase polypeptide of the invention (e.g. a variant of the present invention) and one or more amylases.

The detergent additive as well as the detergent composition may comprise one or more enzymes such as a protease, lipase, cutinase, an amylase, carbohydrase, cellulase, pectinase, mannanase, arabinase, galactanase, xylanase, oxidase, e.g., a laccase and/or peroxidase.

In general the properties of the selected enzyme(s) should be compatible with the selected detergent, (i.e., pH-optimum, compatibility with other enzymatic and non-enzymatic ingredients, etc.), and the enzyme(s) should be present in effective amounts.

Cellulases: Suitable cellulases include those of animal, vegetable or microbial origin. Particularly suitable cellulases include those of bacterial or fungal origin. Chemically modified or protein engineered variants are included. Suitable cellulases include cellulases from the genera Bacillus, Pseudomonas, Humicola, Fusarium, Thielavia, Acremonium, e.g., the fungal cellulases produced from Humicola insolens, Myceliophthora thermophila and Fusarium oxysporum disclosed in U.S. Pat. Nos. 4,435,307, 5,648,263, 5,691,178, 5,776,757 and WO 89/09259.

Especially suitable cellulases are the alkaline or neutral cellulases having color care benefits. Examples of such cellulases are cellulases described in EP 0 495 257, EP 0 531 372, WO 96/11262, WO 96/29397, WO 98/08940. Other examples are cellulase variants such as those described in WO 94/07998, EP 0 531 315, U.S. Pat. Nos. 5,457,046, 5,686,593, 5,763,254, WO 95/24471, WO 98/12307 and WO 1999/001544.

Commercially available cellulases include Celluzyme®, and Carezyme® (Novozymes A/S), Clazinase®, and Puradax HA® (Genencor International Inc.), and KAC-500(B)® (Kao Corporation).

Proteases: Suitable proteases include those of bacterial, fungal, plant, viral or animal origin e.g. microbial or vegetable origin. Microbial origin is preferred. Chemically modified or protein engineered variants are included. It may be an alkaline protease, such as a serine protease or a metalloprotease. A serine protease may for example be of the 51 family, such as trypsin, or the S8 family such as subtilisin. A metalloproteases protease may for example be a thermolysin from e.g. family M4 or other metalloprotease such as those from M5, M7 or M8 families.

The term “subtilases” refers to a sub-group of serine protease according to Siezen et al., Protein Engng. 4 (1991) 719-737 and Siezen et al. Protein Science 6 (1997) 501-523. Serine proteases are a subgroup of proteases characterized by having a serine in the active site, which forms a covalent adduct with the substrate. The subtilases may be divided into 6 sub-divisions, i.e. the Subtilisin family, the Thermitase family, the Proteinase K family, the Lantibiotic peptidase family, the Kexin family and the Pyrolysin family.

Examples of subtilases are those derived from Bacillus such as Bacillus lentus, B. alkalophilus, B. subtilis, B. amyloliquefaciens, Bacillus pumilus and Bacillus gibsonii described in; U.S. Pat. No. 7,262,042 and WO09/021867, and subtilisin lentus, subtilisin novo, subtilisin Carlsberg, Bacillus licheniformis, subtilisin BPN', subtilisin 309, subtilisin 147 and subtilisin 168 described in WO89/06279 and protease PD138 described in (WO93/18140). Other useful proteases may be those described in WO92/175177, WO01/016285, WO02/026024 and WO02/016547. Examples of trypsin-like proteases are trypsin (e.g. of porcine or bovine origin) and the Fusarium protease described in WO89/06270, WO94/25583 and WO05/040372, and the chymotrypsin proteases derived from Cellulomonas described in WO05/052161 and WO05/052146.

A further preferred protease is the alkaline protease from Bacillus lentus DSM 5483, as described for example in WO95/23221, and variants thereof which are described in WO92/21760, WO95/23221, EP1921147 and EP1921148.

Examples of metalloproteases are the neutral metalloprotease as described in WO07/044993 (Genencor Int.) such as those derived from Bacillus amyloliquefaciens.

Examples of useful proteases are the variants described in: WO92/19729, WO96/034946, WO98/20115, WO98/20116, WO99/011768, WO01/44452, WO03/006602, WO04/03186, WO04/041979, WO07/006305, WO11/036263, WO11/036264, especially the variants with substitutions in one or more of the following positions: 3, 4, 9, 15, 27, 36, 57, 68, 76, 87, 95, 96, 97, 98, 99, 100, 101, 102, 103, 104, 106, 118, 120, 123, 128, 129, 130, 160, 167, 170, 194, 195, 199, 205, 206, 217, 218, 222, 224, 232, 235, 236, 245, 248, 252 and 274 using the BPN′ numbering. More preferred the protease variants may comprise the mutations: S3T, V4I, S9R, A15T, K27R, *36D, V68A, N76D, N87S,R, *97E, A98S, S99G,D,A, S99AD, S101G,M,R S103A, V104I,Y,N, S106A, G118V,R, H120D,N, N123S, S128L, P129Q, S130A, G160D, Y167A, R170S, A194P, G195E, V199M, V205I, L217D, N218D, M222S, A232V, K235L, Q236H, Q245R, N252K, T274A (using BPN′ numbering).

Suitable commercially available protease enzymes include those sold under the trade names Alcalase®, Duralase™, Durazym™, Relase®, Relase® Ultra, Savinase®, Savinase® Ultra, Primase®, Polarzyme®, Kannase®, Liquanase®, Liquanase® Ultra, Ovozyme®, Coronase®, Coronase® Ultra, Neutrase®, Everlase® and Esperase® (Novozymes A/S), those sold under the tradename Maxatase®, Maxacal®, Maxapem®, Purafect®, Purafect Prime®, Preferenz™, Purafect MA®, Purafect Ox®, Purafect OxP®, Puramax®, Properase®, Effectenz™, FN2®, FN3®, FN4®, FN5®, FN6®, Excellase®, Opticlean® and Optimase® (Danisco/DuPont), Axapem™ (Gist-Brocases N.V.), BLAP (sequence shown in FIG. 29 of U.S. Pat. No. 5,352,604) and variants hereof (Henkel AG) and KAP (Bacillus alkalophilus subtilisin) from Kao.

Lipases: Suitable lipases include those of animal, vegetable or microbial origin. Particularly suitable lipases include those of bacterial or fungal origin. Chemically modified or protein engineered variants are included. Examples of useful lipases include lipases from Humicola (synonym Thermomyces), e.g., from H. lanuginosa (T. lanuginosus) as described in EP 258 068 and EP 305 216 or from H. insolens as described in WO 96/13580, a Pseudomonas lipase, e.g., from P. alcaligenes or P. pseudoalcaligenes (EP 218 272), P. cepacia (EP 331 376), P. stutzeri (GB 1,372,034), P. fluorescens, Pseudomonas sp. strain SD 705 (WO 95/06720 and WO 96/27002), P. wisconsinensis (WO 96/12012), a Bacillus lipase, e.g., from B. subtilis (Dartois et al., 1993, Biochemica et Biophysica Acta, 1131: 253-360), B. stearothermophilus (JP 64/744992) or B. pumilus (WO 91/16422).

Other examples are lipase variants such as those described in WO 92/05249, WO 94/01541, EP 407 225, EP 260 105, WO 95/35381, WO 96/00292, WO 95/30744, WO 94/25578, WO 95/14783, WO 95/22615, WO 97/04079 and WO 97/07202.

Preferred commercially available lipase enzymes include Lipolase™, Lipolase Ultra™, and Lipex™ (Novozymes A/S).

Amylases: Suitable amylases which can be used together with beta-glucanase of the invention (e.g. a variant of the present invention) may be an alpha-amylase or a glucoamylase and may be of bacterial or fungal origin. Chemically modified or protein engineered variants are included. Amylases include, for example, alpha-amylases obtained from Bacillus, e.g., a special strain of Bacillus licheniformis, described in more detail in GB 1,296,839. Suitable amylases include amylases having SEQ ID NO: 3 in WO 95/10603 or variants having 90% sequence identity to SEQ ID NO: 3 thereof. Preferred variants are described in WO 94/02597, WO 94/18314, WO 97/43424 and SEQ ID NO: 4 of WO 99/019467, such as variants with substitutions in one or more of the following positions: 15, 23, 105, 106, 124, 128, 133, 154, 156, 178, 179, 181, 188, 190, 197, 201, 202, 207, 208, 209, 211, 243, 264, 304, 305, 391, 408, and 444. Different suitable amylases include amylases having SEQ ID NO: 6 in WO 02/010355 or variants thereof having 90% sequence identity to SEQ ID NO: 6. Preferred variants of SEQ ID NO: 6 are those having a deletion in positions 181 and 182 and a substitution in position 193. Other amylases which are suitable are hybrid alpha-amylase comprising residues 1-33 of the alpha-amylase derived from B. amyloliquefaciens shown in SEQ ID NO: 6 of WO 2006/066594 and residues 36-483 of the B. licheniformis alpha-amylase shown in SEQ ID NO: 4 of WO 2006/066594 or variants having 90% sequence identity thereof. Preferred variants of this hybrid alpha-amylase are those having a substitution, a deletion or an insertion in one of more of the following positions: G48, T49, G107, H156, A181, N190, M197, I201, A209 and Q264. Most preferred variants of the hybrid alpha-amylase comprising residues 1-33 of the alpha-amylase derived from B. amyloliquefaciens shown in SEQ ID NO: 6 of WO 2006/066594 and residues 36-483 of SEQ ID NO: 4 are those having the substitutions:

M197T;

H156Y+A181T+N190F+A209V+Q264S; or

G48A+T49I+G107A+H156Y+A181T+N190F+I201F+A209V+Q2645.

Further amylases which are suitable are amylases having SEQ ID NO: 6 in WO 99/019467 or variants thereof having 90% sequence identity to SEQ ID NO: 6. Preferred variants of SEQ ID NO: 6 are those having a substitution, a deletion or an insertion in one or more of the following positions: R181, G182, H183, G184, N195, 1206, E212, E216 and K269. Particularly preferred amylases are those having deletion in positions R181 and G182, or positions H183 and G184. Additional amylases which can be used are those having SEQ ID NO: 1, SEQ ID NO: 3, SEQ ID NO: 2 or SEQ ID NO: 7 of WO 96/023873 or variants thereof having 90% sequence identity to SEQ ID NO: 1, SEQ ID NO: 2, SEQ ID NO: 3 or SEQ ID NO: 7. Preferred variants of SEQ ID NO: 1, SEQ ID NO: 2, SEQ ID NO: 3 or SEQ ID NO: 7 are those having a substitution, a deletion or an insertion in one or more of the following positions: 140, 181, 182, 183, 184, 195, 206, 212, 243, 260, 269, 304 and 476. More preferred variants are those having a deletion in positions 181 and 182 or positions 183 and 184. Most preferred amylase variants of SEQ ID NO: 1, SEQ ID NO: 2 or SEQ ID NO: 7 are those having a deletion in positions 183 and 184 and a substitution in one or more of positions 140, 195, 206, 243, 260, 304 and 476. Other amylases which can be used are amylases having SEQ ID NO: 2 of WO 08/153815, SEQ ID NO: 10 in WO 01/66712 or variants thereof having 90% sequence identity to SEQ ID NO: 2 of WO 08/153815 or 90% sequence identity to SEQ ID NO: 10 in WO 01/66712. Preferred variants of SEQ ID NO: 10 in WO 01/66712 are those having a substitution, a deletion or an insertion in one of more of the following positions: 176, 177, 178, 179, 190, 201, 207, 211 and 264. Further suitable amylases are amylases having SEQ ID NO: 2 of WO 09/061380 or variants having 90% sequence identity to SEQ ID NO: 2 thereof. Preferred variants of SEQ ID NO: 2 are those having a truncation of the C-terminus and/or a substitution, a deletion or an insertion in one of more of the following positions: Q87, Q98, S125, N128, T131, T165, K178, R180, S181, T182, G183, M201, F202, N225, S243, N272, N282, Y305, R309, D319, Q320, Q359, K444 and G475. More preferred variants of SEQ ID NO: 2 are those having the substitution in one of more of the following positions: Q87E,R, Q98R, S125A, N128C, T131I, T165I, K178L, T182G, M201L, F202Y, N225E,R, N272E,R, S243Q,A,E,D, Y305R, R309A, Q320R, Q359E, K444E and G475K and/or deletion in position R180 and/or S181 or of T182 and/or G183. Most preferred amylase variants of SEQ ID NO: 2 are those having the substitutions:

R180*+S181*+S243Q+G475K

N128C+K178L+T182G+Y305R+G475K;

N128C+K178L+T182G+F202Y+Y305R+D319T+G475K;

S125A+N128C+K178L+T182G+Y305R+G475K; or

S125A+N128C+T131I+T165I+K178L+T182G+Y305R+G475K wherein the variants are C-terminally truncated and optionally further comprises a substitution at position 243 and/or a deletion at position 180 and/or position 181.

Further suitable amylases are amylases having SEQ ID NO: 1 of WO13184577 or variants having 90% sequence identity to SEQ ID NO: 1 thereof. Preferred variants of SEQ ID NO: 1 are those having a substitution, a deletion or an insertion in one of more of the following positions: N126, E132, K176, R178, G179, T180, G181, E187, N192, M199, I203, S241, Y303 R458, T459, D460, G476 and G477. More preferred variants of SEQ ID NO: 1 are those having the substitution in one of more of the following positions: N126Y, E132HY, K176L, E187P, N192FYH, M199L, I203YF, S241QADN, Y303DN, R458N, T459S, D460T, G476K and G477K and/or deletion in position R178 and/or S179 or of T180 and/or G181. Most preferred amylase variants of SEQ ID NO: 1 are those having the substitutions:

E187P+I203Y+G476K

E187P+I203Y+R458N+T459S+D460T+G476K

N126Y+T180D+E187P+I203Y+Y303D+G476T

N126Y+E132H+T180D+E187P+I203Y+Y303D+G476T+G477E

N126Y+F153W+T180H+I203Y+S239Q

wherein the variants optionally further comprises a substitution at position 241 and/or a deletion at position 178 and/or position 179 or position 180 and/or position 181.

Other suitable amylases are the alpha-amylase having SEQ ID NO: 12 in WO01/66712 or a variant having at least 90% sequence identity to SEQ ID NO: 12. Preferred amylase variants are those having a substitution, a deletion or an insertion in one of more of the following positions of SEQ ID NO: 12 in WO01/66712: R28, R118, N174; R181, G182, D183, G184, G186, W189, N195, M202, Y298, N299, K302, S303, N306, R310, N314; R320, H324, E345, Y396, R400, W439, R444, N445, K446, Q449, R458, N471, N484. Particular preferred amylases include variants having a deletion of D183 and G184 and having the substitutions R118K, N195F, R320K and R458K, and a variant additionally having substitutions in one or more position selected from the group: M9, G149, G182, G186, M202, T257, Y295, N299, M323, E345 and A339, most preferred a variant that additionally has substitutions in all these positions. Other examples are amylase variants such as those described in WO2011/098531, WO2013/001078 and WO2013/001087. Commercially available amylases are Duramyl™, Termamyl™, Fungamyl™, Stainzyme ™, Stainzyme Plus™, Natalase™, Liquozyme X and BAN™ (from Novozymes A/S), and Rapidase™, Purastar™/Effectenz™, Powerase, Preferenz S1000, Preferenz S110 (R179*, G180*, E187P, I203Y, G476K, R458N, T459S, D460T), Preferenz S100 (R180*, S181*, S243Q, G475K) and Excellenz S2000 (from Genencor International Inc./DuPont). Especially suitable are oxidation stable amylases. Preferred amylases have other amino acids than methionine in the position corresponding to position M202 of SEQ ID NO: 12 in WO01/66712, e.g., an M202L substitutions. Examples of commercial oxidation stable amylases are Duramyl ™ and Stainzyme Plus™ (from Novozymes A/S) and Powerase and Excellenz S1000 (from Genencor International Inc./DuPont).

Other suitable amylases are variants disclosed in WO 2016/180748. In particular, variants of the amino acid sequence listed as SEQ ID NO: 13 or 14, wherein the variants comprises one or more modifications in the following positions: , 54, 56, 72, 109, 113, 116, 134, 140, 159, 167, 169, 172, 173, 174, 181, 182, 183, 184, 189, 194, 195, 206, 255, 260, 262, 265, 284, 289, 304, 305, 347, 391, 395, 439, 469, 444, 473, 476, or 477 wherein numbering is according to SEQ ID NO: 1 disclosed in WO 2016/180748, and wherein the variants have at least 75% sequence identity to SEQ ID NO: 13 or SEQ ID NO: 14 of WO 2016/180748.

Peroxidases/Oxidases: Suitable peroxidases/oxidases include those of plant, bacterial or fungal origin. Chemically modified or protein engineered variants are included. Examples of useful peroxidases include peroxidases from Coprinus, e.g., from C. cinereus, and variants thereof as those described in WO 93/24618, WO 95/10602, and WO 98/15257.

Commercially available peroxidases include Guardzyme® (Novozymes A/S).

The detergent enzyme(s) may be included in a detergent composition by adding separate additives containing one or more enzymes, or by adding a combined additive comprising all of these enzymes. A detergent additive of the invention, i.e., a separate additive or a combined additive, can be formulated, for example, as a granulate, liquid, slurry, etc. Preferred detergent additive formulations are granulates, in particular non-dusting granulates as described above, liquids, in particular stabilized liquids, or slurries.

Surfactants

Typically, the detergent composition comprises (by weight of the composition) one or more surfactants in the range of 0% to 50%, preferably from 2% to 40%, more preferably from 5% to 35%, more preferably from 7% to 30%, most preferably from 10% to 25%, even most preferably from 15% to 20%. In a preferred embodiment the detergent is a liquid or powder detergent comprising less than 40%, preferably less than 30%, more preferably less than 25%, even more preferably less than 20% by weight of surfactant. The composition may comprise from 1% to 15%, preferably from 2% to 12%, 3% to 10%, most preferably from 4% to 8%, even most preferably from 4% to 6% of one or more surfactants. Preferred surfactants are anionic surfactants, non-ionic surfactants, cationic surfactants, zwitterionic surfactants, amphoteric surfactants, and mixtures thereof. Preferably, the major part of the surfactant is anionic. Suitable anionic surfactants are well known in the art and may comprise fatty acid carboxylates (soap), branched-chain, linear-chain and random chain alkyl sulfates or fatty alcohol sulfates or primary alcohol sulfates or alkyl benzenesulfonates such as LAS and LAB or phenylalknesulfonates or alkenyl sulfonates or alkenyl benzenesulfonates or alkyl ethoxysulfates or fatty alcohol ether sulfates or alpha-olefin sulfonate or dodecenyl/tetradecnylsuccinic acid. The anionic surfactants may be alkoxylated. The detergent composition may also comprise from 1 wt % to 10 wt % of non-ionic surfactant, preferably from 2 wt % to 8 wt %, more preferably from 3 wt % to 7 wt %, even more preferably less than 5 wt % of non-ionic surfactant. Suitable non-ionic surfactants are well known in the art and may comprise alcohol ethoxylates, and/or alkyl ethoxylates, and/or alkylphenol ethoxylates, and/or glucamides such as fatty acid N-glucosyl N-methyl amides, and/or alkyl polyglucosides and/or mono- or diethanolamides or fatty acid amides. The detergent composition may also comprise from 0 wt % to 10 wt % of cationic surfactant, preferably from 0.1 wt % to 8 wt %, more preferably from 0.5 wt % to 7 wt %, even more preferably less than 5 wt % of cationic surfactant. Suitable cationic surfactants are well known in the art and may comprise alkyl quaternary ammonium compounds, and/or alkyl pyridinium compounds and/or alkyl quaternary phosphonium compounds and/or alkyl ternary sulphonium compounds. The composition preferably comprises surfactant in an amount to provide from 100 ppm to 5,000 ppm surfactant in the wash liquor during the laundering process. The composition upon contact with water typically forms a wash liquor comprising from 0.5 g/l to 10 g/l detergent composition. Many suitable surface active compounds are available and fully described in the literature, for example, in “Surface- Active Agents and Detergents”, Volumes I and 11, by Schwartz, Perry and Berch.

Builders

The main role of builder is to sequester divalent metal ions (such as calcium and magnesium ions) from the wash solution that would otherwise interact negatively with the surfactant system. Builders are also effective at removing metal ions and inorganic soils from the fabric surface, leading to improved removal of particulate and beverage stains. Builders are also a source of alkalinity and buffer the pH of the wash water to a level of 9.5 to 11. The buffering capacity is also termed reserve alkalinity, and should preferably be greater than 4.

The detergent compositions of the present invention may comprise one or more detergent builders or builder systems. Many suitable builder systems are described in the literature, for example in Powdered Detergents, Surfactant science series volume 71, Marcel Dekker, Inc. Builder may comprise from 0% to 60%, preferably from 5% to 45%, more preferably from 10% to 40%, most preferably from 15% to 35%, even more preferably from 20% to 30% builder by weight of the subject composition. The composition may comprise from 0% to 15%, preferably from 1% to 12%, 2% to 10%, most preferably from 3% to 8%, even most preferably from 4% to 6% of builder by weight of the subject composition.

Builders include, but are not limited to, the alkali metal, ammonium and alkanolammonium salts of polyphosphates (e.g., tripolyphosphate STPP), alkali metal silicates, alkaline earth and alkali metal carbonates, aluminosilicate builders (e.g., zeolite) and polycarboxylate compounds, ether hydroxypolycarboxylates, copolymers of maleic anhydride with ethylene or vinyl methyl ether, 1,3,5-trihydroxy benzene-2,4,6-trisulphonic acid, and carboxymethyloxysuccinic acid, the various alkali metal, ammonium and substituted ammonium salts of polyacetic acids such as ethylenediamine tetraacetic acid and nitrilotriacetic acid, as well as polycarboxylates such as mellitic acid, succinic acid, citric acid, oxydisuccinic acid, polymaleic acid, benzene 1,3,5-tricarboxylic acid, carboxymethyloxysuccinic acid, and soluble salts thereof. Ethanole amines (MEA, DEA, and TEA) may also contribute to the buffering capacity in liquid detergents.

Bleaches

The detergent compositions of the present invention may comprise one or more bleaching agents. In particular powdered detergents may comprise one or more bleaching agents. Suitable bleaching agents include other photobleaches, pre-formed peracids, sources of hydrogen peroxide, bleach activators, hydrogen peroxide, bleach catalysts and mixtures thereof. In general, when a bleaching agent is used, the compositions of the present invention may comprise from about 0.1% to about 50% or even from about 0.1% to about 25% bleaching agent by weight of the subject cleaning composition. Examples of suitable bleaching agents include:

(1) other photobleaches for example Vitamin K3;

(2) preformed peracids: Suitable preformed peracids include, but are not limited to, compounds selected from the group consisting of percarboxylic acids and salts, percarbonic acids and salts, perimidic acids and salts, peroxymonosulfuric acids and salts, for example, Oxone, and mixtures thereof. Suitable percarboxylic acids include hydrophobic and hydrophilic peracids having the formula R—(C═O)O—O—M wherein R is an alkyl group, optionally branched, having, when the peracid is hydrophobic, from 6 to 14 carbon atoms, or from 8 to 12 carbon atoms and, when the peracid is hydrophilic, less than 6 carbon atoms or even less than 4 carbon atoms; and M is a counterion, for example, sodium, potassium or hydrogen;

(3) sources of hydrogen peroxide, for example, inorganic perhydrate salts, including alkali metal salts such as sodium salts of perborate (usually mono- or tetra-hydrate), percarbonate, persulphate, perphosphate, persilicate salts and mixtures thereof. In one aspect of the invention the inorganic perhydrate salts are selected from the group consisting of sodium salts of perborate, percarbonate and mixtures thereof. When employed, inorganic perhydrate salts are typically present in amounts of from 0.05 to 40 wt %, or 1 to 30 wt % of the overall composition and are typically incorporated into such compositions as a crystalline solid that may be coated. Suitable coatings include inorganic salts such as alkali metal silicate, carbonate or borate salts or mixtures thereof, or organic materials such as water-soluble or dispersible polymers, waxes, oils or fatty soaps. Useful bleaching compositions are described in U.S. Pat. Nos. 5,576,282, and 6,306,812;

(4) bleach activators having R—(C═O)-L wherein R is an alkyl group, optionally branched, having, when the bleach activator is hydrophobic, from 6 to 14 carbon atoms, or from 8 to 12 carbon atoms and, when the bleach activator is hydrophilic, less than 6 carbon atoms or even less than 4 carbon atoms; and L is leaving group. Examples of suitable leaving groups are benzoic acid and derivatives thereof—especially benzene sulphonate. Suitable bleach activators include dodecanoyl oxybenzene sulphonate, decanoyl oxybenzene sulphonate, decanoyl oxybenzoic acid or salts thereof, 3,5,5-trimethyl hexanoyloxybenzene sulphonate, tetraacetyl ethylene diamine (TAED) and nonanoyloxybenzene sulphonate (NOBS). Suitable bleach activators are also disclosed in WO 98/17767. While any suitable bleach activator may be employed, in one aspect of the invention the subject cleaning composition may comprise NOBS, TAED or mixtures thereof; and

(5) bleach catalysts that are capable of accepting an oxygen atom from peroxyacid and transferring the oxygen atom to an oxidizable substrate are described in WO 2008/007319. Suitable bleach catalysts include, but are not limited to: iminium cations and polyions; iminium zwitterions; modified amines; modified amine oxides; N-sulphonyl imines; N-phosphonyl imines; N-acyl imines; thiadiazole dioxides; perfluoroimines; cyclic sugar ketones and mixtures thereof. The bleach catalyst will typically be comprised in the detergent composition at a level of from 0.0005% to 0.2%, from 0.001% to 0.1%, or even from 0.005% to 0.05% by weight.

When present, the peracid and/or bleach activator is generally present in the composition in an amount of from about 0.1 to about 60 wt %, from about 0.5 to about 40 wt % or even from about 0.6 to about 10 wt % based on the composition. One or more hydrophobic peracids or precursors thereof may be used in combination with one or more hydrophilic peracid or precursor thereof.

The amounts of hydrogen peroxide source and peracid or bleach activator may be selected such that the molar ratio of available oxygen (from the peroxide source) to peracid is from 1:1 to 35:1, or even 2:1 to 10:1.

Adjunct materials

Dispersants—The detergent compositions of the present invention can also contain dispersants. In particular powdered detergents may comprise dispersants. Suitable water-soluble organic materials include the homo- or co-polymeric acids or their salts, in which the polycarboxylic acid comprises at least two carboxyl radicals separated from each other by not more than two carbon atoms.

Dye Transfer Inhibiting Agents—The detergent compositions of the present invention may also include one or more dye transfer inhibiting agents. Suitable polymeric dye transfer inhibiting agents include, but are not limited to, polyvinylpyrrolidone polymers, polyamine N-oxide polymers, copolymers of N-vinylpyrrolidone and N-vinylimidazole, polyvinyloxazolidones and polyvinylimidazoles or mixtures thereof. When present in a subject composition, the dye transfer inhibiting agents may be present at levels from about 0.0001% to about 10%, from about 0.01% to about 5% or even from about 0.1% to about 3% by weight of the composition.

Fluorescent whitening agent—The detergent compositions of the present invention will preferably also contain additional components that may tint articles being cleaned, such as fluorescent whitening agent or optical brighteners. Any fluorescent whitening agent suitable for use in a laundry detergent composition may be used in the composition of the present invention. The most commonly used fluorescent whitening agents are those belonging to the classes of diaminostilbene-sulphonic acid derivatives, diarylpyrazoline derivatives and bisphenyl-distyryl derivatives.

Preferred fluorescent whitening agents are Tinopal DMS and Tinopal CBS available from Ciba-Geigy AG, Basel, Switzerland. Tinopal DMS is the disodium salt of 4, 4′-bis-(2-morpholino-4 anilino-s-triazin-6-ylamino) stilbene disulphonate. Tinopal CBS is the disodium salt of 2,2′-bis-(phenyl-styryl) disulphonate.

Also preferred are fluorescent whitening agents is the commercially available Parawhite KX, supplied by Paramount Minerals and Chemicals, Mumbai, India.

Other fluorescers suitable for use in the invention include the 1-3-diaryl pyrazolines and the 7-alkylaminocoumarins.

Suitable fluorescent brightener levels include lower levels of from about 0.01, from 0.05, from about 0.1 or even from about 0.2 wt % to upper levels of 0.5 or even 0.75 wt %.

Fabric hueing agents—The detergent compositions of the present invention may also include fabric hueing agents such as dyes or pigments which when formulated in detergent compositions can deposit onto a fabric when said fabric is contacted with a wash liquor comprising said detergent compositions thus altering the tint of said fabric through absorption of visible light. Fluorescent whitening agents emit at least some visible light. In contrast, fabric hueing agents alter the tint of a surface as they absorb at least a portion of the visible light spectrum. Suitable fabric hueing agents include dyes and dye-clay conjugates, and may also include pigments. Suitable dyes include small molecule dyes and polymeric dyes. Suitable small molecule dyes include small molecule dyes selected from the group consisting of dyes falling into the Colour Index (C.I.) classifications of Direct Blue, Direct Red, Direct Violet, Acid Blue, Acid Red, Acid Violet, Basic Blue, Basic Violet and Basic Red, or mixtures thereof, for example as described in WO 2005/03274, WO 2005/03275, WO 2005/03276 and EP 1 876 226. The detergent composition preferably comprises from about 0.00003 wt % to about 0.2 wt %, from about 0.00008 wt % to about 0.05 wt %, or even from about 0.0001 wt % to about 0.04 wt % fabric hueing agent. The composition may comprise from 0.0001 wt % to 0.2 wt % fabric hueing agent, this may be especially preferred when the composition is in the form of a unit dose pouch.

Soil release polymers—The detergent compositions of the present invention may also include one or more soil release polymers which aid the removal of soils from fabrics such as cotton and polyester based fabrics, in particular the removal of hydrophobic soils from polyester based fabrics. The soil release polymers may for example be nonionic or anionic terephthalte based polymers, polyvinyl caprolactam and related copolymers, vinyl graft copolymers, polyester polyamides see for example Chapter 7 in Powdered Detergents, Surfactant science series, volume 71, Marcel Dekker, Inc. Another type of soil release polymers are amphiphilic alkoxylated grease cleaning polymers comprising a core structure and a plurality of alkoxylate groups attached to that core structure. The core structure may comprise a polyalkylenimine structure or a polyalkanolamine structure as described in detail in WO 2009/087523. Furthermore random graft co-polymers are suitable soil release polymers Suitable graft co-polymers are described in more detail in WO 2007/138054, WO 2006/108856 and WO 2006/113314. Other soil release polymers are substituted polysaccharide structures especially substituted cellulosic structures such as modified cellulose deriviatives such as those described in EP 1 867 808 or WO 2003/040279. Suitable cellulosic polymers include cellulose, cellulose ethers, cellulose esters, cellulose amides and mixtures thereof. Suitable cellulosic polymers include anionically modified cellulose, nonionically modified cellulose, cationically modified cellulose, zwitterionically modified cellulose, and mixtures thereof. Suitable cellulosic polymers include methyl cellulose, carboxy methyl cellulose, ethyl cellulose, hydroxyl ethyl cellulose, hydroxyl propyl methyl cellulose, ester carboxy methyl cellulose, and mixtures thereof.

Anti-redeposition agents—The detergent compositions of the present invention may also include one or more anti-redeposition agents such as carboxymethylcellulose (CMC), polyvinyl alcohol (PVA), polyvinylpyrrolidone (PVP), polyoxyethylene and/or polyethyleneglycol (PEG), homopolymers of acrylic acid, copolymers of acrylic acid and maleic acid, and ethoxylated polyethyleneimines. The cellulose based polymers described under soil release polymers above may also function as anti-redeposition agents.

Other suitable adjunct materials include, but are not limited to, anti-shrink agents, anti-wrinkling agents, bactericides, binders, carriers, dyes, enzyme stabilizers, fabric softeners, fillers, foam regulators, hydrotropes, perfumes, pigments, sod suppressors, solvents, structurants for liquid detergents and/or structure elasticizing agents.

In one aspect the detergent is a compact fluid laundry detergent composition comprising: a) at least about 10%, preferably from 20 to 80% by weight of the composition, of surfactant selected from anionic surfactants, non ionic surfactants, soap and mixtures thereof; b) from about 1% to about 30%, preferably from 5 to 30%, by weight of the composition, of water; c) from about 1% to about 15%, preferably from 3 to 10% by weight of the composition, of non-aminofunctional solvent; and d) from about 5% to about 20%, by weight of the composition, of a performance additive selected from chelants, soil release polymers, enzymes and mixtures thereof; wherein the compact fluid laundry detergent composition comprises at least one of:

(i) the surfactant has a weight ratio of the anionic surfactant to the nonionic surfactant from about 1.5:1 to about 5:1, the surfactant comprises from about 15% to about 40%, by weight of the composition, of anionic surfactant and comprises from about 5% to about 40%, by weight of the composition, of the soap; (ii) from about 0.1% to about 10%, by weight of the composition, of a suds boosting agent selected from suds boosting polymers, cationic surfactants, zwitterionic surfactants, amine oxide surfactants, amphoteric surfactants, and mixtures thereof; and (ii) both (i) and (ii). All the ingredients are described in WO 2007/130562. Further polymers useful in detergent formulations are described in WO 2007/149806.

In another aspect the detergent is a compact granular (powdered) detergent comprising a) at least about 10%, preferably from 15 to 60% by weight of the composition, of surfactant selected from anionic surfactants, non-ionic surfactants, soap and mixtures thereof; b) from about 10 to 80% by weight of the composition, of a builder, preferably from 20% to 60% where the builder may be a mixture of builders selected from i) phosphate builder, preferably less than 20%, more preferably less than 10% even more preferably less than 5% of the total builder is a phosphate builder; ii) a zeolite builder, preferably less than 20%, more preferably less than 10% even more preferably less than 5% of the total builder is a zeolite builder; iii) citrate, preferably 0 to 5% of the total builder is a citrate builder; iv) polycarboxylate, preferably 0 to 5% of the total builder is a polycarboxylate builder v) carbonate, preferably 0 to 30% of the total builder is a carbonate builder and vi) sodium silicates, preferably 0 to 20% of the total builder is a sodium silicate builder; c) from about 0% to 25% by weight of the composition, of fillers such as sulphate salts, preferably from 1% to 15%, more preferably from 2% to 10%, more preferably from 3% to 5% by weight of the composition, of fillers; and d) from about 0.1% to 20% by weight of the composition, of enzymes, preferably from 1% to 15%, more preferably from 2% to 10% by weight of the composition, of enzymes.

The soils and stains that are important for detergent formulators are composed of many different substances, and a range of different enzymes, all with different substrate specificities have been developed for use in detergents both in relation to laundry and hard surface cleaning, such as dishwashing. These enzymes are considered to provide an enzyme detergency benefit, since they specifically improve stain removal in the cleaning process they are applied in as compared to the same process without enzymes. Stain removing enzymes that are known in the art include enzymes such as carbohydrases, amylases, proteases, lipases, cellulases, hemicellulases, xylanases, cutinases, and pectinase.

In a preferred aspect of the present invention the beta-glucanase of the invention (e.g. a variant of the present invention) may be combined with at least two enzymes. These additional enzymes are described in details in the section “other enzymes”, more preferred at least three, four or five enzymes. Preferably, the enzymes have different substrate specificity, e.g., carbolytic activity, proteolytic activity, amylolytic activity, lipolytic activity, hemicellulytic activity or pectolytic activity. The enzyme combination may for example be a beta-glucanase of the invention (e.g. a variant of the present invention) with another stain removing enzyme, e.g., a beta-glucanase of the invention and a protease, a beta-glucanase of the invention and a serine protease, a beta-glucanase of the invention and an amylase, a beta-glucanase of the invention and a cellulase, beta-glucanase of the invention and a lipase, a beta-glucanase of the invention and a cutinase, a beta-glucanase of the invention and a pectinase or a beta-glucanase of the invention and an anti-redeposition enzyme. More preferably, the beta-glucanase of the invention is combined with at least two other stain removing enzymes, e.g., a beta-glucanase of the invention, a lipase and an amylase; or a beta-glucanase of the invention, a protease and an amylase; or a beta-glucanase of the invention, a protease and a lipase; or a beta-glucanase of the invention, a protease and a pectinase; or a beta-glucanase of the invention, a protease and a cellulase; or a beta-glucanase of the invention, a protease and a hemicellulase; or a beta-glucanase of the invention, a protease and a cutinase; or a beta-glucanase of the invention, an amylase and a pectinase; or a beta-glucanase of the invention, an amylase and a cutinase; or a beta-glucanase of the invention, an amylase and a cellulase; or a beta-glucanase of the invention, an amylase and a hemicellulase; or a beta-glucanase of the invention, a lipase and a pectinase; or a beta-glucanase of the invention, a lipase and a cutinase; or a beta-glucanase of the invention, a lipase and a cellulase; or a beta-glucanase of the invention, a lipase and a hemicellulase. Even more preferably, a beta-glucanase of the invention may be combined with at least three other stain removing enzymes, e.g., a beta-glucanase of the invention, a protease, a lipase and an amylase; or a beta-glucanase of the invention, a protease, an amylase and a pectinase; or a beta-glucanase of the invention, a protease, an amylase and a cutinase; or a beta-glucanase of the invention, a protease, an amylase and a cellulase; or a beta-glucanase of the invention, a protease, an amylase and a hemicellulase; or a beta-glucanase of the invention, an amylase, a lipase and a pectinase; or a beta-glucanase of the invention, an amylase, a lipase and a cutinase; or a beta-glucanase of the invention, an amylase, a lipase and a cellulase; or a beta-glucanase of the invention, an amylase, a lipase and a hemicellulase; or a beta-glucanase of the invention, a protease, a lipase and a pectinase; or a beta-glucanase of the invention, a protease, a lipase and a cutinase; or a beta-glucanase of the invention, a protease, a lipase and a cellulase; or a beta-glucanase of the invention, a protease, a lipase and a hemicellulase. A beta-glucanase according to the present invention may be combined with any of the enzymes selected from the non-exhaustive list comprising: carbohydrases, such as an amylase, a hemicellulase, a pectinase, a cellulase, a xanthanase or a pullulanase, a peptidase, a protease or a lipase.

In a preferred embodiment, a beta-glucanase of the invention (e.g. a variant of the present invention) is combined with a serine protease, e.g., an S8 family protease such as Savinase®.

In another embodiment of the present invention, a beta-glucanase of the invention may be combined with one or more metalloproteases, such as an M4 metalloprotease, including Neutrase® or Thermolysin. Such combinations may further comprise combinations of the other detergent enzymes as outlined above.

The cleaning process or the textile care process may for example be a laundry process, a dishwashing process or cleaning of hard surfaces such as bathroom tiles, floors, table tops, drains, sinks and washbasins. Laundry processes can for example be household laundering, but it may also be industrial laundering. Furthermore, the invention relates to a process for laundering of fabrics and/or garments where the process comprises treating fabrics with a washing solution containing a detergent composition, and at least one beta-glucanase of the invention (e.g. a variant of the present invention). The cleaning process or a textile care process can for example be carried out in a machine washing process or in a manual washing process. The washing solution can for example be an aqueous washing solution containing a detergent composition.

The fabrics and/or garments subjected to a washing, cleaning or textile care process of the present invention may be conventional washable laundry, for example household laundry. Preferably, the major part of the laundry is garments and fabrics, including knits, woven, denims, non-woven, felts, yarns, and towelling. The fabrics may be cellulose based such as natural cellulosics, including cotton, flax, linen, jute, ramie, sisal or coir or manmade cellulosics (e.g., originating from wood pulp) including viscose/rayon, ramie, cellulose acetate fibers (tricell), lyocell or blends thereof. The fabrics may also be non-cellulose based such as natural polyamides including wool, camel, cashmere, mohair, rabit and silk or synthetic polymer such as nylon, aramid, polyester, acrylic, polypropylen and spandex/elastane, or blends thereof as well as blend of cellulose based and non-cellulose based fibers. Examples of blends are blends of cotton and/or rayon/viscose with one or more companion material such as wool, synthetic fibers (e.g., polyamide fibers, acrylic fibers, polyester fibers, polyvinyl alcohol fibers, polyvinyl chloride fibers, polyurethane fibers, polyurea fibers, aramid fibers), and cellulose-containing fibers (e.g., rayon/viscose, ramie, flax, linen, jute, cellulose acetate fibers, lyocell).

The last few years there has been an increasing interest in replacing components in detergents, which is derived from petrochemicals with renewable biological components such as enzymes and polypeptides without compromising the wash performance. When the components of detergent compositions change new enzyme activities or new enzymes having alternative and/or improved properties compared to the common used detergent enzymes such as proteases, lipases and amylases is needed to achieve a similar or improved wash performance when compared to the traditional detergent compositions.

Typical detergent compositions includes various components in addition to the enzymes, these components have different effects, some components like the surfactants lower the surface tension in the detergent, which allows the stain being cleaned to be lifted and dispersed and then washed away, other components like bleach systems removes discolor often by oxidation and many bleaches also have strong bactericidal properties, and are used for disinfecting and sterilizing. Yet other components like builder and chelator softens, e.g., the wash water by removing the metal ions from the liquid.

In a particular embodiment, the invention concerns the use of a composition comprising a beta-glucanase of the invention (e.g. a variant of the present invention), wherein said enzyme composition further comprises at least one or more of the following a surfactant, a builder, a chelator or chelating agent, bleach system or bleach component in laundry or dish wash.

In a preferred embodiment of the invention the amount of a surfactant, a builder, a chelator or chelating agent, bleach system and/or bleach component are reduced compared to amount of surfactant, builder, chelator or chelating agent, bleach system and/or bleach component used without the added beta-glucanase of the invention. Preferably the at least one component which is a surfactant, a builder, a chelator or chelating agent, bleach system and/or bleach component is present in an amount that is 1% less, such as 2% less, such as 3% less, such as 4% less, such as 5% less, such as 6% less, such as 7% less, such as 8% less, such as 9% less, such as 10% less, such as 15% less, such as 20% less, such as 25% less, such as 30% less, such as 35% less, such as 40% less, such as 45% less, such as 50% less than the amount of the component in the system without the addition of beta-glucanase of the invention (e.g. a variant of the present invention), such as a conventional amount of such component. In one aspect, the beta-glucanase of the invention (e.g. a variant of the present invention) is used in detergent compositions wherein said composition is free of at least one component which is a surfactant, a builder, a chelator or chelating agent, bleach system or bleach component and/or polymer.

Washing Method

The detergent compositions of the present invention are ideally suited for use in laundry applications. Accordingly, the present invention includes a method for laundering a fabric. The method comprises the steps of contacting a fabric to be laundered with a cleaning laundry solution comprising the detergent composition according to the invention. The fabric may comprise any fabric capable of being laundered in normal consumer use conditions. The solution preferably has a pH of from about 5.5 to about 8, further preferably pH selected in the range from about 7.5 to about 13.5, or in the range from about 7.5 to about 12.5, or in the range from about 8.5 to about 11.5, or in the range from about 9.5 to about 10.5, or pH 7.5 or above.

A preferred embodiment concerns a method of cleaning, the method comprising the steps of: contacting an object with a high pH cleaning composition (e.g. pH 7.5 or above) comprising a beta-glucanase of the invention (e.g. a variant of the present invention) under conditions suitable for cleaning the object. In a preferred embodiment the cleaning composition is used in a laundry or a dish wash process.

Still another embodiment relates to a method for removing stains from fabric or dishware which comprises contacting the fabric or dishware with a high pH cleaning composition (e.g. pH 7.5 or above) comprising a beta-glucanase of the invention (e.g. a variant of the present invention) under conditions suitable for cleaning the object.

Also contemplated are compositions and methods of treating fabrics (e.g., to desize a textile) using the cleaning composition of the invention. The high pH cleaning composition can be used in any fabric-treating method which is well known in the art.

In another embodiment the high pH cleaning composition of the present invention is suited for use in liquid laundry and liquid hard surface applications, including dish wash and car wash. Accordingly, the present invention includes a method for laundering a fabric or washing a hard surface. The method comprises the steps of contacting the fabric/dishware to be cleaned with a solution comprising the high pH cleaning composition according to the invention. The fabric may comprise any fabric capable of being laundered in normal consumer use conditions. The hard surface may comprise any dishware such as crockery, cutlery, ceramics, plastics such as melamine, metals, china, glass, acrylics or other hard surfaces such as cars, floors etc. The solution preferably has a pH, e.g. 7.5 or above, e.g. from about 9 to about 13.5.

The compositions may be employed at concentrations of from about 100 ppm, preferably 500 ppm to about 15,000 ppm in solution. The water temperatures typically range from about 5° C. to about 90° C., including about 10° C., about 15° C., about 20° C., about 25° C., about 30° C., about 35° C., about 40° C., about 45° C., about 50° C., about 55° C., about 60° C., about 65° C., about 70° C., about 75° C., about 80° C., about 85° C. and about 90° C. The water to fabric ratio is typically from about 1:1 to about 30:1.

In particular embodiments, the washing method is conducted at a pH of from about 5.0 to about 11.5, or in alternative embodiments, even from about 6 to about 10.5, such as about 5 to about 11, about 5 to about 10, about 5 to about 9, about 5 to about 8, about 5 to about 7, about 5.5 to about 11, about 5.5 to about 10, about 5.5 to about 9, about 5.5 to about 8, about 5.5. to about 7, about 6 to about 11, about 6 to about 10, about 6 to about 9, about 6 to about 8, about 6 to about 7, about 6.5 to about 11, about 6.5 to about 10, about 6.5 to about 9, about 6.5 to about 8, about 6.5 to about 7, about 7 to about 11, about 7 to about 10, about 7 to about 9, or about 7 to about 8, preferably about 5.5 to about 9, and more preferably about 6 to about 8. In preferred embodiments the washing method is conducted at a pH selected in the range from about 7.5 to about 13.5, or in the range from about 7.5 to about 12.5, or in the range from about 8.5 to about 11.5, or in the range from about 9.5 to about 10.5, or pH 7.5 or above.

In some preferred embodiments, the high pH cleaning compositions provided herein are typically formulated such that, during use in aqueous cleaning operations, the wash water has a pH of from about 9 to about 13.5, or in alternative embodiments, or from about 10 to about 13.5 even from about 11 to about 13.5. In some preferred embodiments the liquid laundry products are formulated to have a pH from about 12 to about 13.5. Techniques for controlling pH at recommended usage levels include the use of buffers, acids, alkalis, etc., and are well known to those skilled in the art. In the context of the present invention alkalis are used to adjust pH to about 9 to 13.5 preferably about 10 to 13.5.

In particular embodiments, the washing method is conducted at a degree of hardness of from about 0° dH to about 30° dH, such as about 1° dH, about 2° dH, about 3° dH, about 4° dH, about 5° dH, about 6° dH, about 7° dH, about 8° dH, about 9° dH, about 10° dH, about 11° dH, about 12° dH, about 13° dH, about 14° dH, about 15° dH, about 16° dH, about 17° dH, about 18° dH, about 19° dH, about 20° dH, about 21° dH, about 22° dH, about 23° dH, about 24° dH, about 25° dH, about 26° dH, about 27° dH, about 28° dH, about 29° dH, about 30° dH. Under typical European wash conditions, the degree of hardness is about 15° dH, under typical US wash conditions about 6° dH, and under typical Asian wash conditions, about 3° dH.

The present invention relates to a method of cleaning a fabric, a dishware or hard surface with a detergent composition comprising a beta-glucanase of the invention (e.g. a variant of the present invention).

A preferred embodiment concerns a method of cleaning, said method comprising the steps of: contacting an object with a cleaning composition comprising a beta-glucanase of the invention (e.g. a variant of the present invention) under conditions suitable for cleaning said object. In a preferred embodiment the cleaning composition is a detergent composition and the process is a laundry or a dish wash process.

Still another embodiment relates to a method for removing stains from fabric which comprises contacting said a fabric with a composition comprising a beta-glucanase of the invention (e.g. a variant of the present invention) under conditions suitable for cleaning said object.

Low Temperature Uses

One embodiment of the invention concerns a method of doing laundry, dish wash or industrial cleaning comprising contacting a surface to be cleaned with a beta-glucanase of the invention (e.g. a variant of the present invention), and wherein said laundry, dish wash, industrial or institutional cleaning is performed at a temperature of about 40° C. or below. One embodiment of the invention relates to the use of a beta-glucanase (e.g. a variant of the present invention) in laundry, dish wash or a cleaning process wherein the temperature in laundry, dish wash, industrial cleaning is about 40° C. or below

In another embodiment, the invention concerns the use of a beta-glucanase according to the invention (e.g. a variant of the present invention) in a beta-glucan removing process, wherein the temperature in the beta-glucan removing process is about 40° C. or below.

In each of the above-identified methods and uses, the wash temperature is about 40° C. or below, such as about 39° C. or below, such as about 38° C. or below, such as about 37° C. or below, such as about 36° C. or below, such as about 35° C. or below, such as about 34° C. or below, such as about 33° C. or below, such as about 32° C. or below, such as about 31° C. or below, such as about 30° C. or below, such as about 29° C. or below, such as about 28° C. or below, such as about 27° C. or below, such as about 26° C. or below, such as about 25° C. or below, such as about 24° C. or below, such as about 23° C. or below, such as about 22° C. or below, such as about 21° C. or below, such as about 20° C. or below, such as about 19° C. or below, such as about 18° C. or below, such as about 17° C. or below, such as about 16° C. or below, such as about 15° C. or below, such as about 14° C. or below, such as about 13° C. or below, such as about 12° C. or below, such as about 11° C. or below, such as about 10° C. or below, such as about 9° C. or below, such as about 8° C. or below, such as about 7° C. or below, such as about 6° C. or below, such as about 5° C. or below, such as about 4° C. or below, such as about 3° C. or below, such as about 2° C. or below, such as about 1° C. or below.

In another preferred embodiment, the wash temperature is in the range of about 5-40° C., such as about 5-30° C., about 5-20° C., about 5-10° C., about 10-40° C., about 10-30° C., about 10-20° C., about 15-40° C., about 15-30° C., about 15-20° C., about 20-40° C., about 20-30° C., about 25-40° C., about 25-30° C., or about 30-40° C. In particular preferred embodiments the wash temperature is about 20° C., about 30° C., or about 40° C.

High Temperature Uses

One embodiment of the invention concerns a method of doing laundry, dish wash or industrial cleaning comprising contacting a surface to be cleaned with a beta-glucanase of the invention (e.g. a variant of the present invention), and wherein said laundry, dish wash, industrial or institutional cleaning is performed at a temperature of about 75° C. or below. One embodiment of the invention relates to the use of a beta-glucanase in laundry, dish wash or a cleaning process wherein the temperature in laundry, dish wash, industrial cleaning is about 70° C. or below.

In another embodiment, the invention concerns the use of a beta-glucanase according to the invention (e.g. a variant of the present invention) in a beta-glucan removing process, wherein the temperature in the beta-glucan removing process is about 65° C. or below.

In each of the above-identified methods and uses, the wash temperature is about 60° C. or below, such as about 59° C. or below, such as about 58° C. or below, such as about 57° C. or below, such as about 56° C. or below, such as about 55° C. or below, such as about 54° C. or below, such as about 53° C. or below, such as about 52° C. or below, such as about 51° C. or below, such as about 50° C. or below, such as about 49° C. or below, such as about 48° C. or below, such as about 47° C. or below, such as about 46° C. or below, such as about 45° C. or below, such as about 44° C. or below, such as about 43° C. or below, such as about 42° C. or below, such as about 41° C. or below.

In another preferred embodiment, the wash temperature is in the range of about 41-90° C., such as about 41-80° C., about 41-85° C., about 41-80° C., about 41-75° C., about 41-70° C., about 41-65° C., about 41-60° C.

The Invention is Further Defined in the Following Paragraphs:
1. A variant of a parent beta-glucanase, the variant comprising a substitution at one or more positions corresponding to positions 33 and 188 of the mature polypeptide of SEQ ID NO: 26 using the numbering of SEQ ID NO: 26, wherein the variant has beta-glucanase activity and wherein the variant has at least 60%, e.g., at least 61%, at least 62%, at least 63%, at least 64%, at least 65%, at least 66%, at least 67%, at least 68%, at least 69%, at least 70%, at least 71%, at least 72%, at least 73%, at least 74%, at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 95.5%, at least 96%, at least 96.5%, at least 97%, at least 97.5%, at least 98%, at least 98.5%, at least 99%, or at least 99.5%, but less than 100% sequence identity to the mature polypeptide of any of: SEQ ID NO: 26, SEQ ID NO: 27, SEQ ID NO: 25, and SEQ ID NO: 28, preferably the variant comprising a substitution at one or more positions corresponding to positions F33 and M188 of the mature polypeptide of SEQ ID NO: 26 using the numbering of SEQ ID NO: 26, further preferably said beta-glucanase activity is not an endo-cellulase activity on β-1,4 linkages between D-glucose units of cellulose.
2. The variant of paragraph 1, which is a variant of a parent beta-glucanase selected from the group consisting of:

a. a polypeptide having at least 60%, e.g., at least 61%, at least 62%, at least 63%, at least 64%, at least 65%, at least 66%, at least 67%, at least 68%, at least 69%, at least 70%, at least 71%, at least 72%, at least 73%, at least 74%, at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 95.5%, at least 96%, at least 96.5%, at least 97%, at least 97.5%, at least 98%, at least 98.5%, at least 99%, or at least 99.5%, or 100% sequence identity to the mature polypeptide selected from the group consisting of: SEQ ID NO: 26, SEQ ID NO: 27, SEQ ID NO: 25 and SEQ ID NO: 28;

b. a polypeptide encoded by a polynucleotide that hybridizes under low stringency conditions, preferably medium stringency conditions, further preferably medium-high stringency conditions, further most preferably high stringency conditions, further most preferably very high stringency conditions, with (i) the mature polypeptide coding sequence of the sequence selected from the group consisting of: SEQ ID NO: 6, SEQ ID NO: 1, SEQ ID NO: 4, SEQ ID NO: 8, or (ii) the full-length complement of (i) or (ii);

c. a polypeptide encoded by a polynucleotide having at least 60%, e.g., at least 61%, at least 62%, at least 63%, at least 64%, at least 65%, at least 66%, at least 67%, at least 68%, at least 69%, at least 70%, at least 71%, at least 72%, at least 73%, at least 74%, at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 95.5%, at least 96%, at least 96.5%, at least 97%, at least 97.5%, at least 98%, at least 98.5%, at least 99%, or at least 99.5%, sequence identity to the mature polypeptide coding sequence of the sequence selected from the group consisting of: SEQ ID NO: 6, SEQ ID NO: 1, SEQ ID NO: 4, SEQ ID NO: 8, and

d. a fragment of the mature polypeptide selected from the group consisting of: SEQ ID NO: 26, SEQ ID NO: 27, SEQ ID NO: 25 and SEQ ID NO: 28, wherein said fragment has beta-glucanase activity.

3. The variant of any of paragraphs 1-2, wherein the parent beta-glucanase comprises or consists of the polypeptide selected from the group consisting of: SEQ ID NO: 26, SEQ ID NO: 27, SEQ ID NO: 25 and SEQ ID NO: 28.

4. The variant of any of paragraphs 1-3, wherein the number of alterations is 1-20, e.g., 1-10 and 1-5, such as 1, 2, 3, 4, 5, 6, 7, 8, 9 or 10 alterations.

5. The variant of any of paragraphs 1-4, which comprises a substitution at a position corresponding to position 33, e.g., F33, wherein the substituent amino acid is any of: Ala, Arg, Asn, Asp, Cys, Gln, Glu, Gly, His, Ile, Leu, Lys, Phe, Pro, Ser, Thr, Trp, Tyr or Val, preferably the substituent amino acid is selected from the group consisting of: Ala, Asn, Cys, Gln, Glu, Gly, Leu, Ser, Trp, Tyr or Val, further preferably the substituent amino acid is selected from the group consisting of: Val, Gly, Asn, Ser or Cys.

6. The variant of any of paragraphs 1-5, which comprises a substitution at a position corresponding to position 188, e.g., M188, wherein the substituent amino acid is any of: Ala, Arg, Asn, Asp, Cys, Gln, Glu, Gly, His, Ile, Leu, Lys, Phe, Pro, Ser, Thr, Trp, Tyr or Val, preferably the substituent amino acid is selected from the group consisting of: Ala, Arg, Cys, Gln, Glu, His, Leu, Phe, Pro, Ser, Thr or Tyr, further preferably the substituent amino acid is selected from the group consisting of: Leu, His or Arg;

7. The variant of any of paragraphs 1-6, which comprises or consists of a substitution selected from the group consisting of: F33V+M188L; F33A+M188F; F33Y; F33V+M188H; F33G+M188L; F33N; F33G+M188R; F33S+M188Y; F33G+M188H; F33E+M188L; M188H; F33W+M188S; F33N+M188F; F33S+M188A; F33C+M188L; F33V+M188T; F33Q+M188R; F33L+M188T; F33G+M188C; F33N+M188Q; F33L+M188A.

8. The variant of any of paragraphs 1-7, which has an improved property relative to the parent, wherein the improved property is increased oxidation stability.

9. The variant of any of paragraphs 1-8, wherein the variant consists of 180 to 230, e.g., 190 to 222, 195 to 205 amino acids.

10. The variant of any of paragraphs 1-9, wherein said variant is capable of having beta-glucanase activity in an aqueous solution with a pH selected in the range from about 7.5 to about 13.5, wherein said aqueous solution optionally comprises a bleaching agent, preferably said pH is selected in the range from about 7.5 to about 12.5, further preferably said pH is selected in the range from about 8.5 to about 11.5, most preferably said pH is selected in the range from about 9.5 to about 10.5.

11. The variant of any of paragraphs 1-10, wherein said variant is capable of having beta-glucanase activity in an aqueous solution at a temperature selected in the range from about 20° C. to about 75° C., wherein said aqueous solution optionally comprises a bleaching agent, preferably said temperature is selected in the range from about 40° C. to about 60° C.

12. The variant of any of paragraphs 1-11, wherein said variant is capable of having beta-glucanase activity for at least 15 minutes, preferably for at least 30 minutes, further preferably for at least 60 minutes, further most preferably for at least 90 minutes, further most preferably for at least 120 minutes.

13. The variant of any of paragraphs 1-12, wherein said beta-glucanase activity comprises licheninase EC 3.2.1.73 activity.

14. A composition comprising a variant of any of the paragraphs 1-13.

15. The composition of paragraph 14, further comprising one or more detergent components.

16. The composition of paragraph 15, wherein the detergent component is selected from the group consisting of: surfactants, hydrotropes, builders, co-builders, chelators, bleach components, polymers, fabric hueing agents, fabric conditioners, foam boosters, suds suppressors, dispersants, dye transfer inhibitors, fluorescent whitening agents, perfume, optical brighteners, bactericides, fungicides, soil suspending agents, soil release polymers, anti-redeposition agents, enzyme inhibitors, enzyme stabilizers, enzyme activators, antioxidants, and solubilizers.

17. The composition of any of paragraphs 14-16, further comprising one or more additional enzymes, preferably said one or more additional enzymes is one or more amylases, further preferably said one or more amylases is one or more alpha-amylases.

18. The composition of any of paragraphs 14-17, further comprising an enzyme selected from the group consisting of: DNases, perhydrolases, amylases, proteases, peroxidases, cellulases, betaglucanases, xyloglucanases, hemicellulases, xanthanases, xanthan lyases, lipases, acyl transferases, phospholipases, esterases, laccases, catalases, aryl esterases, amylases, alpha-amylases, glucoamylases, cutinases, pectinases, pectate lyases, keratinases, reductases, oxidases, phenoloxidases, lipoxygenases, ligninases, carrageenases, pullulanases, tannases, arabinosidases, hyaluronidases, chondroitinases, xyloglucanases, xylanases, pectin acetyl esterases, polygalacturonases, rhamnogalacturonases, other endo-beta-mannanases, exo-beta-mannanases, pectin methylesterases, cellobiohydrolases, transglutaminases, and combinations thereof.

19. The composition of any of paragraphs 14-18, wherein said composition has pH of 7.5 or above and optionally, comprises a bleaching agent; preferably said pH is selected in the range from about 7.5 to about 13.5, further preferably said pH is selected in the range from about 7.5 to about 12.5, most preferably said pH is selected in the range from about 8.5 to about 11.5, further most preferably said pH is selected in the range from about 9.5 to about 10.5.

20. The composition of any of paragraphs 14-19, wherein said composition has improved stability and/or performance under alkaline conditions, preferably said alkaline conditions have pH 7.5 or above.

21. The composition of any of paragraphs 14-20, wherein said composition is a cleaning or detergent composition.

22. Use of a variant of any of paragraphs 1-13 or a composition of any of paragraphs 14-21 for degrading a beta-glucan, preferably said beta-glucan is a beta-D-glucan, further preferably said beta-glucan is a beta-1,3-1,4 glucan, most preferably said beta-glucan is a mix-linkage beta-glucan, further most preferably said beta-glucan is a barley beta-glucan or oatmeal beta-glucan; optionally said use is carried out under alkaline conditions having pH 7.5 or above.

23. Use of a variant of any of paragraphs 1-13 or a composition of any of paragraphs 14-21 for washing or cleaning a textile and/or a hard surface such as dish wash including Automatic Dish Wash (ADW); optionally said use is carried out under alkaline conditions having pH 7.5 or above.

24. Use of a variant of any of paragraphs 1-13 or a composition of any of paragraphs 14-21 in a cleaning process such as laundry or hard surface cleaning including dish wash including Automatic Dish Wash (ADW) and industrial cleaning; optionally said use is carried out under alkaline conditions having pH 7.5 or above.

25. Use of a variant of any of paragraphs 1-13 or a composition of any of paragraphs 14-21 for laundering and/or hard surface cleaning including dish wash including Automatic Dish Wash (ADW), wherein said polypeptide or said composition has an enzyme detergency benefit; optionally said use is carried out under alkaline conditions having pH 7.5 or above.

26. Use of a variant of any of paragraphs 1-13 or a composition of any of paragraphs 14-21 for at least one of the following: preventing, reducing or removing a biofilm from an item, preferably a malodor is reduced or removed from said item; optionally said use is carried out under alkaline conditions having pH 7.5 or above.

27. A process of degrading a beta-glucan comprising applying a variant of any of paragraphs 1-13 or a composition of any of paragraphs 14-21 to said beta-glucan, preferably said beta-glucan is a beta-D-glucan, further preferably said beta-glucan is a beta-1,3-1,4 glucan, most preferably said beta-glucan is a mix-linkage beta-glucan, further most preferably said beta-glucan is a barley beta-glucan or oatmeal beta-glucan; optionally, said process is carried out under alkaline conditions having pH 7.5 or above.

28. The process of paragraph 27, wherein said beta-glucan is on the surface of a textile or hard surface, such as dish wash.

29. A fermentation broth formulation or cell culture composition comprising a variant of any of paragraphs 1-13.

30. A polynucleotide encoding a variant of any of paragraphs 1-13.

31. A nucleic acid construct or expression vector capable of expressing a polynucleotide of paragraph 30, preferably said nucleic acid construct or said expression vector comprising the polynucleotide of paragraph 30 operably linked to one or more control sequences that direct the production of the polypeptide in an expression host.

32. A recombinant host cell comprising the polynucleotide of paragraph 30, preferably said polynucleotide is operably linked to one or more control sequences that direct the production of the polypeptide, further preferably said recombinant host cell is an isolated recombinant host cell.

33. A composition comprising at least one of the following: i) a polynucleotide of paragraph 30; or ii) a nucleic acid construct of paragraph 31; or iii) an expression vector of paragraph 31.

34. A method of producing a beta-glucanase variant, comprising cultivating the recombinant host cell of paragraph 32 under conditions suitable for expression of the variant.

35. The method of paragraph 34 further comprising recovering the variant.

36. A transgenic plant, plant part or plant cell transformed with a polynucleotide encoding a variant of any of paragraphs 1-13.

37. A method for producing a beta-glucanase variant, comprising cultivating the transgenic plant or plant cell of paragraph 36 under conditions conducive for production of the polypeptide.

38. The method of paragraph 37, further comprising recovering the variant.

39. A method for obtaining a beta-glucanase variant, comprising: introducing into a parent beta-glucanase a substitution at one or more positions corresponding to positions 33, e.g., F33, and 188, e.g., M188, of the mature polypeptide of SEQ ID NO: 26 using the numbering of SEQ ID NO: 26, wherein the variant has beta-glucanase activity; and recovering the variant.

40. The method of paragraph 39, wherein the parent has at least 60%, e.g., at least 61%, at least 62%, at least 63%, at least 64%, at least 65%, at least 66%, at least 67%, at least 68%, at least 69%, at least 70%, at least 71%, at least 72%, at least 73%, at least 74%, at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 95.5%, at least 96%, at least 96.5%, at least 97%, at least 97.5%, at least 98%, at least 98.5%, at least 99%, or at least 99.5%, or 100% sequence identity to a mature polypeptide selected from the group consisting of: SEQ ID NO: 26, SEQ ID NO: 27, SEQ ID NO: 25 and SEQ ID NO: 28.

41. The method of any of paragraphs 34-35 and 39-40, wherein the parent beta-glucanase is obtained or is obtainable from a Bacillus sp.

42. A cleaning or detergent composition comprising a variant of any of the paragraphs 1-13 and one or more amylases, preferably said variant and said one or more amylases have a synergistic effect; further preferably said synergistic effect is a REM synergistic effect, further most preferably said REM synergistic effect is of more than 6.5 at about 40° C. for about 30 minutes at pH of about 7.5, further most preferably said REM synergistic effect is of more than 6.1 at about 40° C. for about 30 minutes at pH of about 10, further most preferably said REM synergistic effect is of more than 6.2 at about 40° C. for about 30 minutes at pH of about 10, further most preferably said beta-glucanase activity is not an endo-cellulase activity on β-1,4 linkages between D-glucose units of cellulose.

43. The cleaning or detergent composition of paragraph 42, wherein said variant is capable of having beta-glucanase activity in an aqueous solution with a pH in the range from about 7.5 to about 13.5, wherein said aqueous solution optionally comprises a bleaching agent, preferably said pH is in the range from about 7.5 to about 12.5, further preferably said pH is in the range from about 8.5 to about 11.5, most preferably said pH is in the range from about 9.5 to about 10.5.

44. The cleaning or detergent composition of any of paragraphs 42-43, wherein said variant is capable of showing beta-glucanase activity in an aqueous solution at a temperature selected in the range from about 20° C. to about 75° C., and/or in the range from about 40° C. to about 60° C., wherein said aqueous solution optionally comprises a bleaching agent.

45. The cleaning or detergent composition of any of paragraphs 42-44, wherein said variant is capable of having beta-glucanase activity for at least 15 minutes, preferably for at least 30 minutes, further preferably for at least 60 minutes, further most preferably for at least 90 minutes, further most preferably for at least 120 minutes.

46. The cleaning or detergent composition of any of paragraphs 42-45, wherein said beta-glucanase activity comprises alkaline beta-glucanase activity, wherein said alkaline beta-glucanase activity is beta-glucanase activity at pH 7.5 or above.

47. The cleaning or detergent composition of any of paragraphs 42-46, wherein said beta-glucanase activity comprises licheninase EC 3.2.1.73 activity, preferably said beta-glucanase activity is licheninase EC 3.2.1.73 activity.

48. The cleaning or detergent composition of any of paragraphs 42-47, wherein said amylase is an alpha-amylase.

49. The cleaning or detergent composition of any of paragraphs 42-48, further comprising one or more detergent components.

50. The cleaning or detergent composition of paragraph 49, wherein the detergent component is selected from the group consisting of: surfactants, hydrotropes, builders, co-builders, chelators, bleach components, polymers, fabric hueing agents, fabric conditioners, foam boosters, suds suppressors, dispersants, dye transfer inhibitors, fluorescent whitening agents, perfume, optical brighteners, bactericides, fungicides, soil suspending agents, soil release polymers, anti-redeposition agents, enzyme inhibitors, enzyme stabilizers, enzyme activators, antioxidants, and solubilizers.

51. The cleaning or detergent composition of any of paragraphs 42-50, further comprising one or more additional enzymes.

52. The cleaning or detergent composition of any of paragraphs 42-51, further comprising an enzyme selected from the group consisting of: DNases, perhydrolases, amylases, proteases, peroxidases, cellulases, betaglucanases, xyloglucanases, hemicellulases, xanthanases, xanthan lyases, lipases, acyl transferases, phospholipases, esterases, laccases, catalases, aryl esterases, amylases, alpha-amylases, glucoamylases, cutinases, pectinases, pectate lyases, keratinases, reductases, oxidases, phenoloxidases, lipoxygenases, ligninases, carrageenases, pullulanases, tannases, arabinosidases, hyaluronidases, chondroitinases, xyloglucanases, xylanases, pectin acetyl esterases, polygalacturonases, rhamnogalacturonases, other endo-beta-mannanases, exo-beta-mannanases, pectin methylesterases, cellobiohydrolases, transglutaminases, and combinations thereof.

53. The cleaning or detergent composition of any of paragraphs 42-52, wherein said composition has pH of 7.5 or above and optionally, comprises a bleaching agent; preferably said pH is selected in the range from about 7.5 to about 13.5, further preferably said pH is selected in the range from about 7.5 to about 12.5, most preferably said pH is selected in the range from about 8.5 to about 11.5, further most preferably said pH is selected in the range from about 9.5 to about 10.5.

54. The cleaning or detergent composition of any of paragraphs 42-53, wherein said alpha-amylase is selected from the group consisting of:

(a) a polypeptide having at least 90% sequence identity to SEQ ID NO: 13 (corresponding to SEQ ID NO: 2 of WO 95/10603);

(b) a polypeptide having at least 90% sequence identity to SEQ ID NO: 13 (corresponding to SEQ ID NO: 2 in WO 95/10603), wherein the polypeptide comprises a substitution in one or more of positions: 15, 23, 105, 106, 124, 128, 133, 154, 156, 178, 179, 181, 188, 190, 197, 201, 202, 207, 208, 209, 211, 243, 264, 304, 305, 391, 408, and/or 444;

(c) a polypeptide having at least 90% sequence identity to SEQ ID NO: 14 (corresponding to SEQ ID NO: 6 in WO 02/010355);

(d) a polypeptide having at least 90% sequence identity to the hybrid polypeptide of SEQ ID NO: 15 (comprising residues 1-33 of SEQ ID NO: 6 of WO 2006/066594 and residues 36-483 of SEQ ID NO: 4 of WO 2006/066594);

(e) a polypeptide having at least 90% sequence identity to the hybrid polypeptide of SEQ ID NO: 15 (comprising residues 1-33 of SEQ ID NO: 6 of WO 2006/066594 and residues 36-483 of SEQ ID NO: 4 of WO 2006/066594), wherein the hybrid polypeptide comprises a substitution, a deletion or an insertion in one of more of positions: 48, 49, 107, 156, 181, 190, 197, 201, 209 and/or 264;

(f) a polypeptide having at least 90% sequence identity to SEQ ID NO: 16 (corresponding to SEQ ID NO: 6 of WO 02/019467);

(g) a polypeptide having at least 90% sequence identity to SEQ ID NO: 16 (corresponding to SEQ ID NO: 6 of WO 02/019467), wherein the polypeptide comprises a substitution, a deletion or an insertion in one of more of positions: 181, 182, 183, 184, 195, 206, 212, 216 and/or 269;

(h) a polypeptide having at least 90% sequence identity to SEQ ID NO: 17, SEQ ID NO: 18 or SEQ ID NO: 19 (corresponding to SEQ ID NO: 1, SEQ ID NO: 2 or SEQ ID NO: 7 of WO 96/023873)

(i) a polypeptide having at least 90% sequence identity to SEQ ID NO: 17, SEQ ID NO: 18 or SEQ ID NO: 19 (corresponding to SEQ ID NO: 1, SEQ ID NO: 2 or SEQ ID NO: 7 of WO 96/023873), wherein the polypeptide comprises a substitution, a deletion or an insertion in one of more of positions: 140, 183, 184 195, 206, 243, 260, 304 and/or 476;

(j) a polypeptide having at least 90% sequence identity to SEQ ID NO: 20 (corresponding to SEQ ID NO: 2 of WO 08/153815);

(k) a polypeptide having at least 90% sequence identity to SEQ ID NO: 21 (corresponding to SEQ ID NO: 10 of WO 01/66712);

(l) a polypeptide having at least 90% sequence identity to SEQ ID NO: 21 (corresponding to SEQ ID NO: 10 of WO 01/66712), wherein the polypeptide comprises a substitution, a deletion or an insertion in one of more of positions: 176, 177, 178, 179, 190, 201, 207, 211 and/or 264;

(m) a polypeptide having at least 90% sequence identity to SEQ ID NO: 22 (corresponding to SEQ ID NO: 2 of WO 09/061380);

(n) a polypeptide having at least 90% sequence identity to SEQ ID NO: 22 (corresponding to SEQ ID NO: 2 of WO 09/061380), wherein the polypeptide comprises a substitution, a deletion or an insertion in one of more of positions: 87, 98, 125, 128, 131, 165, 178, 180, 181, 182, 183, 201, 202, 225, 243, 272, 282, 305, 309, 319, 320, 359, 444 and/or 475;

(o) a polypeptide having at least 90% sequence identity to SEQ ID NO: 21, wherein the polypeptide comprises a substitution, a deletion or an insertion in one of more of positions: 28, 118, 174; 181, 182, 183, 184, 186, 189, 195, 202, 298, 299, 302, 303, 306, 310, 314; 320, 324, 345, 396, 400, 439, 444, 445, 446, 449, 458, 471 and/or 484;

(p) a polypeptide having at least 90% sequence identity to SEQ ID NO: 12;

(r) a polypeptide having at least 90% sequence identity to SEQ ID NO: 12 (corresponding to SEQ ID NO: 2 in WO 95/10603), wherein the polypeptide comprises a substitution in one or more of positions: 15, 23, 105, 106, 124, 128, 133, 154, 156, 178, 179, 181, 188, 190, 197, 201, 202, 207, 208, 209, 211, 243, 264, 304, 305, 391, 408, and/or 444;

(s) a polypeptide having at least 90% sequence identity to SEQ ID NO: 29; and

(t) a polypeptide having at least 90% sequence identity to SEQ ID NO: 29, wherein the polypeptide comprises a substitution in one or more of positions: 187, 203, 476, 458, 459, 460, 178, 179, 180, 181, 7, 200, 126, 132, 303, 477, 15, 23, 105, 106, 124, 128, 133, 154, 156, 178, 179, 181, 188, 190, 197, 201, 202, 207, 208, 209, 211, 243, 264, 304, 305, 391, 408, and/or 444.

55. The cleaning or detergent composition of any of paragraphs 42-54, wherein said composition has improved stability and/or performance under alkaline conditions, preferably said alkaline conditions have pH 7.5 or above.

56. The cleaning or detergent composition of any of paragraphs 42-55, wherein said composition is in form selected from a group consisting of: a bar, a homogenous tablet, a tablet having two or more layers, a pouch having one or more compartments, a regular or compact powder, a granule, a paste, a gel, or a regular, compact or concentrated liquid.

57. The cleaning or detergent composition of any of paragraphs 42-56, having an enzyme detergency benefit in cleaning or detergent applications.

58. The cleaning or detergent composition of any of paragraphs 42-57 having improved stability and/or performance, preferably said improved stability and/or performance is under alkaline conditions having pH 7.5 or above.

59. A method for removing a stain from a surface which comprises contacting the surface with a composition according to any of paragraphs 42-58.

60. Use of the cleaning or detergent composition of any of paragraphs 42-58 for degrading a beta-glucan, preferably said beta-glucan is a beta-D-glucan, further preferably said beta-glucan is a beta-1,3-1,4 glucan, most preferably said beta-glucan is a mix-linkage beta-glucan, further most preferably said beta-glucan is a barley beta-glucan or oatmeal beta-glucan; optionally said use is carried out under alkaline conditions having pH 7.5 or above.

61. Use the cleaning or detergent composition of any of paragraphs 42-58 for washing or cleaning a textile and/or a hard surface such as dish wash including Automatic Dish Wash (ADW); optionally said use is carried out under alkaline conditions having pH 7.5 or above.

62. Use the cleaning or detergent composition of any of paragraphs 42-58 in a cleaning process such as laundry or hard surface cleaning including dish wash including Automatic Dish Wash (ADW) and industrial cleaning; optionally said use is carried out under alkaline conditions having pH 7.5 or above.

63. Use the cleaning or detergent composition of any of paragraphs 42-58 for laundering and/or hard surface cleaning including dish wash including Automatic Dish Wash (ADW), wherein said composition has an enzyme detergency benefit; optionally said use is carried out under alkaline conditions having pH 7.5 or above.

64. Use the cleaning or detergent composition of any of paragraphs 42-58 for at least one of the following: preventing, reducing or removing a biofilm from an item, preferably a malodor is reduced or removed from said item; optionally said use is carried out under alkaline conditions having pH 7.5 or above.

65. A process of degrading a beta-glucan comprising applying the cleaning or detergent composition of any of paragraphs 42-58 to said beta-glucan, preferably said beta-glucan is a beta-D-glucan, further preferably said beta-glucan is a beta-1,3-1,4 glucan, most preferably said beta-glucan is a mix-linkage beta-glucan, further most preferably said beta-glucan is a barley beta-glucan or oatmeal beta-glucan; optionally, said process is carried out under alkaline conditions having pH 7.5 or above.

66. The process of paragraph 65, wherein said beta-glucan is on the surface of a textile or hard surface, such as dish wash.

The present invention is further described by the following examples that should not be construed as limiting the scope of the invention.

### EXAMPLES

### Detergent Compositions Used in the Example Sections as Described Herein Included the Following:

TABLE A
Model detergent A

| | Content of | Active component |
|---|---|---|
| Compound | compound (% w/w) | (% w/w) |
| LAS | 12.0 | 97 |
| AEOS, SLES | 17.6 | 28 |
| Soy fatty acid | 2.8 | 90 |
| Coco fatty acid | 2.8 | 99 |
| AEO | 11.0 | 100 |
| Sodium hydroxide | 1.8 | 99 |
| Ethanol/Propan-2-ol | 3.0 | 90/10 |
| MPG | 6.0 | 98 |
| Glycerol | 1.7 | 99.5 |
| TEA | 3.3 | 100 |
| Sodium formate | 1.0 | 95 |
| Sodium citrate | 2.0 | 100 |
| DTMPA (as Na7-salt) | 0.5 | 42 |
| PCA (as Na-salt) | 0.5 | 40 |
| Phenoxy ethanol | 0.5 | 99 |
| Ion exchanged water | 33.6 | — |

Water hardness was adjusted to 15°dH by addition of CaCl₂, MgCl₂, and NaHCO₃ (Ca²⁺:Mg²⁺:HCO₃₋ = 4:1:7.5) to the test system.

TABLE B
Model detergent X

| | Content of | Active component |
|---|---|---|
| Compound | compound (% w/w) | (% w/w) |
| LAS | 16.5 | 91 |
| AEO* | 2 | 99.5 |
| Sodium carbonate | 20 | 100 |
| Sodium (di)silicate | 12 | 82.5 |
| Zeolite A | 15 | 80 |
| Sodium sulfate | 33.5 | 100 |
| PCA | 1 | 100 |

*Model detergent X was mixed without AEO. AEO was added separately before wash. Water hardness was adjusted to 12°dH by addition of CaCl₂, MgCl₂, and NaHCO₃ (Ca²⁺:Mg²⁺:HCO₃₋ = 2:1:4.5) to the test system.

TABLE C
Model detergent Z without bleach

| | Content of | % active component |
|---|---|---|
| Compound | compound (% w/w) | (% w/w) |
| LAS | 7.0 | 85.3 |
| Soap | 1.1 | 93 |
| AEO* | 1.5 | 99.5 |
| Soda ash | 20.1 | 99.5 |
| Hydrous sodium silicate | 10.0 | 80.1 |
| Zeolite A | 5.0 | 80 |
| Sodium citrate | 2.0 | 100 |
| HEDP-Na4 | 0.2 | 84 |
| Polyacrylate | 1.1 | 92 |
| Sodium sulfate | 52.0 | 100 |

*Model detergent Z without bleach was mixed without AEO. AEO was added separately before wash. Water hardness was adjusted to 15° dH by addition of CaCl₂, MgCl₂, and NaHCO₃ (Ca²⁺:Mg²⁺:HCO₃₋ = 4:1:7.5) to the test system. pH was used as is (10.6) or adjusted to 11.3 with 4M NaOH.

TABLE D
Model detergent Z with bleach

| | Content of | % active component |
|---|---|---|
| Compound | compound (% w/w) | (% w/w) |
| LAS | 7.0 | 85.3 |
| Soap | 1.1 | 93 |
| AEO* | 1.5 | 99.5 |
| Soda ash | 20.1 | 99.5 |
| Hydrous sodium silicate | 10.0 | 80.1 |
| Zeolite A | 5.0 | 80 |
| Sodium citrate | 2.0 | 100 |
| HEDP-Na4 | 0.2 | 84 |
| Polyacrylate | 1.1 | 92 |
| Sodium percarbonate | 9.3 | 86 |
| TEAD | 1.1 | 91.8 |
| Sodium sulfate | 41.6 | 100 |

*Model detergent Z with bleach was mixed without AEO. AEO was added separately before wash. Water hardness was adjusted to 15° dH by addition of CaCl₂, MgCl₂, and NaHCO₃ (Ca²⁺:Mg²⁺:HCO₃₋ = 4:1:7.5) to the test system. pH was either as is (10.5) or adjusted to 11.1 with 4M NaOH.

TABLE E
Automatic Dish Wash (ADW) model detergent A

| | Content of | Active component |
|---|---|---|
| Compound | compound (% w/w) | (% w/w) |
| MGDA (Trilon M Granules SG) | 20 | 59 |
| Sodium citrate | 20 | 100 |
| Sodium carbonate | 20 | 100 |
| Sodium percarbonate | 10 | 88 |
| Sodium Silicate | 5 | 80 |
| Sodium sulfate | 12 | 100 |
| Acusol 588G | 5 | 92 |
| TAED | 3 | 92 |
| Surfac 23-6.5 (liq) | 5 | 100 |

Water hardness was adjusted to 21° dH by addition of CaCl₂, MgCl₂, and NaHCO₃ (Ca²⁺:Mg²⁺:HCO₃₋ = 4:1:10) to the test system.

### Example 1

### Determination of Beta-Glucanase Activity

An AZCL-Barley beta-glucan (azurine dye covalently cross-linked beta-glucan) assay was used for detection of endo-glucancase activity. AZCL-Barley beta-glucan (75 mg) was suspended in 15 mL detergent (Model detergents A, X, Z with and without bleach and pH adjusted, ADW Model A). To 1 mL of this solution in Eppendorf tubes was added 10 μL enzyme (0.33 mg enzyme protein/Liter), incubated for 15 min at 40° C. while shaking at 1250 rpm in a pre-heated thermo mixer and spun down for 2 min at 13200 rpm, diluted 5 times with a 5% Triton-X-100 including 10 μM CaCl₂ and 250 μL of the solution was transferred to a micro-titer plate and the sample absorbance was measured at 590 nm.

### Example 2

### Cloning, Expression and Purification of GH16 Endo-β-1,3-1,4-Glucanase from the Genus Bacillus

The beta-glucanases were derived from bacterial strains obtain either from the German collection of Microorganisms and Cell Cultures (DSMZ) or by isolation from environmental samples by classical microbiological techniques according to Table 1.

TABLE 1
Source and Source country of GH16 endo- β-1,3-
1,4-glucanase from the genus Bacillus

| Strain name | Source | Source Country |
|---|---|---|
| Bacillus sp-62449 | Environmental sample | United States |
| Bacillus akibai | Soil | Greece |
| Bacillus agaradhaerens | Soil | United States |
| Bacillus mojavensis | DSMZ (DSM9205) | United States |

Chromosomal DNA from pure cultures of the individual strains was purified and subjected to full genome sequencing using Illumina technology. The assembled genome sequence and subsequent analysis of the 16S ribosomal subunit gene sequences confirmed the identity of the strains.

The individual genes encoding β-1,3-1,4-glucanases were amplified by PCR and fused with regulatory elements and homology regions for recombination into the B. subtilis genome.

The linear integration construct was a SOE-PCR fusion product (Horton, R. M., Hunt, H. D., Ho, S. N., Pullen, J. K. and Pease, L. R. (1989) Engineering hybrid genes without the use of restriction enzymes, gene splicing by overlap extension Gene 77: 61-68) made by fusion of the gene between two Bacillus subtilis chromosomal regions along with strong promoters and a chloramphenicol resistance marker. The SOE PCR method is also described in patent application WO 2003095658.

The gene was expressed under the control of a triple promoter system (as described in WO 99/43835), consisting of the promoters from Bacillus licheniformis alpha-amylase gene (amyL), Bacillus amyloliquefaciens alpha-amylase gene (amyQ), and the Bacillus thuringiensis cryIIIA promoter including stabilizing sequence.

The gene was expressed with a Bacillus clausii secretion signal (encoding the following amino acid sequence: MKKPLGKIVASTALLISVAFSSSIASA (SEQ ID NO: 10) replacing the native secretion signal. Furthermore the expression construct results in the addition of an N-terminal poly histidine affinity purification tag consisting of the sequence HHHHHHPR (SEQ ID NO: 11) to the expressed mature protein.

The SOE-PCR product was transformed into Bacillus subtilis and integrated in the chromosome by homologous recombination into the pectate lyase locus. Subsequently, a recombinant Bacillus subtilis clone containing the integrated expression construct was grown in rich liquid culture. The culture broth was centrifuged (20000×g, 20 min) and the supernatant was carefully decanted from the precipitate and used for purification of the enzyme.

### Purification of Recombinant Enzymes by Nickel Affinity Chromatography

The pH of the cleared supernatant was adjusted to pH 8, filtrated through a 0.2 μM filter, and the supernatant applied to a 5 ml HisTrap™ excel column. Prior to loading, the column had been equilibrated in 5 column volumes (CV) of 50 mM Tris/HCl pH 8. In order to remove unbound material, the column was washed with 8 CV of 50 mM Tris/HCl pH 8, and elution of the target was obtained with 50 mM HEPES pH 7+10 mM imidazole. The eluted protein was desalted on a HiPrep™ 26/10 desalting column, equilibrated using 3 CV of 50 mM HEPES pH 7+100 mM NaCl. This buffer was also used for elution of the target, and the flow rate was 10 ml/min. Relevant fractions were selected and pooled based on the chromatogram and SDS-PAGE analysis.

### Example 3

### AZCL-Assay with Beta-Glucanase Enzymes

In this example enzymatic activity were measured on AZCL-Barely beta-glucan substrate under various pH's, temperature and detergent thus modeling various laundry conditions. Measurements of enzymatic activity were carried out as described in example 1, but without the 5 times dilution with 5% Triton-X-100 including 10 μM CaCl₂. Comparisons were made with beta-glucanase from Bacillus amyloliquefaciens and beta-glucanase from Bacillus subtilis in Model detergent A, Model detergent X, Model detergent Z with bleach, Model detergent Z without bleach, Model detergent Z with bleach pH-adjusted and Model Z without bleach pH-adjusted detergent compositions.

TABLE 2
Beta-glucanase activity measured under various pH's, temperatures and
laundry detergents using the AZCL-Barley beta-glucan assay (Absorbance):

| | | | | | | | | | pH 11.1 | | | |
|---|---|---|---|---|---|---|---|---|---|---|---|---|
| | | | | | | | | | Model Z | | pH 11.3 | |
| | | | | | pH 10.5 | | pH 10.6 | | with | | Model Z | |
| | | | | | Model Z | | Model Z | | bleach | | without | |
| | pH 7.7 | | pH 10.1 | | with | | without | | pH- | | bleach pH- | |
| | Model A | | Model X | | bleach | | bleach | | adjusted | | adjusted | |
| | 40° | 60° | 40° | 60° | 40° | 60° | 40° | 60° | 40° | 60° | 40° | 60° |
| Enzyme | C. | C. | C. | C. | C. | C. | C. | C. | C. | C. | C. | C. |
| B. | 2.44 | 0.71 | 2.83 | 0.83 | 0.05 | 0.04 | 0.10 | 0.01 | 0.01 | 0.03 | 0.07 | 0.01 |
| amyloliquefaciens | | | | | | | | | | | | |
| beta- | | | | | | | | | | | | |
| glucanase | | | | | | | | | | | | |
| (lichenase) | | | | | | | | | | | | |
| B. subtilis | 2.45 | 0.62 | 3.41 | 0.30 | 0.05 | 0.01 | 0.08 | 0.01 | 0.00 | 0.04 | 0.07 | 0.02 |
| beta- | | | | | | | | | | | | |
| glucanase | | | | | | | | | | | | |
| (lichenase) | | | | | | | | | | | | |
| B. akibai | 0.18 | 0.10 | 3.41 | 1.55 | 0.03 | 0.37 | 0.05 | 0.27 | 0.03 | 0.15 | 0.04 | 0.05 |
| Beta- | | | | | | | | | | | | |
| glucanase | | | | | | | | | | | | |
| (lichenase) | | | | | | | | | | | | |
| B. agaradhaerens | 0.36 | 0.70 | 3.41 | 2.50 | 0.58 | 0.16 | 0.47 | 0.04 | 0.17 | 0.03 | 0.01 | 0.02 |
| beta- | | | | | | | | | | | | |
| glucanase | | | | | | | | | | | | |
| (lichenase) | | | | | | | | | | | | |
| B. sp-62449 | 1.22 | 1.15 | 3.25 | 0.08 | 0.22 | 0.10 | 0.30 | 0.11 | 0.05 | 0.04 | 0.04 | 0.01 |
| beta- | | | | | | | | | | | | |
| glucanase | | | | | | | | | | | | |
| (lichenase) | | | | | | | | | | | | |
| B. mojavensis | 1.65 | 0.20 | 3.41 | 2.36 | 0.17 | 0.11 | 0.18 | 0.01 | 0.03 | 0.03 | 0.01 | 0.02 |
| beta- | | | | | | | | | | | | |
| glucanase | | | | | | | | | | | | |
| (lichenase) | | | | | | | | | | | | |

For details of the model detergent compositions see Tables A-E above.

### Example 4

### AZCL-Assay of Enzyme Activity on AZCL-Beta-Barley Substrate in Automated Dish Wash Model Detergent

Measurements of enzymatic activity were carried out as described in example 1. In this example enzymatic activities of novel beta-glucanases were compared to enzymatic activities of beta-glucanases from Bacillus amyloliquefaciens and Bacillus subtilis in the automated dish wash detergent model A. The obtained data are shown in Table 3 below:

TABLE 3
Beta-glucanase activity measured under various temperatures
in ADW Model A detergent using the AZCL-Barley
beta-glucan assay (Absorbance), pH 10.2:

| | ADW model detergent A | |
|---|---|---|
| Enzyme | 40° C. | 60° C. |
| Blank | 0.07 | 0.11 |
| Bacillus amyloliquefaciens beta- | 0.46 | 0.34 |
| glucanase (lichenase) | | |
| Bacillus subtilis beta-glucanase | 0.42 | 0.21 |
| (lichenase) | | |
| Bacillus akibai beta-glucanase | 0.15 | 2.07 |
| (lichenase) | | |
| Bacillus agaradhaerens beta-glucanase | 0.85 | 1.77 |
| (lichenase) | | |
| Bacillus mojavensis beta-glucanase | 0.85 | 1.06 |
| (lichenase) | | |
| Bacillus sp-62449 beta-glucanase | 1.60 | 0.49 |
| (lichenase) | | |

### Example 5

### Beta-Glucanase Stability Measured by TSA

In this example stability of novel beta-glucanases were compared to stabilities of beta-glucanases from Bacillus amyloliquefaciens and Bacillus subtilis. Thermal shift assays (TSA) were performed with enzyme samples diluted to 0.3 mg/ml in assay buffers: 0.1 M succinic acid, 0.1 M HEPES, 0.1 M CHES, 0.1 M CAPS, 0.15 M KCl, 1 mM CaCl2, 0.01% Triton X100, pH adjusted to 5, 7.5 and 10 respectively. SYPRO Orange dye (Life Technologies S6650) diluted 101× in mQ water. 10 μl diluted enzyme sample+10 μl assay buffer+10 μl dye were mixed in wells of TSA assay plates (LightCycler 480 Multiwell plate 96, white (Roche) and covered with optic seal (LightCycler 480 Sealing foil, Roche). Protein melting analysis was conducted at 25-99° C. at 200° C./h in a Roche Lightcycler 480 II machine running Roche LightCycler 480 software (release 1.5.0 SP4). All samples were analyzed in duplicate. The reported readout is Tm, defined as the midpoint value of the protein melting curves. The obtained data are shown in Table 4 below.

TABLE 4
Stability measured by TSA

| Enzyme | Buffer pH | TSA |
|---|---|---|
| Bacillus akibai beta-glucanase | 5 | 70.9 |
| (lichenase) | 7.5 | 71.8 |
| | 10 | 71.6 |
| Bacillus agaradhaerens beta-glucanase | 5 | 58.2 |
| (lichenase) | 7.5 | 64.0 |
| | 10 | 58.6 |
| Bacillus mojavensis beta-glucanase | 5 | 72.8 |
| (lichenase) | 7.5 | 71.2 |
| | 10 | 72.2 |
| Bacillus sp-62449 beta-glucanase | 5 | 43.2 |
| (lichenase) | 7.5 | 53.9 |
| | 10 | 49.4 |
| Bacillus amyloliquefaciens beta-glucanase | 5 | 72.8 |
| (lichenase) | 7.5 | 70.1 |
| | 10 | 73.2 |
| Bacillus subtilis beta-glucanase | 5 | 64.2 |
| (lichenase) | 5 | 64.7 |
| | 7.5 | 64.8 |

### Example 6

### Beta-Glucanase Substrate Specificity

The substrate specificities of beta-glucanases were further tested using various AZCL-assays from Megazymes (AZCL-Barely beta-glucan, AZCL-HE-cellulose, AZCL-pachyman and AZCL-curdlan (azurine dye covalently cross-linked beta-glucan). The AZCL-substrate (75 mg) was suspended in 15 mL model detergent X. To 1 mL of this solution in Eppendorf tubes was added 10 μL enzyme (0.33 mg enzyme protein/Liter), incubated for 15 min at 40° C. while shaking at 1250 rpm in a pre-heated thermo mixer and spun down for 2 min at 13200 rpm, diluted 5 times with a 5% Triton-X-100 including 10 μM CaCl₂ and 250 μL of the solution was transferred to a micro-titer plate and the sample absorbance was measured at 590 nm.

In this example substrate specificity of all 6 beta-glucanases (i.e. from Bacillus akibai, Bacillus agaradhaerens, Bacillus mojavensis, Bacillus sp-62449, Bacillus amyloliquefaciens and Bacillus subtilis) were tested on AZCL-Barley beta-glucan, AZCL-HE-Cellulose AZCL-pachyman and AZCL-curdlan substrates. The obtained results have further confirmed that all 6 tested beta-glucanases have activity on AZCL-Barley beta-glucan substrate only (i.e. positive reaction on AZCL-Barley beta-glucan as a substrate and negative reactions on AZCL-HE-Cellulose AZCL-pachyman and AZCL-curdlan as substrates, Table 5 below). The data shows that tested beta-glucanases only showed activity on beta-glucans containing both beta-1,3 and beta-1,4 linkages and not beta-glucans consisting of pure beta-1,4-glucans or beta-1,3 glucans only or a mixture of beta-1,3- and beta-1,6 linkages. Based on the above results, beta-glucanases of the present invention can be further distinguished from endo-cellulases within beta-glucanase definition as used herein, said endo-cellulases having activity on β-1,4 linkages between D-glucose units of cellulose. Based on the above it is concluded that beta-glucanases of the present invention have licheninase (EC 3.2.1.73) enzymatic activity.

TABLE 5
Substrate specificity of 6 beta-glucanases
measured by AZCL-substrates

| | | Substrate for | |
|---|---|---|---|
| Substrate | Reaction | the assay of: | Polymer description |
| AZCL-Barley | Yes | Lichenase, endo- | β-1,4; β-1,3 linkages |
| beta-glucan | | glucanase and | between D-glucose |
| | | cellulase | units |
| AZCL-HE- | No | Endo-cellulase | β-1,4 linkages between |
| cellulose | | | D-glucose units |
| AZCL-curdlan | No | Endo-1,3-beta-D- | β-1,3 linkages between |
| | | glucanase | D-glucose |
| AZCL-pachyman | No | Endo-1,3-beta-D- | β-1,3 linkages between |
| | | glucanase | D-glucose units |
| | | | (branched with |
| | | | β-1,6 glucose units |
| | | | average on every 4) |

### Example 7

### Synergistic Effect of Beta-Glucanases (e.g. Lichenases) of the Invention when Combined with an Alpha-Amylase

I. Wascator Bottle Wash Method Description:

A Wascator bottle wash method was used to detect the performance of the enzymes. In a Wascator washing machine (FOM 71 Lab) bottles (60 mL, DSE PP 70×35 Aseptisk, material No. 216-2620, from VWR) with 25 mL detergent solution including enzyme(s) and four stains (035KC Chocolate porridge oat from Equest, 2 cm in diameter) were added. Two kg ballast (tea towels, cotton) was included in the washing machine. Washed in 25 L water for 30 min at 40° C. in liquid and powder model detergents for laundry (model A1 and model X1, respectively) and in ADW model detergent (ADW model detergent A1). After wash the stains were rinsed with tap water twice (3 L) and dried ON at rt (room temperature) in drying cabinet (Electrolux, Intuition, EDD2400). The remission was measured on a spectrophotometer (Macbeth Color-Eye 7000 Remissions) at 460 nm.

II. Results:

In this example the results of combining the individual lichenases with an alpha-amylase (Stainzyme) (SEQ ID NO: 12) were studied in order to investigate a potential synergistic effect between the two enzymes in various detergents with various pHs using the Wascator bottle wash method. Comparisons were made with lichenase from Bacillus amyloliquefaciens and lichenase from Bacillus subtilis in Model detergent A1, Model detergent X1 and ADW model detergent A1 using 0.01 mg enzyme protein per liter of lichenase and 0.05 mg enzyme protein per liter of Stainzyme at 40° C. The detailed conditions used in this example are described in Tables F-K and the results are shown in Tables 6-8 below.

TABLE F
Experimental condition

| | Model detergent A1 | |
|---|---|---|
| Detergent | (see Table G below) | |
| Detergent dosage | 3.33 | g/L |
| Test solution volume | 25 | mL |
| pH | As is | |
| Wash time | 30 | minutes |
| Temperature | 40° | C. |
| Water hardness | 15° | dH |
| Amylase concentration in test | 0.05 | mg/L |
| Beta-glucanase (Lichenase) | 0.01 | mg/L |
| concentration in test | | |

| | |
|---|---|
| Test material | O35 KC Chocolate porridge oats |

TABLE G
Model detergent A1

| | Content of | Active component |
|---|---|---|
| Compound | compound (% w/w) | (% w/w) |
| LAS | 12.0 | 97 |
| AEOS, SLES | 17.6 | 28 |
| Soy fatty acid | 2.8 | 90 |
| Coco fatty acid | 2.8 | 99 |
| AEO | 11.0 | 100 |
| Sodium hydroxide | 1.8 | 99 |
| Ethanol/Propan-2-ol | 3.0 | 90/10 |
| MPG | 6.0 | 98 |
| Glycerol | 1.7 | 99.5 |
| TEA | 3.3 | 100 |
| Sodium formate | 1.0 | 95 |
| Sodium citrate | 2.0 | 100 |
| DTMPA (as Na₇-salt) | 0.5 | 42 |
| PCA (as N₇-salt) | 0.5 | 40 |
| Phenoxy ethanol | 0.5 | 99 |
| Ion exchanged water | 33.6 | — |

Water hardness was adjusted to 15° dH by addition of CaCl2, MgCl2, and NaHCO3 (Ca2+:Mg2+:HCO3− = 4:1:7.5) to the test system.

TABLE H
Experimental condition

| | Model detergent X1 | |
|---|---|---|
| Detergent | (see Table I below) | |
| Detergent dosage | 1.75 | g/L |
| Test solution volume | 25 | mL |
| pH | As is | |
| Wash time | 30 | minutes |
| Temperature | 40° | C. |
| Water hardness | 12° | dH |
| Amylase concentration in test | 0.05 | mg/L |
| Beta-glucanase (Lichenase) | 0.01 | mg/L |
| concentration in test | | |

| | |
|---|---|
| Test material | O35 KC Chocolate porridge oats |

TABLE I
Model detergent X1

| | Content of | Active component |
|---|---|---|
| Compound | compound (% w/w) | (% w/w) |
| LAS | 16.5 | 91 |
| AEO* | 2 | 99.5 |
| Sodium carbonate | 20 | 100 |
| Sodium (di)silicate | 12 | 82.5 |
| Zeolite A | 15 | 80 |
| Sodium sulfate | 33.5 | 100 |
| PCA | 1 | 100 |

*Model detergent X1 is mixed without AEO. AEO is added separately before wash. Water hardness was adjusted to 12° dH by addition of CaCl2, MgCl2, and NaHCO3 (Ca2+:Mg2+:HCO3− = 2:1:4.5) to the test system.

TABLE J
Experimental condition

| | ADW model detergent A1 | |
|---|---|---|
| Detergent | (see Table K below) | |
| Detergent dosage | 3.77 | g/L |
| Test solution volume | 25 | mL |
| pH | As is | |
| Wash time | 30 | minutes |
| Temperature | 40° | C. |
| Water hardness | 15° | dH |
| Amylase concentration in test | 0.05 | mg/L |
| Beta-glucanase (Lichenase) | 0.01 | mg/L |
| concentration in test | | |

| | |
|---|---|
| Test material | O35 KC Chocolate porridge oats |

TABLE K
ADW model detergent A1

| | Content of | Active component |
|---|---|---|
| Compound | compound (% w/w) | (% w/w) |
| MGDA (Trilon M Granules SG) | 20 | 59 |
| Sodium citrate | 20 | 100 |
| Sodium carbonate | 20 | 100 |
| Sodium percarbonate | 10 | 88 |
| Sodium Silicate | 5 | 80 |
| Sodium sulfate | 12 | 100 |
| Acusol 588G | 5 | 92 |
| TAED | 3 | 92 |
| Surfac 23-6.5 (liq) | 5 | 100 |

Water hardness was adjusted to 21° dH by addition of CaCl2, MgCl2, and NaHCO3 (Ca2+:Mg2+:HCO3− = 4:1:10) to the test system.

### Abbreviations as Used Herein:

REM=Measured value
ΔREM=REM−Blank
REM combined=Measured value
ΔREM combined=REM combined−Blank
ΔREM theoretic=ΔREM (Amylase)+ΔREM (Lichenase)
REM Synergistic effect=ΔREM combined−ΔREM theoretic

TABLE 6
Wascator bottle wash in Model detergent A1 at 40° C., 30 min (pH 7.7):

| | | | Beta-glucanase (Lichenase) in combination | | | |
|---|---|---|---|---|---|---|
| | | | with the amylase (Stainzyme) | | | |
| | | | | | | REM |
| | Enzymes solo | | REM | ΔREM | ΔREM | Synergistic |
| | REM | ΔREM | combined | combined | theoretic | effect |
| B. agaradhaerens | 66.0 | 0.4 | 80.1 | 14.5 | 6.7 | 7.8 |
| beta-glucanase | | | | | | |
| (lichenase) | | | | | | |
| B. akibai | 65.3 | −0.2 | 79.1 | 13.6 | 6.1 | 7.5 |
| beta-glucanase | | | | | | |
| (lichenase) | | | | | | |
| B. mojavensis | 65.8 | 0.2 | 79.3 | 13.7 | 6.5 | 7.2 |
| beta-glucanase | | | | | | |
| (lichenase) | | | | | | |
| B. SP-62449 | 64.9 | −0.7 | 80.0 | 14.4 | 5.6 | 8.8 |
| beta-glucanase | | | | | | |
| (lichenase) | | | | | | |
| B. | 67.3 | 1.8 | 79.5 | 13.9 | 8.1 | 5.9 |
| amyloliquefaciens | | | | | | |
| beta-glucanase | | | | | | |
| (lichenase) | | | | | | |
| B. subtilis | 67.3 | 1.7 | 80.1 | 14.5 | 8.0 | 6.5 |
| beta-glucanase | | | | | | |
| (lichenase) | | | | | | |
| Stainzyme | 71.8 | 6.3 | — | — | — | — |
| Blank | 65.5 | 0.0 | — | — | — | — |

TABLE 7
Wascator bottle wash in Model detergent X1 at 40° C., 30 min (pH 10.1):

| | | | Beta-glucanase (Lichenase) in combination | | | |
|---|---|---|---|---|---|---|
| | | | with the amylase Stainzyme | | | |
| | | | | | | REM |
| | Enzymes solo | | REM | ΔREM | ΔREM | Synergistic |
| | REM | ΔREM | combined | combined | theoretic | effect |
| B. agaradhaerens | 61.4 | −0.4 | 74.5 | 12.7 | 4.4 | 8.2 |
| beta-glucanase | | | | | | |
| (lichenase) | | | | | | |
| B. akibai | 62.2 | 0.3 | 74.9 | 13.1 | 5.2 | 7.9 |
| beta-glucanase | | | | | | |
| (lichenase) | | | | | | |
| B. mojavensis | 61.8 | −0.1 | 74.3 | 12.4 | 4.8 | 7.6 |
| beta-glucanase | | | | | | |
| (lichenase) | | | | | | |
| B. SP-62449 | 61.9 | 0.1 | 73.0 | 11.1 | 5.0 | 6.1 |
| beta-glucanase | | | | | | |
| (lichenase) | | | | | | |
| B. | 59.9 | −1.9 | 72.0 | 10.2 | 2.9 | 7.3 |
| amyloliquefaciens | | | | | | |
| beta-glucanase | | | | | | |
| (lichenase) | | | | | | |
| B. subtilis | 60.8 | −1.0 | 71.8 | 10.0 | 3.8 | 6.1 |
| beta-glucanase | | | | | | |
| (lichenase) | | | | | | |
| Stainzyme | 66.7 | 4.9 | — | — | — | — |
| Blank | 61.8 | 0.0 | — | — | — | — |

TABLE 8
Wascator bottle wash in ADW Model detergent A1 at 40° C., 30 min (pH 10.2):

| | | | Beta-glucanase (Lichenase) in combination | | | |
|---|---|---|---|---|---|---|
| | | | with the amylase Stainzyme | | | |
| | | | | | | REM |
| | Enzymes solo | | REM | ΔREM | ΔREM | Synergistic |
| | REM | ΔREM | combined | combined | theoretic | effect |
| B. agaradhaerens | 60.5 | −2.1 | 75.1 | 12.5 | 6.1 | 6.4 |
| beta-glucanase | | | | | | |
| (lichenase) | | | | | | |
| B. akibai | 60.7 | −1.9 | 73.9 | 11.3 | 6.3 | 5.0 |
| beta-glucanase | | | | | | |
| (lichenase) | | | | | | |
| B. mojavensis | 63.0 | 0.3 | 73.3 | 10.7 | 8.5 | 2.1 |
| beta-glucanase | | | | | | |
| (lichenase) | | | | | | |
| B. SP-62449 | 60.8 | −1.8 | 74.5 | 11.9 | 6.4 | 5.5 |
| beta-glucanase | | | | | | |
| (lichenase) | | | | | | |
| B. | 61.6 | −1.0 | 71.3 | 8.6 | 7.2 | 1.4 |
| amyloliquefaciens | | | | | | |
| beta-glucanase | | | | | | |
| (lichenase) | | | | | | |
| B. subtilis | 58.1 | −4.5 | 72.5 | 9.9 | 3.7 | 6.2 |
| beta-glucanase | | | | | | |
| (lichenase) | | | | | | |
| Stainzyme | 70.8 | 8.2 | — | — | — | — |
| Blank | 62.6 | 0.0 | — | — | — | — |

### Example 8

### Determination of the pH Optimum

Subsequently, the pH optimum of all 6 beta-glucanases was determined on 0,4% w/v AZCL-glucan(barley) substrate in Britton Robinson buffer (100 mM phosphoric acid, 100 mM acetic acid, 100 mM boric acid, 0,01% Trinton X-100, 100 mM KCl, 2 mM CaCl2) adjusted to pH 2-12 with NaOH. An enzyme dilution expected to be in the high end of the linear assay range was selected for all pH values under investigation. The pH optimum was investigated in the pH 2-10 range, and for a few samples both lower and higher pH values were included to positively identify the optimum. The results are shown in this Table 9.

TABLE 9
pH optimum of beta-glucanases (lichenases):

| | Mw, | | A595/ | pH | pH 10/ |
|---|---|---|---|---|---|
| Organism | kDa | pI | mg | optimum | pHopt |
| Bacillus amyloliquefaciens | 24 | 5.2 | 765 | 6 | 0.01 |
| Bacillus subtilis | 24 | 6.1 | 242 | 6 | 0.11 |
| Bacillus sp-62449 | 40 | 4.4 | 763 | 8 | 0.73 |
| Bacillus akibai | 29 | 5.2 | 5 | 6-9 | 0.9 |
| Bacillus agaradhaerens | 27 | 4.5 | 106 | 9 | 0.68 |
| Bacillus mojavensis | 25 | 7.4 | 313 | 8 | 0.23 |

Based on the above a number of observations were made:

The beta-glucanase from Bacillus amyloliquefaciens and Bacillus subtilis was found to have a pH optimum of 6.0, and relative to this activity only between 1-11% percent activity at pH 10.0. The new bacterial beta-glucanases were found to have pH optimum ranging from pH 6-9, but with a significantly higher relative activity at pH 10 ranging from 23-90% compared to the enzymes from Bacillus subitilis and Bacillus amyloliquefaciens. The GH16 beta-glucanase from B. akibai had a very broad pH optimum.

### Example 9

### Identification, Generation and Screening of Variants

Two conserved methionines (M29, M180) were identified in polypeptides with SEQ ID NO: 23 and SEQ ID NO: 24 by using multiple sequence alignment (MUSCLE) (e.g. FIG. 1) and structural modeling based on known structures of B. subtilis (3O5S) and B. licheniformis (1GBG) beta-glucanases. These partly conserved methionines (M29, M180) stick their side chains and thus the oxidation labile sulphur atoms into the substrate binding clefts in close vicinity to the active side residues. An oxidation of any one or both of these methionines affects the substrate binding and leads to reduced activity and/or stability.

These methionines (M29, M180) in polypeptides with SEQ ID NO: 23 and SEQ ID NO: 24 were used to identify corresponding amino acid residues in mature polypeptides with SEQ ID NO: 25, SEQ ID NO: 26, SEQ ID NO: 27 and SEQ ID NO: 28 (e.g. FIG. 1). Accordingly, as shown in FIG. 1 the following corresponding amino acid residues were identified: M32 and M188 of the polypeptide of SEQ ID NO: 25; F33 and M188 of the polypeptide of SEQ ID NO: 26; F33 and M188 of the polypeptide of SEQ ID NO: 27; and M29 and M180 of the polypeptide of SEQ ID NO: 28. It is therefore suggested to substitute an amino acid residue at one or more positions corresponding to positions selected from a group consisting of: M32 and M188 of the polypeptide of SEQ ID NO: 25; F33 and M188 of the polypeptide of SEQ ID NO: 26; F33 and M188 of the polypeptide of SEQ ID NO: 27 and M29 and M180 of the polypeptide of SEQ ID NO: 28, either alone or in combination, e.g., in small libraries, e.g. M32X+M188X, F33X+M188X, or M29X+M180X, which are then to be screened for stability in the presence of bleach. Such libraries can be made with changes in M32X and M188X, F33X and M188X, M29X and M180X, where X can be any amino acid by NNS doping of the methionine (or phenilalanine) codons such that PCR fragments expanding the two positions can be obtained. Upper and lower integration fragments can be prepared by PCR using primers specific for the parent glucanase gene and overlapping with the M32X+M188X, F33X+M188X and M29X-M180X fragments, and primers for specific sites in the Bacillus genome. The glucanase gene expression cassette can be constructed by triple SOE (splicing by overlap extension) PCR method using primers specific for the upper and lower pectate lyase gene and the derived PCR fragments can be transformed into a suitable B. subtilis host where the expression construct can be integrated into the Bacillus subtilis chromosome by homologous recombination into the pectate lyase (pel) locus. The gene coding for chloramphenicol acetyltransferase can be used as maker (as described in (Diderichsen et al., 1993, Plasmid 30: 312-315). Chloramphenicol resistant clones can be analyzed by DNA sequencing.

Libraries of variants can be screened for beta-glucanase activity and stability using established assays and reference activity and stability (e.g. those of the wild type beta-glucanase from B. amyloliquefaciens) as described below. A preferred assay for measuring beta-glucanase activity of variants is disclosed in example 1 above. A preferred assay for refining substrate specificity of beta-glucanase activity of variants is disclosed in example 6 above.

Alternatively, beta-glucanase activity of variants can be measured in a microtiterplate (MTP) format assay using highly purified low viscosity barlet 1,3-1,4-β-glucan dyed with Remazolbrilliant Blue R dye in form of Tablets (Megazyme, Wicklow, Ireland).

The assay can be performed in 100 mM B&R buffer pH 7.5, prepared according to H. T. S. Britton and R. A. Robinson, J. Chemn. Soc. 1931, 1456-1462. Method steps can be the following:

1. Diluting samples in B&R-buffer to reach an activity in the linear range (i.e. the absorbance to be measured under item 9 should be in the range of 0.1 to 1.0);

2. Preparing substrate: 1 tablet in 5 ml B&R-buffer containing 0.24 mM CaCl2, Brij pH 7.5;

3. Under constant stirring, transferring 130 μl substrate to 96 PCR tubes. Keeping it cool;

4. Adding 20 μl of diluted enzyme. Putting caps on and inverting 2-3 times to mix;

5. Incubating at 40° C. for 10 min in thermocycler;

6. Adding 75 μl of NaOH 1M, sealing the tubes and inverting 2-3 times to mix;

7. Centrifuging for 5 min at 2500 rpm;

8. Transferring 100 μl to a new microtiter plate

9. Measuring absorbance at 590 nm.

Based on the above MTP-plates with variants and beta-glucanase from B. amyloliquefaciens as reference and the buffer as a blind sample, can be incubated in 120 μl Med-F media (e.g. as in Table 10 below) for 3 days, 220 rpm and 37° C. in a shaker with humidity control.

The supernatant can be diluted 100× in buffer (100 mM BR, 0.24 mM CaCl2, pH 7.5) and secondly diluted 1:1 with a detergent/bleach solution. Two identical samples can be prepared.

One sample can be kept at 4° C. until use while the other sample can be heated for 20 minutes at 40° C. in a PCR-machine, to evaluate stability of the variants. Consequently, variants which have both activity and high residual activity can be identified and substitution in e.g. M32X+M188X, F33X+M188X, or M29X+M180X can be confirmed by sequencing.

TABLE 10
Med-F fermentation media can be prepared as follows

| Compound | Amount added for preparing 1 liter | |
|---|---|---|
| Maltodextrin | 12.2 | g |
| Casitone | 6.94 | g |
| Peptone Bacto | 0.56 | g |
| Yeast Extract | 0.56 | g |
| Magnesium sulfate (MgSO₄ × 7H₂O) | 0.56 | g |
| Calcium chlorid (CaCl₂ × 2H₂O) | 0.11 | g |
| Water - to: | 1000 | ml |

Detergent/bleach solution: 0.8 g/100 m1 solution can be made of phosphate-free detergent without bleach, 0.08 g (10%) percarbonat (DC01596) (bleach)+0.032 g (4%) TAED (bleach activator) and diluted up to 100 ml using Milli-Q water. Chemicals can be purchased from Difco or Merck. The detergent/bleach should be prepared fresh for each experiment.

The invention described and claimed herein is not to be limited in scope by the specific aspects herein disclosed, since these aspects are intended as illustrations of several aspects of the invention. Any equivalent aspects are intended to be within the scope of this invention. Indeed, various modifications of the invention in addition to those shown and described herein will become apparent to those skilled in the art from the foregoing description. Such modifications are also intended to fall within the scope of the appended claims. In the case of conflict, the present disclosure including definitions will control.

1. A variant of a parent beta-glucanase, the variant comprising a substitution at one or more positions corresponding to positions 33 and 188 of the mature polypeptide of SEQ ID NO: 26 using the numbering of SEQ ID NO: 26, wherein the variant has beta-glucanase activity and wherein the variant has at least 80%, but less than 100% sequence identity to the mature polypeptide of any of: SEQ ID NO: 26, SEQ ID NO: 27, SEQ ID NO: 25, and SEQ ID NO: 28.

2. The variant of claim 1, wherein said parent beta-glucanase is selected from the group consisting of:
i) a polypeptide having at least 85% sequence identity to the mature polypeptide selected from the group consisting of: SEQ ID NO: 26, SEQ ID NO: 27, SEQ ID NO: 25 and SEQ ID NO: 28; and
ii) a fragment of the polypeptide selected from the group consisting of: SEQ ID NO: 26, SEQ ID NO: 27, SEQ ID NO: 25 and SEQ ID NO: 28, wherein said fragment has beta-glucanase activity.

3. The variant of claim 1, wherein the parent beta-glucanase comprises or consists of the polypeptide selected from the group consisting of: SEQ ID NO: 26, SEQ ID NO: 27, SEQ ID NO: 25 and SEQ ID NO: 28.

4. The variant of claim 1, which comprises a substitution at a position corresponding to position 33, wherein the substituent amino acid is any of: Ala, Arg, Asn, Asp, Cys, Gln, Glu, Gly, His, Ile, Leu, Lys, Pro, Ser, Thr, Trp, Tyr or Val.

5. The variant of claim 1, which comprises a substitution at a position corresponding to position 188, wherein the substituent amino acid is any of: Ala, Arg, Asn, Asp, Cys, Gln, Glu, Gly, His, Ile, Leu, Lys, Phe, Pro, Ser, Thr, Trp, Tyr or Val.

6. The variant of claim 1, which comprises or consists of a substitution selected from the group consisting of: F33V+M188L; F33A+M188F; F33Y; F33V+M188H; F33G+M188L; F33N; F33G+M188R; F33S+M188Y; F33G+M188H; F33E+M188L; M188H; F33W+M188S; F33N+M188F; F33S+M188A; F33C+M188L; F33V+M188T; F33Q+M188R; F33L+M188T; F33G+M188C; F33N+M188Q; and F33L+M188A.

7. The variant claim 1, which has an improved property relative to the parent, wherein the improved property is increased oxidation stability.

8. The variant of claim 1, wherein said beta-glucanase activity is licheninase EC 3.2.1.73 activity.

9. A composition comprising the variant of claim 1.

10. The composition of claim 9, further comprising: i) one or more detergent components; or ii) one or more additional enzymes.

11. The composition of claim 9, wherein said composition is a cleaning or detergent composition.

12. The composition of claim 9, further comprising one or more amylases.

13. The composition of claim 9, wherein said composition is a liquid composition or a solid composition.

14. A method for obtaining a beta-glucanase variant, comprising introducing into a parent beta-glucanase a substitution at one or more positions corresponding to positions 33 and 188 of the mature polypeptide of SEQ ID NO: 26 using the numbering of SEQ ID NO: 26, wherein the variant has beta-glucanase activity; and recovering the variant.

15. The method of claim 14, wherein the variant has at least 80%, but less than 100% sequence identity to the mature polypeptide of any of: SEQ ID NO: 26, SEQ ID NO: 27, SEQ ID NO: 25, and SEQ ID NO: 28.

16. (canceled)

17. A method for cleaning, comprising contacting an object with the composition of claim 9.
